(19) United States
(12) Patent Application Publication
(10) Pub. No.: US 2019/0169602 A1
(43) Pub. Date: Jun. 6, 2019

(54) COMPOSITIONS AND METHODS FOR SEQUENCING NUCLEIC ACIDS

(71) Applicant: seqWell, Inc., Beverly, MA (US)

(72) Inventors: Joseph C. MELLOR, Melrose, MA (US); Jack T. LEONARD, South Hamilton, MA (US)

(21) Appl. No.: 16/069,603

(22) PCT Filed: Jan. 12, 2017
(86) PCT No.: PCT/US2017/013191

§ 371 Date: Jul. 12, 2018

Related U.S. Application Data
(60) Provisional application No. 62/411,399, filed on Oct. 21, 2016.
(60) Provisional application No. 62/277,867, filed on Jan. 12, 2016.

(51) Int. Cl. C12N 15/10; C12N 15/11; C12Q 1/6869

(52) U.S. Cl. CPC C12N 15/1065; C12Q 1/6869; C12N 15/11

(57) ABSTRACT

The invention provides compositions, including tethered synaptic complexes (TSCs), artificial nucleic acids, molecular constructs that include artificial nucleic acids bound to transposases, and kits; as well as methods of using the same, for example, for preparation of nucleic acid libraries and sequencing.

### FIELD OF THE INVENTION

The present invention relates generally to nucleic acid (e.g., DNA) sequencing and, more specifically, to artificial nucleic acids, compositions that include artificial nucleic acids and transposases, and methods of use thereof, e.g., for library preparation and sequencing.

### BACKGROUND

Nucleic acid (e.g., DNA) sequencing has become an indispensable part of modern biology, and has wide uses, for example, identification and classification of species (e.g., pathogens), identification of genetic abnormalities such as disease-associated mutations, measuring RNA transcripts present in a cell, among many others. Current approaches include massively parallel or “next-generation” sequencing (NGS), which allow for parallel processing of many nucleic acids in a single sequencing run. NGS has revolutionized genomics and molecular biology by greatly increasing the speed of sequencing while reducing costs. In general, NGS approaches involve preparing a library of template nucleic acids from a target nucleic acid to be sequenced, obtaining sequence data from the library, and assembling the sequence data to infer the sequence of the target nucleic acid. Most NGS approaches utilize sequencing libraries having small fragments (typically on the order of hundreds of base pairs), in part due to technical limitations of the approaches. The resulting short reads are assembled computationally, often by alignment to a reference sequence, to infer the sequence of the target nucleic acid.

One of the limitations of current library preparation approaches for NGS is that each of the fragments in the library typically represents only a very small piece of a much larger original source target nucleic acid. For example, the fragments in the library may be only a few hundred nucleotides long whereas the source target nucleic acid(s) may have been a chromosome or an entire genome. This makes it difficult to use current library preparation methods to sequence DNA, and particularly whole genomes, because the contiguity of bases over longer distances (e.g., thousands or millions of bases) can only be inferred computationally by attempting to overlap smaller fragments (in a computational process called de novo sequence assembly). The inherent “short range” limitation of conventional NGS library preparation methods limits the use of current DNA sequencing methods to those that can be carried out using relatively homogeneous, high purity samples. Additionally, the small size of library fragments makes it highly unlikely that sequenced library fragments originate from the same target nucleic acid molecule.

Therefore, there remains a need for compositions and methods useful for library preparation and sequencing that can obtain long distance linkage and sequence information, as well as for preparing libraries having a high proportion of fragments originating from the same target nucleic acid molecule.

### SUMMARY OF THE INVENTION

The present invention provides compositions and methods of use thereof, e.g., for nucleic acid library preparation and sequencing.

In one aspect, the invention features a tethered synaptic complex (TSC) including: a first artificial nucleic acid including a first end including a first transposase binding site (TBS), a second end including a second TBS, and a linking segment disposed between the first TBS and the second TBS; a second artificial nucleic acid including a first end including a first TBS; a third artificial nucleic acid including a first end including a first TBS; a first synaptic complex including a first pair of oligomerized transposases, the first pair including a first transposase and a second transposase, wherein the first transposase is bound to the first TBS of the first artificial nucleic acid, and the second transposase is bound to the first TBS of the second artificial nucleic acid; and a second synaptic complex including a second pair of oligomerized transposases, the second pair including a third transposase and a fourth transposase, wherein the third transposase is bound to the second TBS of the first artificial nucleic acid, and the fourth transposase is bound to the first TBS of the third artificial nucleic acid.

In some embodiments, the linking segment includes a nucleic acid. In some embodiments, the nucleic acid is at least partially single-stranded. In other embodiments, the nucleic acid is double-stranded. In some embodiments, the linking segment includes terminal nucleotides that form phosphodiester bonds with the first TBS and the second TBS.

In some embodiments, the linking segment includes an affinity binding pair or a covalent bond resulting from a conjugation reaction that does not form a phosphodiester bond. In some embodiments, the affinity binding pair includes biotin-streptavidin, biotin-avidin, ligand-receptor, antigen-antibody or antigen binding fragment, hapten-anti-hapten, or immunoglobulin (Ig) binding protein-lg. In some embodiments, the affinity binding pair includes biotin-streptavidin or biotin-avidin. In some embodiments, the streptavidin or avidin binds only one or two biotin molecules. In some embodiments, the affinity binding pair includes a first affinity component that binds to two second affinity components, where one second affinity component is linked to the first end of the first artificial nucleic acid, and the other second affinity component is linked to the second end of the first artificial nucleic acid, and wherein the two second affinity components do not interfere with binding of transposases to the first and second TBSs of the first artificial nucleic acid. In some embodiments, the affinity binding pair includes a first affinity component that binds a second affinity component, where the first affinity component is linked to the first end of the first artificial nucleic acid, and the second affinity component is linked to the second end of the first artificial nucleic acid, wherein the first affinity component and the second affinity component do not interfere with binding of transposases to the first and second TBSs of the first artificial nucleic acid. In some embodiments, the conjugation reaction is selected from the group consisting of a cycloaddition, amide or thioamide bond formation, a pericyclic reaction, a Diels-Alder reaction, sulfonamide bond formation, alcohol or phenol alkylation, a condensation reaction, disulfide bond formation, and a nucleophilic substitution. In some embodiments, the cycloaddition is an azide-alkyne Huisgen cycloaddition, e.g., a copper(I)-catalyzed azide-alkyne cycloaddition (CuAAC) or a strain-promoted azide-alkyne cycloaddition (SPAAC).

In some embodiments, the linking segment further includes one or more additional elements selected from the group consisting of an identifiable sequence tag (IST), a primer binding site, a cleavage site (e.g., a restriction endonuclease recognition site or a nickase site), and a chemical modification. In some embodiments, the one or more additional elements is an IST, e.g., a random IST, a semi-random IST, or a non-random IST.

In certain embodiments, the first artificial nucleic acid is about 50 to about 500 base pairs (bp) long, e.g., about 100 to about 250 bp long, about 150 to about 200 bp long, or about 175 bp long. In some embodiments, the transposases of the first pair are a different type than the transposases of the second pair. In some embodiments, the transposases of the first pair and/or the second pair are Tn3, Tn5, Tn9, Tn10, gamma-delta, Mu, piggyBac, Minos, Tc1, or Sleeping Beauty transposases or biologically active variants thereof. In some embodiments, the transposases of the first pair and/or the second pair are Tn3, Tn5, Tn9, Tn10, gamma-delta, or Mu transposases or biologically active variants thereof. In some embodiments, the transposases of the first pair and/or the second pair are Tn5 or Mu transposases or biologically active variants thereof. In some embodiments, the transposases of the first pair are Tn5 transposases, and the transposases of the second pair are Mu transposases.

In certain embodiments, at least one transposase of the first pair and/or the second pair is operably linked to a targeting moiety, e.g., a polypeptide including a DNA-binding domain (DBD) or an RNA-guided endonuclease. In some embodiments, the DBD is a zinc finger motif or a transcription activator-like (TAL) effector. In some embodiments, the RNA-guided endonuclease is Cas9, Cpf1, C2c2, or a biologically active variant thereof, e.g., a nuclease-deficient variant.

In some embodiments, the second artificial nucleic acid or the third artificial nucleic acid further includes a second end that is a ligatable end, such as a sticky end.

In some embodiments, the second artificial nucleic acid or the third artificial nucleic acid further includes a component of a second affinity binding pair or a conjugating moiety, e.g., a conjugating moiety selected from the group consisting of a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a sulfhydryl, a thiol, and a thioisocyanate group.

In some embodiments, the second and/or third artificial nucleic acid further includes a second end including a second TBS, and a linking segment disposed between the first TBS and the second TBS of the second and/or third artificial nucleic acid. In some embodiments, the linking segment of the second and/or third artificial nucleic acid includes a second affinity binding pair or a covalent bond resulting from a conjugation reaction, wherein the covalent bond is not a phosphodiester bond.

In certain embodiments, the TSC further includes one or more additional synaptic complexes including a pair of oligomerized transposases, and/or one or more additional artificial nucleic acids including a TBS at each end and an intervening linking segment. In this embodiment, the first synaptic complex, the second synaptic complex, and the one or more additional synaptic complexes are tethered to at least one other synaptic complex of the TSC by binding to TBSs at either end of the same artificial nucleic acid. In some embodiments, the TSC includes between one and ten thousand additional synaptic complexes. In some embodiments, each artificial nucleic acid of the TSC includes an IST. In some embodiments, each IST is identical. In other embodiments, each IST is not identical.

In some embodiments, the linking segment of the first artificial nucleic acid is soluble in an aqueous solution.

In another aspect, the invention provides an artificial nucleic acid including a first end including a first TBS, a second end including a second TBS, and a linking segment disposed between the first TBS and the second TBS, wherein upon binding of a first transposase to the first TBS and a second transposase to the second TBS, the first transposase does not oligomerize with the second transposase. In some embodiments, the linking segment includes a nucleic acid. In some embodiments, the nucleic acid is at least partially single-stranded. In other embodiments, the nucleic acid is double-stranded. In some embodiments, the linking segment includes terminal nucleotides that form phosphodiester bonds with the first TBS and the second TBS. In some embodiments, the linking segment includes an affinity binding pair or a covalent bond resulting from a conjugation reaction that does not form a phosphodiester bond. In some embodiments, the affinity binding pair includes biotin-streptavidin, biotin-avidin, ligand-receptor, antigen-antibody or antigen binding fragment, hapten-anti-hapten, or immunoglobulin (Ig) binding protein-lg. In some embodiments, the affinity binding pair includes biotin-streptavidin or biotin-avidin. In some embodiments, the streptavidin or avidin binds only one or two biotin molecules. In some embodiments, the affinity binding pair includes a first affinity component that binds to two second affinity components, where one second affinity component is linked to the first end of the first artificial nucleic acid, and the other second affinity component is linked to the second end of the first artificial nucleic acid, and wherein the two second affinity reagents do not interfere with binding of transposases to the first and second TBSs of the first artificial nucleic acid. In some embodiments, the affinity binding pair includes a first affinity component that binds a second affinity component, where the first affinity component is linked to the first end of the first artificial nucleic acid, and the second affinity component is linked to the second end of the first artificial nucleic acid. In some embodiments, the conjugation reaction is selected from the group consisting of a cycloaddition, amide or thioamide bond formation, a pericyclic reaction, a Diels-Alder reaction, sulfonamide bond formation, alcohol or phenol alkylation, a condensation reaction, disulfide bond formation, and a nucleophilic substitution. In some embodiments, the cycloaddition is an azide-alkyne Huisgen cycloaddition, e.g., a copper(I)-catalyzed azide-alkyne cycloaddition (CuAAC) or a strain-promoted azide-alkyne cycloaddition (SPAAC).

In some embodiments, the linking segment further includes one or more additional elements selected from the group consisting of an IST, a primer binding site, a cleavage site, and a chemical modification. In some embodiments, the one or more additional elements includes an IST, such as a random IST, a semi-random IST, or a non-random IST. In some embodiments, the cleavage site is a restriction endonuclease recognition site or a nickase site.

In some embodiments, the nucleic acid is about 50 bp to about 500 bp in length, about 100 to about 250 bp long, about 150 to about 200 bp long, or about 175 bp long.

In some embodiments, the linking segment prevents the first transposase and the second transposase from oligomerizing when bound to the first TBS and the second TBS. In some embodiments, the first transposase and the second transposase do not oligomerize with each other.

In some embodiments, the first TBS or the second TBS is double-stranded.

In some embodiments, more than one transposase binds to the first TBS or the second TBS.

In yet another aspect, the invention features a molecular construct including a first transposase, a second transposase, and any one of the preceding artificial nucleic acids, wherein the first transposase is bound to the first TBS and the second transposase is bound to the second TBS. In some embodiments, the linking segment prevents the first transposase and the second transposase from oligomerizing with each other. In some embodiments, the first transposase and the second transposase do not oligomerize with each other. In some embodiments, the first transposase or the second transposase is a Tn3, Tn5, Tn9, Tn10, gamma-delta, Mu, piggyBac, Minos, Tc1, or Sleeping Beauty transposase or a biologically active variant thereof, e.g., a Tn3, Tn5, Tn9, Tn10, gamma-delta, or Mu transposase or a biologically active variant thereof. In some embodiments, the first transposase or the second transposase is a Tn5 transposase, a Mu transposase, or a biologically active variant thereof. In some embodiments, the first transposase is a Tn5 transposase, and the second transposase is a Mu transposase. In some embodiments, more than one transposase binds to the first TBS or the second TBS. In some embodiments, the first transposase or the second transposase is operably linked to a targeting moiety. In some embodiments, the targeting moiety is a polypeptide including a DNA-binding domain (DBD) or an RNA-guided endonuclease. In some embodiments, the DBD is a zinc finger motif or a transcription activator-like (TAL) effector. In some embodiments, the RNA-guided endonuclease is Cas9, Cpf1, C2c2, or a biologically active variant thereof, e.g., a nuclease-deficient variant.

In a further aspect, the invention provides a TSC including at least three of the preceding molecular constructs, wherein the constructs are concatenated by oligomerization of a transposase in each construct with a transposase in another construct, and wherein at least two synaptic complexes are present in the TSC. In certain embodiments, the TSC includes between two and ten thousand synaptic complexes. In some embodiments, each artificial nucleic acid in the TSC includes an IST. In some embodiments, each IST is identical. In other embodiments each IST is not identical.

In another aspect, the invention provides a ligatable synaptic complex including: a first transposase; a second transposase; a first artificial nucleic acid including a first end including a TBS and a second end that is a sticky end; and a second artificial nucleic acid including a first end including a TBS and a second end that is a sticky end, wherein the first transposase is bound to the TBS of the first artificial nucleic acid, the second transposase is bound to the TBS of the second artificial nucleic acid, and the first transposase and the second transposase are oligomerized.

In a still further aspect, the invention provides a method of preparing a TSC by providing at least a first synaptic complex and a second synaptic complex, wherein the first synaptic complex includes a first pair of oligomerized transposases, wherein one member of the pair is bound to a first nucleic acid including a TBS and a sticky end; and the second synaptic complex includes a second pair of oligomerized transposases, wherein one member of the pair is bound to a second nucleic acid including a TBS and a sticky end; and ligating the first synaptic complex to the second synaptic complex by ligating the sticky ends of the first nucleic acid and the second nucleic acid in a ligation reaction. In some embodiments, the first nucleic acid and the second nucleic acid have the same nucleic acid sequence. In other embodiments, the first nucleic acid and the second nucleic acid have different nucleic acid sequences. In certain embodiments, the providing further includes providing a linking segment nucleic acid including a first sticky end and a second sticky end, wherein the first sticky end is compatible with the sticky end of the first nucleic acid, and the second sticky end is compatible with the sticky end of the second nucleic acid. In some embodiments, the ligating further includes ligating the linking segment nucleic acid to the first nucleic acid and the second nucleic acid. In some embodiments, the linking segment includes one or more additional elements selected from the group consisting of IST, a primer binding site, a cleavage site, or a chemical modification. In some embodiments, the chemical modification is a biotinylation.

In a still further aspect, the invention provides a synaptic complex including a first transposase; a second transposase; a first artificial nucleic acid including a first end including a TBS and being linked to a first conjugating moiety other than a 3′ hydroxyl; and a second artificial nucleic acid including a first end including a TBS and being linked to a second conjugating moiety other than a 3′ hydroxyl, wherein the first transposase is bound to the TBS of the first artificial nucleic acid, the second transposase is bound to the TBS of the second artificial nucleic acid, and the first transposase and the second transposase are oligomerized. In some embodiments, the first and second conjugating moieties are the same or different. In some embodiments, the first and second conjugating moieties are independently selected from the group consisting of a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a sulfhydryl, a thiol, and a thioisocyanate group. In some embodiments, the first or second artificial nucleic acid further includes an IST.

In a further aspect, the invention provides a method of preparing a TSC by providing at least a first synaptic complex and a second synaptic complex, wherein the first synaptic complex includes a first pair of oligomerized transposases, wherein one member of the pair is bound to a first nucleic acid including a TBS and being linked to a first component of an affinity binding pair or a first conjugating moiety; and the second synaptic complex includes a second pair of oligomerized transposases, wherein one member of the pair is bound to a second nucleic acid including a TBS and being linked to a second component of the affinity binding pair or a second conjugating moiety; and linking the first synaptic complex to the second synaptic complex, wherein linking of the first conjugating moiety to the second conjugating moiety does not result in a phosphodiester bond. In some embodiments, the linking includes combining the first synaptic complex and the second synaptic complex under conditions suitable for binding of the first component and the second component of the affinity binding pair. In other embodiments, the linking includes conjugating the first conjugating moiety to the second conjugating moiety in a conjugation reaction that does not produce a phosphodiester bond.

In another aspect, the invention provides a TSC prepared by any one of the methods described herein.

In another aspect, the invention features a method of preparing a target nucleic acid for sequencing by combining any one of the TSCs described herein with a target nucleic acid under conditions and for a time sufficient for the TSC to carry out a transposition event. In certain embodiments, the method further includes fragmenting the target nucleic acid and adding a polynucleotide to the resulting ends of the nucleic acid fragments. In some embodiments, the fragmenting may include random shearing and adapter ligation or tagmentation.

In yet another aspect, the invention features a method of sequencing a target nucleic acid by combining any one of the TSCs described herein with a target nucleic acid under conditions and for a time sufficient for the TSC to carry out a transposition event; fragmenting the target nucleic acid and adding a polynucleotide to the resulting ends of the nucleic acid fragments; selecting DNA fragments including a nucleic acid sequence resulting from the transposition event; amplifying the selected fragments; and sequencing the amplified fragments. In some embodiments, the fragmenting may include random shearing and adapter ligation or tagmentation. In some embodiments, the selecting includes selecting nucleic acid fragments including an IST. In some embodiments, the amplifying includes polymerase chain reaction (PCR), multiple displacement amplification (MDA), ligase chain reaction (LCR), loop mediated isothermal amplification (LAMP), rolling circle amplification (RCA), or strand displacement amplification (SDA). In some embodiments, the sequencing includes sequencing by synthesis, sequencing by ligation, or nanopore sequencing. In some embodiments, the sequencing by synthesis includes Illumina™ dye sequencing, single-molecule real-time (SMRT™) sequencing, or pyrosequencing. In some embodiments, the sequencing by ligation includes polony-based sequencing or SOLiD™ sequencing. In certain embodiments, the method further includes analyzing the sequenced fragments to identify fragments of the target nucleic acid that can be linked by the presence of a nucleic acid sequence resulting from the transposition event.

In some embodiments of any of the preceding methods, the target nucleic acid includes genomic DNA or cDNAs from a single cell. In other embodiments, the target nucleic acid includes nucleic acids from a plurality of haplotypes. In some embodiments, the sequence of the amplified fragments is used to perform de novo sequence assembly.

In another aspect, the invention provides a kit including any one of the preceding TSCs.

In yet another aspect, the invention provides a kit including any one of the preceding nucleic acids and a purified transposase that binds to the first TBS or the second TBS.

In another aspect, the invention provides a kit including any one of the preceding molecular constructs.

Any of the kits described herein can further include one or more additional reagents selected from the group consisting of a cofactor, a buffered solution, and a reference nucleic acid. In some embodiments, the cofactor is a divalent metal cation (e.g., a magnesium cation).

Any of the kits described herein can further include reagent for nucleic acid sequencing. In some embodiments, the reagent is selected from the group consisting of an oligonucleotide primer, a substrate, an enzyme, and a mixture of nucleotides.

### Definitions

The term “about” is used herein to indicate that a value includes an inherent variation of error for the device or the method being employed to determine the value or to indicate plus-or-minus 10% of the stated value, whichever is greater.

The term “affinity binding pair” refers to a pair of moieties that bind and form a complex. In general, the affinity binding pairs used in the invention interact non-covalently. Exemplary affinity binding pairs include, without limitation, biotin-biotin binding protein (e.g., biotin-streptavidin and biotin-avidin), ligand-receptor, antigen-antibody or antigen binding fragment, hapten-anti-hapten, and immunoglobulin (Ig) binding protein-lg. The members of an affinity binding pair may have any suitable binding affinity. For example, the members of an affinity binding pair may bind with an equilibrium binding constant (K_{D}) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or lower.

“Amino acid sequence,” as used herein, refers to a peptide, polypeptide, or protein sequence, and fragments or portions thereof, and to naturally occurring or synthetic molecules. The terms “protein” and “polypeptide” are used interchangeably herein.

The term “biologically active variant” refers to a moiety that is similar to, but not identical to, a reference moiety (e.g., a “parent” molecule or template) and that exhibits sufficient activity to be useful in one or more of the compositions or methods described herein (e.g., in place of the reference moiety). In some instances, the reference moiety is naturally occurring, and the biologically active variant thereof is not. For example, where the reference moiety is a naturally occurring nucleic acid sequence, a biologically active variant thereof can include a limited number of non-naturally occurring nucleotides; can have a nucleic acid sequence that differs from its naturally occurring counterpart (e.g., by one or more insertions, deletions, and/or substitutions); or can otherwise vary from its naturally occurring counterpart.

For example, the nucleic acids described herein (and the molecular constructs and tethered synaptic complexes which contain them) can include a transposase binding site (TBS) that differs from a naturally occurring TBS but nevertheless retains the ability to bind a transposase and to function in the present compositions and methods. Where the reference moiety is a naturally occurring protein, a biologically active variant thereof can include a limited number of non-naturally occurring amino acids; can have a peptide sequence that differs from its naturally occurring counterpart; or can otherwise vary from its naturally occurring counterpart (e.g., by virtue of being modified post-translationally (e.g., its glycosylation pattern may differ)). The reference moiety may also be non-naturally occurring.

A “conjugation reaction” is a reaction that results in the formation of a covalent bond. For the purposes of the present disclosure, a conjugation reaction excludes formation of a phosphodiester bond.

Non-limiting examples of conjugation reactions include cycloaddition (e.g., an azide-alkyne Huisgen cycloaddition (e.g., a copper(I)-catalyzed azide-alkyne cycloaddition (CuAAC) or a strain-promoted azide-alkyne cycloaddition (SPAAC))), amide or thioamide bond formation, a pericyclic reaction, a Diels-Alder reaction, sulfonamide bond formation, alcohol or phenol alkylation, a condensation reaction, disulfide bond formation, and a nucleophilic substitution.

A “distal site” is a location on a target DNA that is situated between about 100 base pairs (bp) and about 20 million bp from a reference point. For example, a distal site may be about 100 bp, about 200 bp, about 500 bp, about 1000 bp, about 5000 bp, about 10,000 bp, about 20,000 bp, about 50,000 bp, about 100,000 bp, about 250,000 bp, about 500,000 bp, about 750,000 bp, about 1 million bp, about 5 million bp, about 10 million bp, about 15 million bp, or about 20 million bp from a reference point. Two sites (e.g., “A” and “B”) may be referred to as distal sites when A is situated between about 100 bp and 20 million bp away from B.

An “identifiable sequence tag” (IST) refers to any nucleic acid sequence that can be identified and used as a marker that a transposable nucleic acid has transposed into a target nucleic acid. The IST may be random, semi-random, or non-random. In some embodiments, an IST may be a nucleic acid barcode.

An IST can include, for example, about 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, or more consecutive nucleotides. A transposable nucleic acid may include, for example, 1, 2, 3, 4, 5, 6, 7, 8, 9, 10, or more ISTs.

As used herein, the term “fusion protein” refers to a composition containing all or a portion of the amino acid sequences of two or more proteins. For example, a fusion protein may include a transposase and a polypeptide targeting moiety. A fusion protein may include one or more linkers between the amino acid sequences of the proteins. The term “portion” includes any region of a polypeptide, such as a fragment (e.g., a cleavage product or a recombinantly-produced fragment) or an element or domain (e.g., a region of a polypeptide having an activity, for example, nucleic acid (e.g., DNA) binding), that contains fewer amino acids than the full-length or reference polypeptide (e.g., about 5%, 10%, 15%, 20%, 30%, 40%, 50%, 60%, 70%, 80%, 90%, 95%, or 99% fewer amino acids).

The term “ligatable,” as used herein, means that a moiety is capable of being ligated to another moiety in a ligation reaction, resulting in formation of a phosphodiester bond. A ligation reaction typically involves ligation by a DNA ligase (e.g., T4 DNA ligase) under suitable conditions to form a phosphodiester bond. For example, a first artificial nucleic acid may include a ligatable end that can be ligated to a ligatable end of a second artificial nucleic acid. A ligatable end of a nucleic acid may be a blunt end or a sticky end, for example. In some instances, a ligatable synaptic complex may include at least one transposase that is bound to a ligatable nucleic acid.

The terms “linking segment” and “linker,” as used interchangeably herein, refers to an element that is disposed between two sequences (e.g., nucleic acid or polypeptide sequences) and which links the two sequences. The linkage can be covalent or non-covalent. A linking segment can include, for example, a nucleotide, a nucleic acid, a non-nucleotide chemical moiety (e.g., a polymer, e.g., a polyether such as polyethylene glycol (PEG)), an amino acid, peptide, or polypeptide. A nucleic acid linking segment can include, for example, about 1, 2, 3, 4, 5, 10, 15, 20, 25, 30, 35, 40, 45, 50, 55, 60, 65, 70, 75, 80, 85, 90, 100, 120, 140, 160, 180, 200, 225, 250, 275, 300, 400, 500, 1000, 2000, 5000, or more nucleotides. A polypeptide linking segment can include, for example, about 1, 2, 3, 4, 5, 10, 15, 20, 25, 30, 35, 40, 45, 50, 55, 60, 65, 70, 75, 80, 85, 90, 100, 120, 140, 160, 180, 200, 225, 250, 275, 300, 400, 500, 1000, or more amino acids. A linking segment may include one or more affinity binding pairs and/or covalent bonds resulting from conjugation reactions. In some instances, a linking segment is soluble in aqueous solution. As used herein, the term “linking segment” excludes solid materials having a mass of greater than about 15 femtograms (fg). For example, a linking segment can have a mass of about 15 fg or less, about 14 fg or less, about 13 fg or less, about 12 fg or less, about 11 fg or less, about 10 fg or less, about 9 fg or less, about 8 fg or less, about 7 fg or less, about 6 fg or less, about 5 fg or less, about 4 fg or less, about 3 fg or less, about 2 fg or less, about 1 fg or less, about 1×10⁻¹⁶ g or less, about 1×10⁻¹⁷ grams or less, about 1×10⁻¹⁸ grams or less, about 1×10⁻¹⁹ grams or less, or about 1×10⁻²⁰ grams or less.

In particular instances, a linking segment specifically excludes a solid support (e.g., a bead).

The terms “nucleic acid” and “polynucleotide,” as used interchangeably herein, refer to at least two linked nucleotide monomers. The term encompasses, for example, deoxyribonucleic acid (DNA), ribonucleic acid (RNA), hybrids thereof, and mixtures thereof. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5-hydroxymethylcytosine).

An “artificial nucleic acid” refers to a non-naturally occurring nucleic acid. Such artificial nucleic acids differ in some respect from nucleic acids that occur in nature without human intervention, whether by sequence, chemical composition, and/or functional properties.

The terms “operable linkage” and “operably linked,” as used herein, refer to a physical or functional juxtaposition of the components so described as to permit them to function in their intended manner. For example, a targeting moiety may be operably linked with a transposase (e.g., by being fusion partners in a fusion protein or by being otherwise covalently or non-covalently conjugated) in order to promote transposition at a specific sequence in a target nucleic acid (e.g., DNA).

By “synaptic complex” is meant a structure that includes a pair of oligomerized transposases (e.g., dimerized transposases or a tetramer (e.g., dimer of dimers) of transposases) in which each transposase of the pair is bound to a TBS. In nature, a nucleic acid that includes two TBSs may form a synaptic complex by oligomerization of the transposases that bind to each TBS, which results in looping of the nucleic acid. In the context of the present invention, a synaptic complex includes a pair of oligomerized transposases in which each transposase is bound to a TBS present on a different nucleic acid molecule. Accordingly, a synaptic complex constitutes a part of a larger molecular complex as described herein. For example, two synaptic complexes can be tethered by a nucleic acid having a TBS at each terminus to generate a TSC as described below such that, when combined with a target nucleic acid (e.g., DNA), the TSC exhibits transposase activity, cleaving the target nucleic acid, and ligating the tethering nucleic acid (which may include, for example, identifiable sequence tags) to distal sites within the target nucleic acid (e.g., DNA).

A “targeting moiety” refers to any compound (e.g., nucleic acid or polypeptide) that can promote preferential or specific binding to a nucleic acid sequence. For example, a targeting moiety may be a polypeptide that includes a DNA binding domain (DBD), for example, a zinc finger motif or a transcription activator-like (TAL) effector protein; an RNA-guided endonuclease (e.g., Cas9, Cpf1, and C2c2), DNA-guided endonuclease (e.g., Argonaute), or biologically active variants thereof, including nuclease-deficient or nuclease-null variants; or an oligonucleotide (e.g., RNA or DNA) that hybridizes to a nucleic acid sequence.

A “target nucleic acid” refers to any nucleic acid (e.g., DNA) of interest that is selected for modification or analysis (e.g., sequence analysis) using a composition of the invention (e.g., a TSC) as described herein. The present methods can be carried out using target nucleic acids (e.g., DNAs) pooled from more than one source. It is to be understood that the target nucleic acid may be DNA or RNA, for example. In some instances, RNA may be converted to cDNA prior to being treated with a composition of the invention (e.g., a TSC).

A “tethered synaptic complex” (TSC) is a molecular complex that includes a plurality of synaptic complexes (i.e., at least two synaptic complexes) that are tethered “end-to-end” by artificial nucleic acids that include a TBS at each terminus and an intervening linking segment. In some embodiments, the tethering nucleic acids also include a subsequence that includes an identifiable sequence tag. These tags can be used to identify or differentiate one subunit of a TSC from another or, similarly, to identify or differentiate one TSC from another. Further, in certain embodiments, because the identifiable sequence tag in a subunit of the TSC is incorporated into a first site on the target nucleic acid (e.g., DNA), while a complementary identifiable sequence tag is incorporated into a second site on the target nucleic acid (the first and second sites being distal from one another), one can conclude, by virtue of the presence of complementary sequence tags attached to the same TSC, that two sequenced fragments originated from distal sites on the same target nucleic acid molecule. The subsequence can also include a sequence to which a defined oligonucleotide can hybridize in order to serve as, for example, a primer binding site for amplification or sequencing.

“Transferred” or “transposed” nucleic acid is any nucleic acid that is ligated to a target nucleic acid (e.g., DNA) in a transposition event (e.g., in the context of a sequencing method described herein).

A “transposable nucleic acid” is any nucleic acid that can participate in the formation of a functionally active TSC and attach to a target nucleic acid (e.g., DNA) by virtue of including a transposase binding site (TBS) at one or both termini.

The term “transposase” refers to a moiety that binds to a transposase binding site (TBS) and that can catalyze movement of the TBS as well as associated transposable nucleic acid sequence to a different nucleic acid (e.g., DNA) molecule. In nature, transposases bind to TBSs at the ends of a transposon (also known as a transposable element) prior to catalyzing movement the transposon to a different location of the host genome. Transposases typically effect transposition of nucleic acid (e.g., DNA) sequences using a cut and paste mechanism or a replicative transposition mechanism.

Transposases typically catalyze nucleic acid transposition as oligomers. For example, Tn5 transposases catalyze transposition as a dimer, with a monomer binding each TBS. Other transposases, such as Mu (also referred to as MuA), catalyze transposition as a tetramer (dimer of dimers), with a dimer binding each TBS. The term “transposase,” as used herein, refers to the minimal unit that binds to a TBS, and may include, for example, one transposase protein (e.g., a monomer) or more than one transposase protein (e.g., a dimer). Transposases are members of the RnaseH superfamily of proteins, which is characterized by an active site that includes DDE residues that chelate two Mg⁺⁺ ions, which are critical for catalysis, and the overall architecture and active site DDE are considered to be nearly identical to that of retroviral integrases, RuvC, and RnaseH (see, e.g., Reznikoff, Mol. Microbiol. 47(5):1199-1206, 2003).

Given that transposases and retroviral integrases share common active site architecture (including the DDE active site) as well as catalytic mechanisms (e.g., transposon-donor backbone DNA nicking and strand transfer), it is expressly contemplated that retroviral integrases (e.g., human immunodeficiency virus (HIV)-1, HIV-2, simian immunodeficiency virus (SIV), and Rous sarcoma virus integrases) and other related integrases (e.g., integrases of retrotransposons, for example, yeast Ty integrases (e.g., Ty1, Ty2, Ty3, Ty4, and Ty5 integrase)) may also be used in the context of the invention as falling within the scope of “transposase.”

A “transposase binding site” (TBS) is a nucleic acid (e.g., DNA) sequence that can be selectively bound by a transposase. In particular embodiments, the sequence is a DNA sequence. In some instances, under at least a condition specified herein and/or in the context of a sequencing method of the invention, transposase binding sites attached to the target nucleic acid (e.g., DNA) by transposase activity may remain selectively bound by transposases within the TSC.

A “transposition event” is a reaction in which a synaptic complex cleaves a target nucleic acid (e.g., DNA) and ligates a transposable nucleic acid (e.g., all or a part of the transposable nucleic acid, which may include an identifiable sequence tag) to a cleaved target nucleic acid. In particular embodiments, the target nucleic acid is DNA.

### BRIEF DESCRIPTION OF THE DRAWINGS

FIG. 1 illustrates a tethered synaptic complex (TSC).

FIGS. 2A and 2B illustrate exemplary methods by which TSCs can be prepared.

FIG. 3 illustrates additional exemplary methods by which TSCs can be prepared.

FIGS. 4A and 4B illustrate further exemplary methods by which TSCs can be prepared.

FIG. 4C illustrates an artificial nucleic acid that includes a linking segment containing an affinity binding pair.

FIG. 4D illustrates a TSC composed of artificial nucleic acids that include linking segments containing affinity binding pairs.

FIG. 5 illustrates an exemplary approach to control the length of TSCs.

FIG. 6 illustrates a TSC in which each of the nucleic acid tethers shown contains a unique, identifiable sequence tag.

FIG. 7 illustrates a method of the invention in which TSCs are used to treat target DNA before subsequently obtaining the nucleotide sequence of the target DNA.

FIG. 8 illustrates the interaction of a TSC with a target DNA in order to initiate transposition of DNA at distal sites on the target DNA.

FIG. 9 illustrates the transposition of complementary subsequences, designated here as Y and Y′, from a TSC into distal sites on a target DNA molecule.

FIG. 10 illustrates the further fragmentation and adaptation of a target DNA treated with a TSC. The identifiable sequence tags Y and Y′ can be used to determine that the resulting library molecules arose from a tandem transposition mediated by transposases in TSCs.

FIG. 11 illustrates the use of identifiable sequence tags in sequencing target DNAs (e.g., DNA libraries) from a mixture of two distinct sources. DNA targets are treated with TSCs to infer pairs of mutations that arise from individual DNA molecules in that original target DNA mixture.

FIG. 12 illustrates that TSCs can be used to phase or link regions via a series of multiple transposition events in the target nucleic acid.

FIG. 13 illustrates a DNA molecule that is bound or wrapped around a structure such as a histone or nucleosome. This approach can be used to generate paired transposition events on distal sites of a DNA target in an ordered manner.

FIG. 14 illustrates a target DNA that has been treated with a DNA condensing agent and then bound by TSCs. The condensing agent increases the likelihood that distal sites within the target DNA are in close proximity to one another.

FIG. 15 illustrates insertion of a TSC at two distal sites of the pUC19 plasmid (see Example 2). The left panel shows a sequencing read obtained following treatment of pUC19 with a Tn5-MuA TSC. The structure of the inserted TSC is shown above the sequence read. The sequencing read is mapped to the location of the insertion sites on the pUC19 plasmid (right panel).

### DETAILED DESCRIPTION

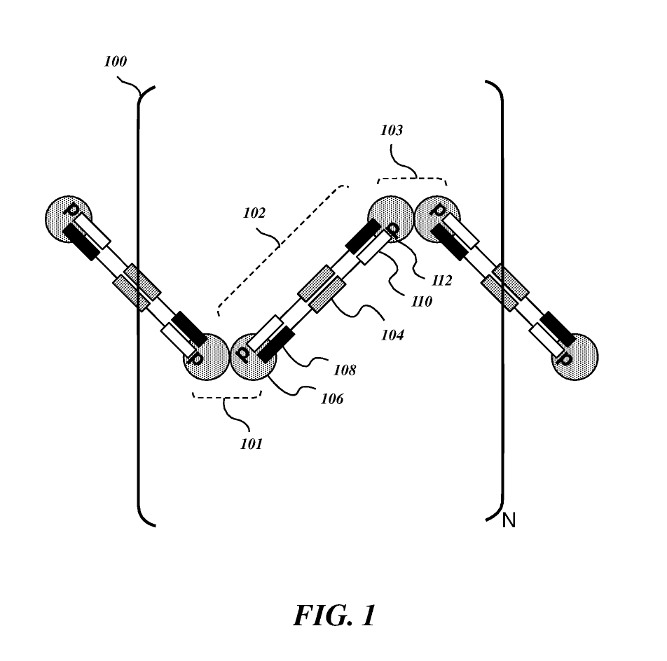

The invention provides, inter alia, tethered synaptic complexes (TSCs), nucleic acids, molecular constructs, synaptic complexes (e.g., ligatable synaptic complexes), TSC-modified libraries, and methods of use thereof. Below, specific compositions and methods encompassed by the present invention are described, examples and representative embodiments of which are shown in FIG. 1 to FIG. 15. These examples and embodiments should not be interpreted or construed as representing all possible embodiments or modifications of the claimed methods and compositions.

In general, the invention provides compositions that include a plurality of synaptic complexes. In one aspect, the compositions of the invention include the TSCs described herein, which we developed to allow multiple, distinct transposition events resulting in the insertion of known nucleic acid (e.g., DNA) cargo molecules (e.g., identifiable sequence tags) into sites within a target nucleic acid (e.g., DNA) that are separated by hundreds, thousands, or even millions of base pairs. In another aspect, the invention features methods of using the compositions described herein (e.g., TSCs) to obtain a library of nucleic acid (e.g., DNA) molecules from an original nucleic acid source. Such libraries can be used to determine the sequence of a template nucleic acid of interest (e.g., a genome). The methods can preserve and make readable information from two or more shorter subsequences on each library molecule originating from two potentially distal regions on the same original nucleic acid (e.g., DNA) molecule.

The compositions (e.g., TSCs) and methods described herein can be used in a wide variety of sequencing applications, particularly those in which incorporation of defined nucleic acid sequences (e.g., identifiable sequence tag(s)) into a target nucleic acid (e.g., DNA) is desired. The inventive approach creates a more accurate and valuable view of full sequence information of long segments of nucleic acids (e.g., DNA) by connecting regions present on the same original DNA molecule. The compositions and methods can be used, for example, to obtain fully phased resolved sequence information and can overcome the length limitation imposed by most NGS instruments. The compositions and methods also improve the ability to assemble longer regions, resolve difficult repeat regions, phase complex heterozygotes, and accurately identify RNA splice isoforms, as detailed further below.

Referring to FIG. 1, a representative example of a tethered synaptic complex (TSC) 100 is shown.

Specifically, two synaptic complexes 101 and 103 are tethered by a linear, double-stranded nucleic acid molecule 102. The linear, double-stranded nucleic acid molecule 102 contains two transposase binding sites 108 and 110 at opposing termini, in addition to a linking segment 104 that is situated between the two transposase binding sites. A first transposase 106 is bound to TBS 108, and a second transposase 112 is bound to TBS 110. In the nomenclature of the invention, the nucleic acid 102 is a tether between the two synaptic complexes 101 and 103. As one of ordinary skill in the art would recognize, the drawings are meant to illustrate the manner in which the components of the TSCs can be configured but do not limit the secondary or tertiary structure the TSCs may assume in the course of their manufacture or use. Similarly, the drawings are marked in the usual manner to refer to certain elements. These markings (e.g., the dotted lines used to refer to the synaptic complexes and nucleic acid tether) are not meant to define any strict point of demarcation between the elements. TSC 100 can include any suitable number N of subunits; for example, N can range from 1 to 1000 (e.g., 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 200, 300, 400, 500, 600, 700, 800, 900, or 1000). In some instances, N is greater than 1000.

Still referring to FIG. 1, in some embodiments the transposase binding sites 108 and 110 can be or can include any TBS described herein and/or known in the art, for example, the preferred mosaic outside end (OE) binding site sequence for Tn5 transposase, or a biologically active variant thereof. In any embodiment, the 5′ termini of sequences 108 and 110 can have an exposed terminal phosphate group (p) that is generated by methods known in the art, including, for example, chemical synthesis, transposase strand cleavage, or treatment with a polynucleotide kinase.

An essential and distinguishing feature of the tethered synaptic complexes depicted in FIG. 1, versus other synaptic complex compositions that are currently known in the art, is that the nucleic acid molecule 102 is bound to transposases in two distinct synaptic complexes (i.e., transposase 106 is part of synaptic complex 101, while transposase 112 is part of synaptic complex 103), and thus potentially two distinct transposition events occur once the tethered synaptic complexes are presented with a target DNA.

Still referring to FIG. 1, in some embodiments, one or more (and up to all) of the nucleic acid molecules that in one or more tethered synaptic complexes can also include a nucleotide or other chemical segment, as exemplified by linking segment 104, within the linear, double-stranded nucleic acid molecule that tethers any two synaptic complexes. If consisting purely of a polynucleotide sequence, this linking segment can be, or can include, a random, semi-random, or non-random DNA identifiable sequence tag and/or one or more primer binding sites. In other embodiments, segment 104 can be modified, derivatized, substituted or altered to include chemical functionality beyond that commonly conveyed by conventional nucleotides. For example, a nucleotide or other chemical component of linking segment 104 can be biotinylated in order to bind streptavidin or a derivative thereof, or replaced by non-nucleic acid linkers such as a non-nucleic acid polymer, for example, a polyether (e.g., polyethylene glycol (PEG)).

Still referring to FIG. 1, in certain preferred embodiments, the linear double stranded nucleic acid molecules bound to transposases will further polymerize according to the tendencies of transposases to bind one another once bound to their respective recognition sites. It will be clear to one of ordinary skill in the art that TSC 100 can vary in length and have a novel functional utility. For example, once the linear double stranded nucleic acid molecules polymerize via the binding of transposases, the TSCs are capable of initiating a plurality of tethered transposition events on a target nucleic acid (e.g., DNA) present in the same solution. As one of ordinary skill in the art would recognize, the TSCs must include transposases that are capable of carrying out a transposition event on a given target nucleic acid (e.g., DNA) with which they are combined in the context of the present methods.

Still referring to FIG. 1, in some embodiments the TBSs 108 and 110 can be or can include the outside end (CTGACTCTTATACACAAGT (SEQ ID NO:1)) or inside end (CTGTCTCTTGATCAGATCT (SEQ ID NO:2) or the hyperactive, hybrid of the outside and inside ends (CTGTCTCTTATACACATCT (SEQ ID NO:3) for the Tn5 transposase, as disclosed in, for example, Bhasin et al., J. Biol. Chem. 274:37021-37029, 1999. In other embodiments, the TBSs can be any of the TBSs described herein. As noted, biologically active variants of these sequences (e.g., variants that are at least about 80%, 85%, 90%, 92%, 95%, 96%, 97%, 98%, or 99% identical to a referenced counterpart (e.g., SEQ ID NO:1, 2, or 3)) can also be employed.

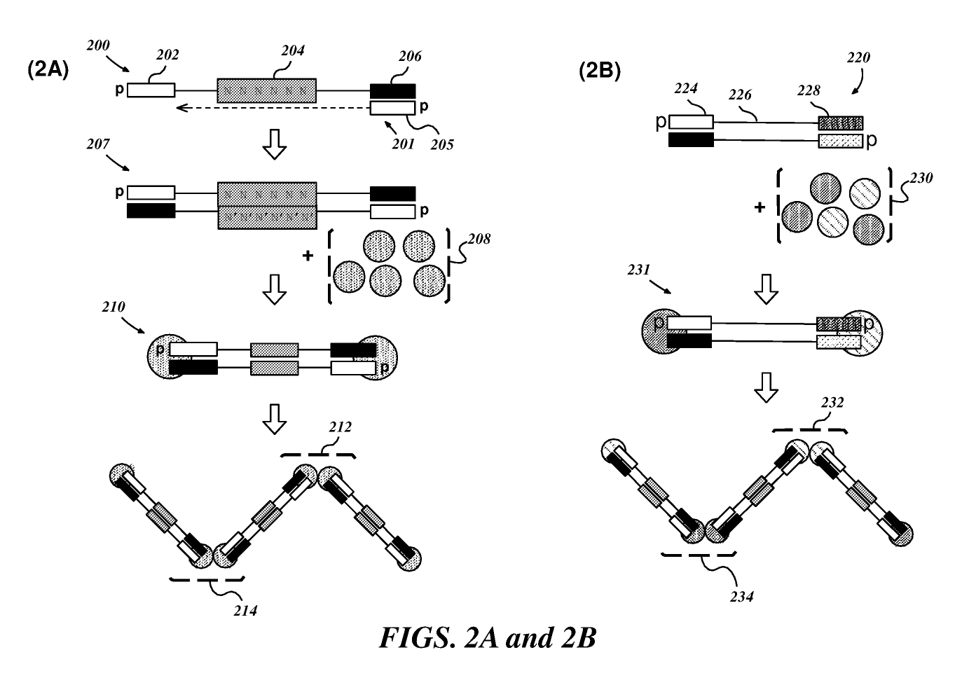

Referring now to FIG. 2A, an exemplary process by which TSCs can be made is shown. More specifically, a first nucleic acid 200 comprising the sequence of at least one TBS 206 (e.g., a terminal mosaic end outside sequence), is annealed to a second nucleic acid 201. The first and second nucleic acid can be synthesized and annealed according to standard techniques well known in the art. It follows that the sequences of the first and second nucleic acids can be complementary or sufficiently complementary that the nucleic acids will hybridize to one another under standard conditions (e.g., in the high salt, high temperature solutions considered to be moderately stringent). Alternatively, the first nucleic acid can be used as a template for extension of the second nucleic acid via polymerase-guided nucleotide incorporation, as illustrated in FIG. 2A by the dotted arrow. The first nucleic acid 200 includes the sequence of an identifiable sequence tag 204, which can be flanked by one or more primer binding sites. In certain preferred embodiments, nucleic acids 200 and 201 have terminal 5′ phosphates (indicated by “p” in FIG. 2A) to mediate their ligation to a target nucleic acid (e.g., a target DNA such as a chromosome). After a linear, double-stranded nucleic acid molecule 207 is formed, it can be combined with a plurality of transposases 208 in solution, under conditions that promote the binding of the transposases to one or both of the TBSs 206 and the TBS that will form at 202 on the linear, double-stranded nucleic acid molecule 207. The resulting subunit 210 includes a first transposase bound to TBS 202, a second transposase bound to TBS 206, and an intervening linking segment that includes identifiable sequence tag 204. The transposases that are members of the plurality of transposases 208 are capable of oligomerizing; however, the first transposase and the second transposase bound to subunit 210 do not oligomerize, for example, because of the intervening linking segment, which may have a length, sequence, and/or modifications that impair oligomerization. In FIG. 2A, each transposase is depicted as being identical. However, it is to be understood that the transposases could be, for example, biologically active variants (e.g., a mutated or artificial variant of the corresponding naturally occurring transposase) that remain capable of oligomerizing. Molecular construct 210 can oligomerize or polymerize to form TSCs via oligomerization of the transposases at each TBS to transposases bound to other subunits to form synaptic complexes 212 and 214, which are tethered by the linear, double-stranded nucleic acid molecule 207.

Exemplary methods of preparing TSCs having more than one type of transposase are illustrated in FIG. 2B. Linear, double-stranded DNA 220 includes TBSs 224 and 228 separated by an intervening linking segment 226. TBSs 224 and 228 each bind to transposases that cannot bind each other (for example, because they are different types of transposases). Linear, double-stranded DNA 220 can be combined with a mixture of transposases 230 that includes transposases that bind to one of TBS 224 and TBS 228 (indicated by transposases with solid grey fill and diagonal dashed lines, respectively). Binding of transposases to both TBSs results in subunit 231. As one non-limiting example of a molecular construct 231, one transposase is a Tn5 transposase and the other is a Mu transposase. The Tn5 transposase may bind to its TBS as a monomer, and the Mu transposase may bind to its TBS as a dimer.

Molecular construct 231 is also capable of oligomerizing or polymerizing to form TSCs via oligomerization of the transposases at each TBS to transposases bound to other subunits to form synaptic complexes 232 and 234.

Still referring to FIGS. 2A and 2B, the identical transposases in subunit 210, which form identical synaptic complexes within the TSCs of FIG. 2A, can be compared with the non-identical transposases in the subunit 231, which form non-identical synaptic complexes 232 and 234 within the TSCs of FIG. 2B.

Single stranded oligonucleotides such as those represented by 200 and 201, linear, double-stranded nucleic acids such as those represented by 207 and 220, subunits such as those represented by 210 and 231, as well as TSCs such as those containing the identical and non-identical synaptic complexes shown in FIGS. 2A and 2B are all compositions within the scope of the present invention. In any embodiment, a given transposase can be naturally occurring or a biologically active variant thereof (e.g., a mutated or artificial variant of the corresponding, naturally occurring transposase). A biologically active variant may specifically bind the same site on a target DNA as the corresponding, naturally occurring transposase, or the variant may be engineered specifically to bind a different site. For example, the TSCs can include one or more different variants of Tn5 that have been engineered to produce different binding site preferences. It may be advantageous to create TSCs from subunits having different/non-identical transposases at opposite termini of the linear, double-stranded nucleic acid molecules, particularly at each end of the TSCs, as this may help minimize or prevent non-productive DNA looping or circularization.

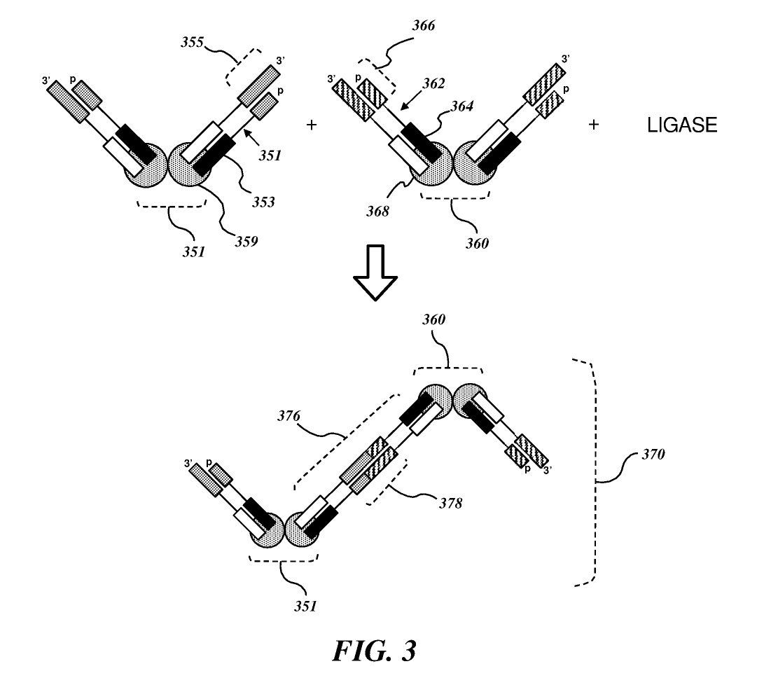

Another exemplary method of preparing TSCs is shown in FIG. 3. Ligatable synaptic complex 351 includes transposase 359, which is bound to TBS 353 of linear, double-stranded DNA 351. Linear, double-stranded DNA 351 also includes sticky end 355, which includes a 3′ overhang. Ligatable synaptic complex 360 includes transposase 368, which is bound to TBS 364 of linear, double-stranded DNA 362.

Linear, double-stranded DNA 362 also includes sticky end 366, which includes a 3′ overhang that is compatible with sticky end 355 of linear, double-stranded DNA 351 due to sequence complementarity of the overhangs. Addition of a ligase under suitable conditions to ligatable synaptic complexes 351 and 360 results in ligation of linear, double-stranded DNAs 351 and 362 at sticky ends 355 and 366, thereby preparing TSC 370. In TSC 370, synaptic complexes 351 and 360 are tethered by the linear, double stranded DNA 376, which results from ligation of linear, double stranded DNAs 351 and 362. Linking segment 378 of linear, double-stranded DNA 376 includes nucleic acid sequences from sticky ends 355 and 366, which may be used as an identifiable sequence tag. The exemplary method shown in FIG. 3 can be used to create TSCs having synaptic complexes containing the same type of transposases, as shown, or can be used to ligate synaptic complexes composed of different/non-identical transposases.

For example, a synaptic complex containing an oligomerized pair of Tn5 transposases can be ligated to a synaptic complex containing an oligomerized tetramer of Mu transposases. Additional ligatable synaptic complexes can be ligated to TSC 370 to form TSCs having any desired number of synaptic complexes.

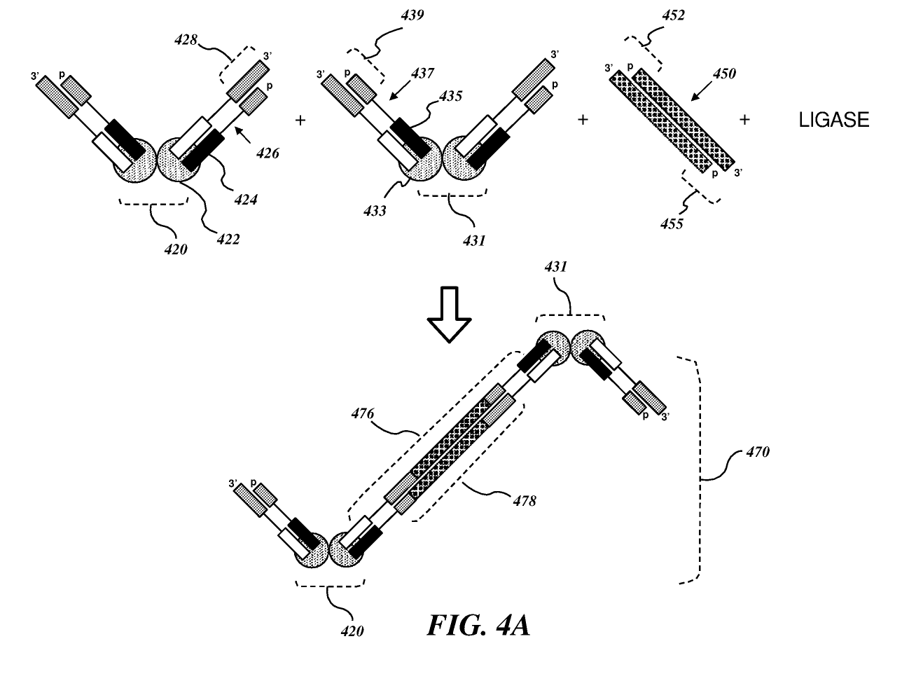

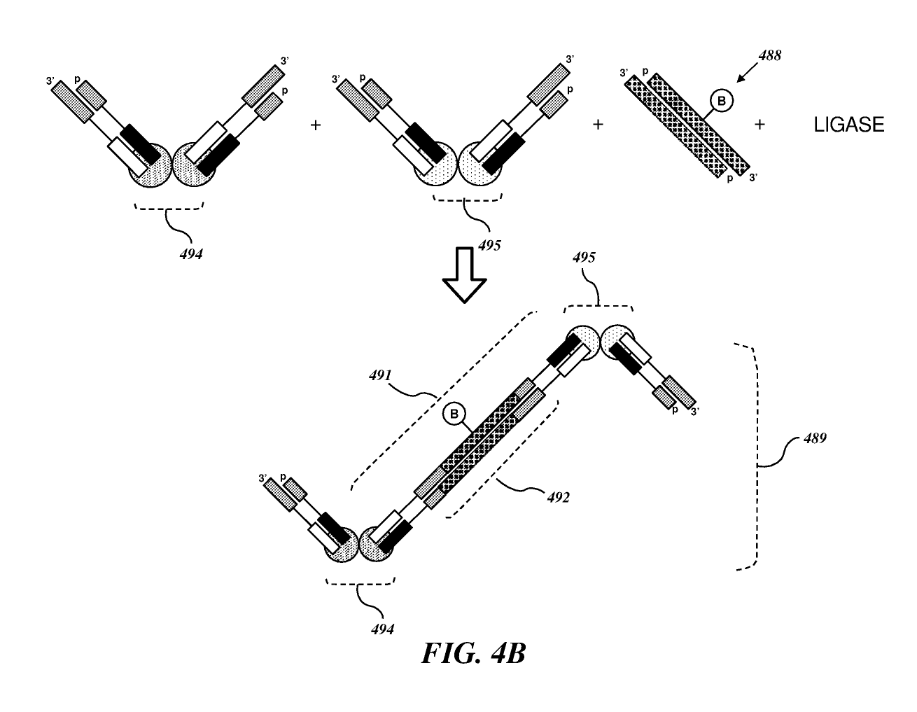

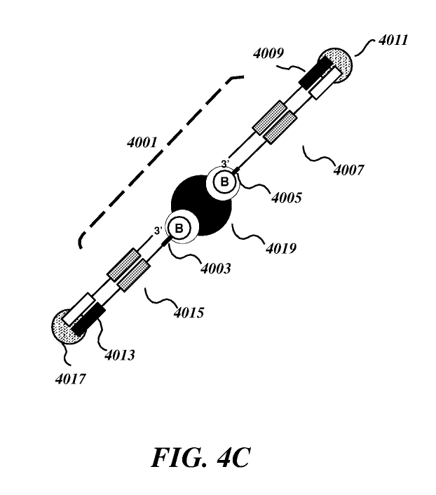

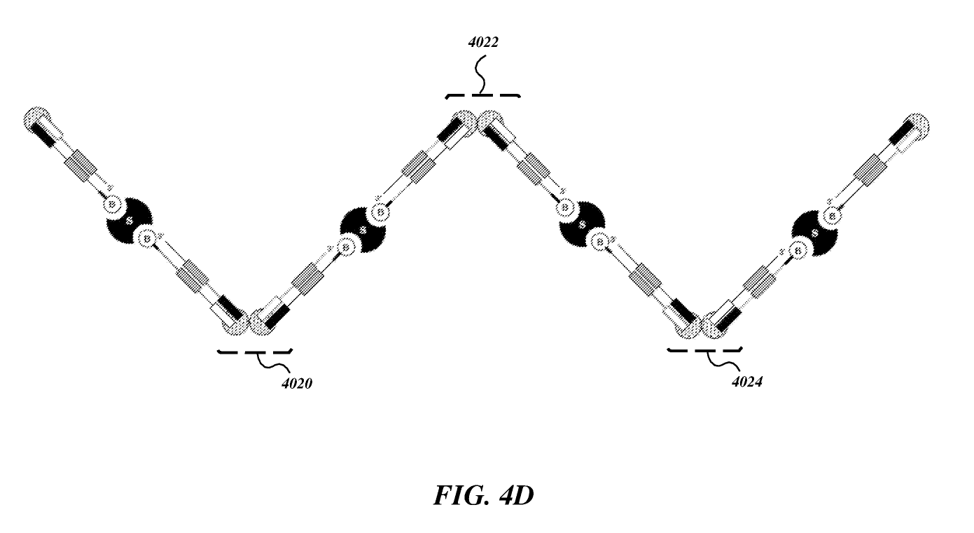

FIGS. 4A and 4B show additional exemplary methods of preparing TSC. The approach shown in FIGS. 4A and 4B involves ligation of ligatable synaptic complexes, similar to the approach shown in FIG. 3. In FIG. 4A, ligatable synaptic complexes are ligated to a linking segment nucleic acid, which can be used to form TSCs that include nucleic acids having desired linking segment nucleic acid sequences (e.g., identifiable sequencing tags). As shown in FIG. 4B, this approach can be used to introduce linking segments that are modified, derivatized, substituted, or altered to include chemical functionality beyond that commonly conveyed by conventional nucleotides, for example, a biotinylated linking segment.

Referring now to FIG. 4A, ligatable synaptic complex 420 includes transposase 422, which is bound to TBS 424 of linear, double-stranded DNA 426. Linear, double-stranded DNA 426 also includes sticky end 428, which includes a 3′ overhang. Ligatable synaptic complex 431 includes transposase 433, which is bound to TBS 435 of linear, double-stranded DNA 437. Linear, double-stranded DNA 437 also includes sticky end 439, which includes a 3′ overhang. Linking segment nucleic acid 450 includes a first sticky end 452, which has a 3′ overhang that is compatible with sticky end 428 due to sequence complementarity of the overhangs. Linking segment nucleic acid 450 also includes a second sticky end 455, which includes a 3′ overhang that is compatible with sticky end 439 due to sequence complementarity of the overhangs. Addition of a ligase under suitable conditions to ligatable synaptic complexes 420 and 431 results in ligation of linear, double-stranded DNA 426 to linking segment nucleic acid 450 at sticky ends 428 and 452, as well as ligation of linear, double-stranded DNA 437 to linking segment nucleic acid at sticky ends 439 and 455. The ligation reaction results in formation of TSC 470.

In TSC 470, synaptic complexes 420 and 431 are tethered by the linear, double stranded DNA 476, which results from ligation of linear, double stranded DNAs 426 and 437 to linking segment nucleic acid 450.

Linking segment 478 of linear, double-stranded DNA 476 includes nucleic acid sequences from sticky ends 428 and 439, as well as the nucleic acid sequence of linking segment nucleic acid 450, and may include, for example, an identifiable sequence tag, a primer binding site, a cleavage site, and/or other sequence elements. The exemplary method shown in FIG. 4A can be used to create TSCs having synaptic complexes containing identical transposases, as shown, or can be used to ligate synaptic complexes composed of different/non-identical transposases. For example, a synaptic complex containing an oligomerized pair of Tn5 transposases can be ligated to a synaptic complex containing an oligomerized tetramer of Mu transposases. Additional ligatable synaptic complexes can be ligated to TSC 470 to form TSCs having any desired number of synaptic complexes.

Referring now to FIG. 4B, a similar approach as illustrated in FIG. 4A is used. However, in this example, linking segment nucleic acid 488 is biotinylated (B). Following ligation of ligatable synaptic complexes 494 and 495 to linking segment nucleic acid 488, the resulting TSC 489 includes linear, double-stranded DNA 491 includes linking segment 492, which is biotinylated (B). In this example, ligatable synaptic complexes 494 and 495 contain different types of transposases, for example, Tn5 and Mu, respectively. It is to be understood that in other instances, ligatable synaptic complexes 494 and 495 may both contain the same type of transposase. Additional ligatable synaptic complexes can be ligated to TSC 489 to form TSCs having any desired number of synaptic complexes.

Referring now to FIG. 4C, a still further approach to prepare TSCs using linking segments containing affinity binding pairs is shown. Artificial nucleic acid 4007 contains TBS 4009 at its first end, and is biotinylated (B) at second end 4005. Similarly, artificial nucleic acid 4015 contains TBS 4013 at its first end, and is biotinylated (B) at second end 4003. Artificial nucleic acids 4007 and 4015 are non-covalently linked by binding of the biotin moieties at ends 4005 and 4003 to streptavidin moiety 4019 after incubation under suitable conditions. This interaction results in formation of linking segment 4001. Transposases 4017 and 4011 can bind to TBSs 4013 and 4009, respectively.

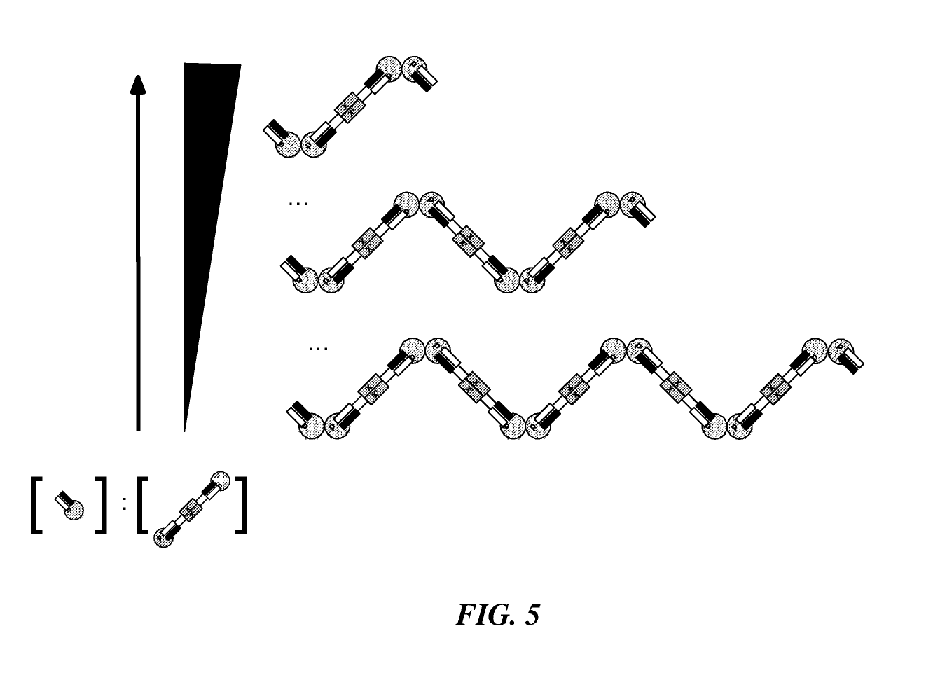

FIG. 4D illustrates an exemplary TSC formed by non-covalent linkage of artificial nucleic acids by linking segments that contain affinity binding pairs, such as biotin-biotin binding protein (e.g., biotin (B)-streptavidin(S)), as shown in FIG. 4C. The resulting TSC includes synaptic complexes 4020, 4022, and 4024. FIG. 5 illustrates that the length of TSCs, measured as the number of synaptic complexes that are co-connected via a series of oligomerized transposases and their TBS-containing tethering nucleic acid molecules, can be varied by controlling the stoichiometric ratio of tethering TBS-containing nucleic acid molecules with ordinary (non-tethering) TBS-containing nucleic acid molecules. Non-tethering nucleic acid molecules act to “terminate” the creation of longer polymers of TSCs by outcompeting the oligomerization of transposases bound to tethering TBS-containing nucleic acid molecules.

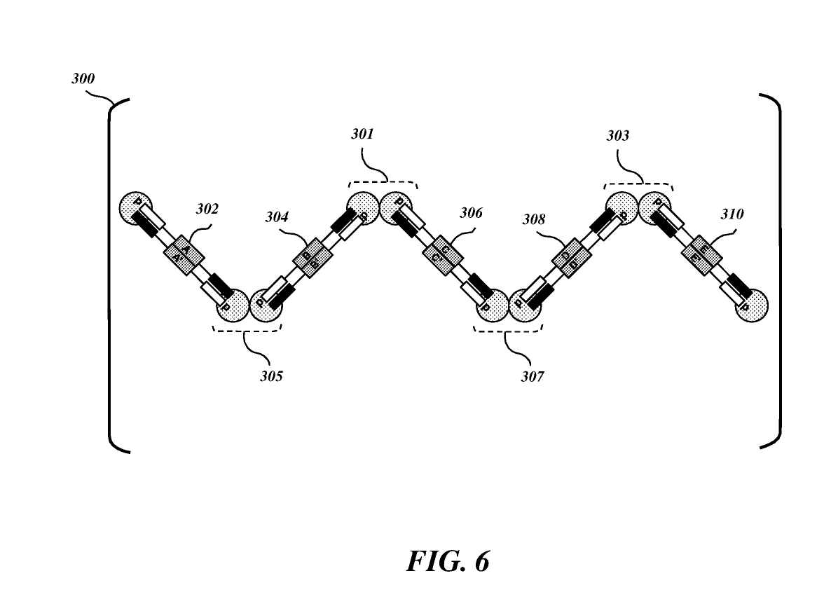

FIG. 6 illustrates TSC 300 including the synaptic complexes 301, 303, 305 and 307, where the synaptic complexes of the TSC are connected via nucleic acid molecules with internal subsequences 302, 304, 306, 308 and 310. In some embodiments, the subsequences within each polymeric subunit are degenerate random, semi-random or non-random identifiable sequence tags, or one or more primer binding sites. In various embodiments, the identifiable sequence tags may be identical or non-identical between different subunits of the TSCs.

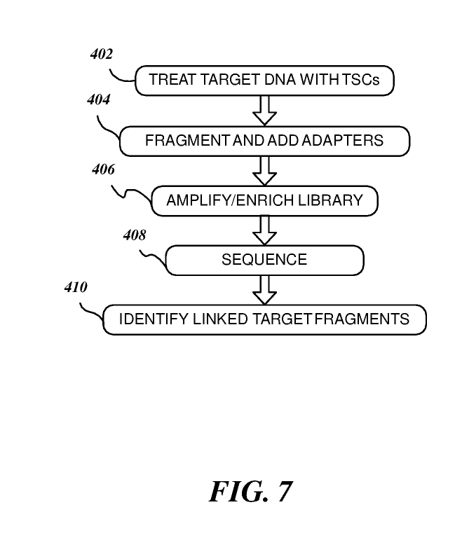

Referring now to methods encompassed by the present invention, FIG. 7 shows a schematic illustration of steps that represent a broadly applicable embodiment of the present invention.

In FIG. 7, there is first shown a step 402 where a target DNA is treated with the prepared tethered synaptic complex composition. The target DNA can be any source where the ascertainment of the sequence of the target DNA is of interest. For example, the target DNA can be the genomic DNA from an organism, such as a human, or be a mixture of DNA molecules from more than one organism. Under conditions that are taught by the present invention, the treatment of the target DNA by the TSC composition causes the transposition of cargo DNA into distal sites on individual target molecules of the target DNA. In some cases, the identifiable sequence tags on the transferred DNA are later used to identify the TSC subunit responsible for catalysis of distal transposition events within the original tethered synaptic complex.

Still referring to FIG. 7, there is next shown a fragmentation and adapter addition step (also called “shotgun adaptation”) step 404 in which the target DNA, having been treated with TSC, is then modified or processed to incorporate, by any of several methods known in the art, a specified polynucleotide tag sequence, also commonly called a DNA barcode, into DNA fragment or fragments produced by shotgun fragmentation and adaptation. The shotgun fragmentation and adaptation can be accomplished, for example, by random shearing and adapter ligation, or transposase adaptation (i.e., tagmentation), or by any similar method known in the art. Following the shotgun adaptation step, an amplification and enrichment step 406 is then used specifically to amplify products that contain transposable nucleic acid sequences that originate from the treatment of target DNA with TSCs in step 402 and the subsequent shotgun adaptation step 404. The products of this amplification can then be further processed (e.g., purified or size-selected) by conventional means and then subjected to highly parallelized DNA sequencing (e.g., Illumina MiSeq™, NextSeq™ 500, HiSeq™, or ION TORRENT™ sequencing) in step 408 to obtain a large multiplicity of raw sequencing reads that represent the derived library of interest.

Still referring to FIG. 7, we show an assembly and/or analysis step 410 in which the derived reads from the library of the inventive method are analyzed to obtain information regarding the sequence composition of the original DNA sample. Of particular utility with respect to the inventive method is the use of information encoded by identifiable sequence tags that have been incorporated into the TSCs of the present invention. In some embodiments, the identifiable sequence tags can be used to link distinct sequencing reads obtained among a large multiplicity of reads has having originated from distal locations (e.g., many bases away) on the same DNA molecule among many DNA molecules present in the original sample. This information can be used, for example, to determine whether two mutations present in the same sample originate from the same copy of a chromosome of a diploid DNA sample, for example detecting linkages between mutations in human target DNA that are too far apart to observe in a single sequencing read. In other aspects this information can also be used computationally to assist the assembly of a large multiplicity of raw sequencing reads into long contiguous derived sequences.

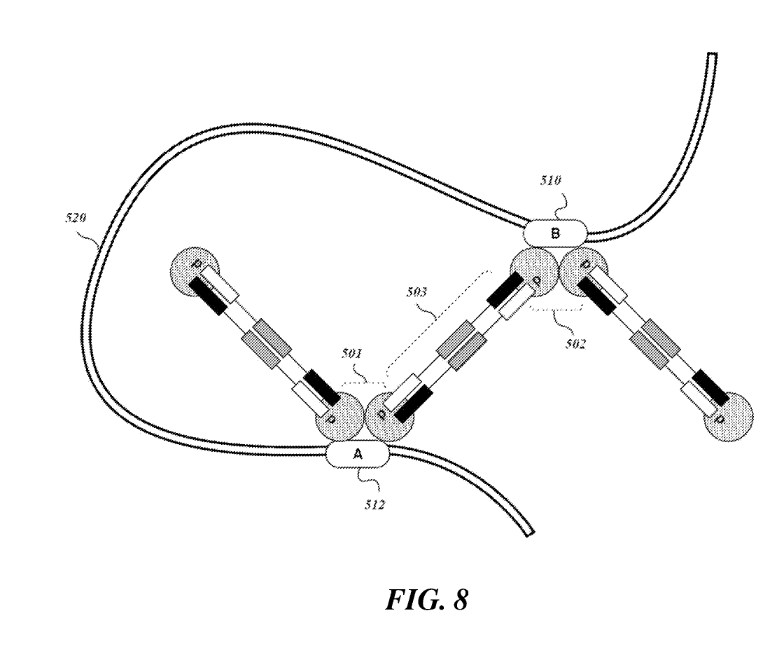

Referring now to FIG. 8, there is shown an illustration of the method by which TSCs can be used to treat a target DNA 520 and thereby infer the linkage between two distal sites on the same DNA molecule. Specifically, there is shown a TSC that includes two synaptic complexes 501 and 502 tethered by a nucleic acid molecule 503. Synaptic complexes 501 and 502 are shown as respectively contacting two distal sites A (512) and B (510). The two distal sites could represent, for example, two subsequences on a chromosome, separated by 1,000 bp, or 10,000 bp, or 100,000 bp, or more. For the purposes of illustration, the length of the target DNA molecule in FIG. 8 is not shown to scale.

One particularly useful aspect of the invention is the enhanced tendency for some embodiments of TSCs to act on the same DNA molecule, even in the presence of a large plurality of different DNA molecules present in a mixture. For example, using the illustration of FIG. 8, once presented with either of the two sites 510 and 512 situated on the same DNA molecule present in a complex mixture of a plurality of DNA molecules, the contact and binding of either of synaptic complexes 501 or 502 of the TSC will be highly entropically constrained and thus favor the contact and binding of the same TSC to another site present on the same target DNA molecule.

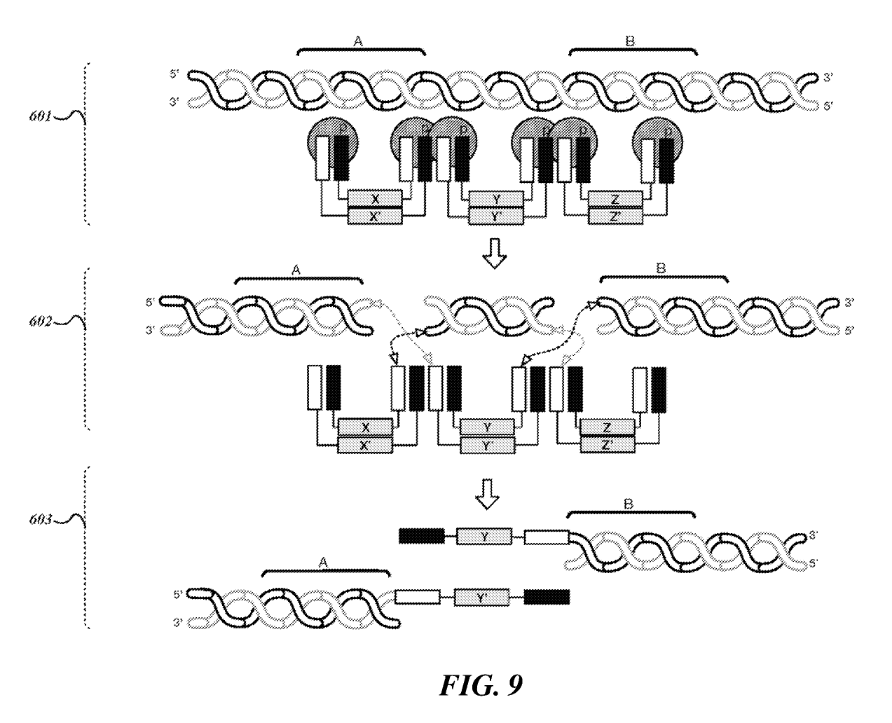

Referring now to FIG. 9, a process is shown whereby pairs of linked and identifiable sequence tags are transferred via TSCs onto a target. As illustrated in FIG. 9, a TSC is shown in step 601, wherein two synaptic complexes are tethered by a nucleic acid segment that contains a sequence tag Y and its reverse complement Y′. Step 602 shows the transposition and covalent attachment of specific strands from the TSC to strands from regions A and B of a target DNA. The results of this transposition are shown in step 603, where identifiable sequence tags Y and Y′ are now covalently attached to strands of A and B. For simplicity, transposition products which may result from ligation of the nucleic acid segments containing the X or Z′ identifiable sequence tags to the excised DNA fragment between regions A and B are not shown. It is to be understood that each identifiable sequence tag that is transposed into the target DNA sequence can be used for determining the linkage of the DNA fragments that result from the transposition reaction.

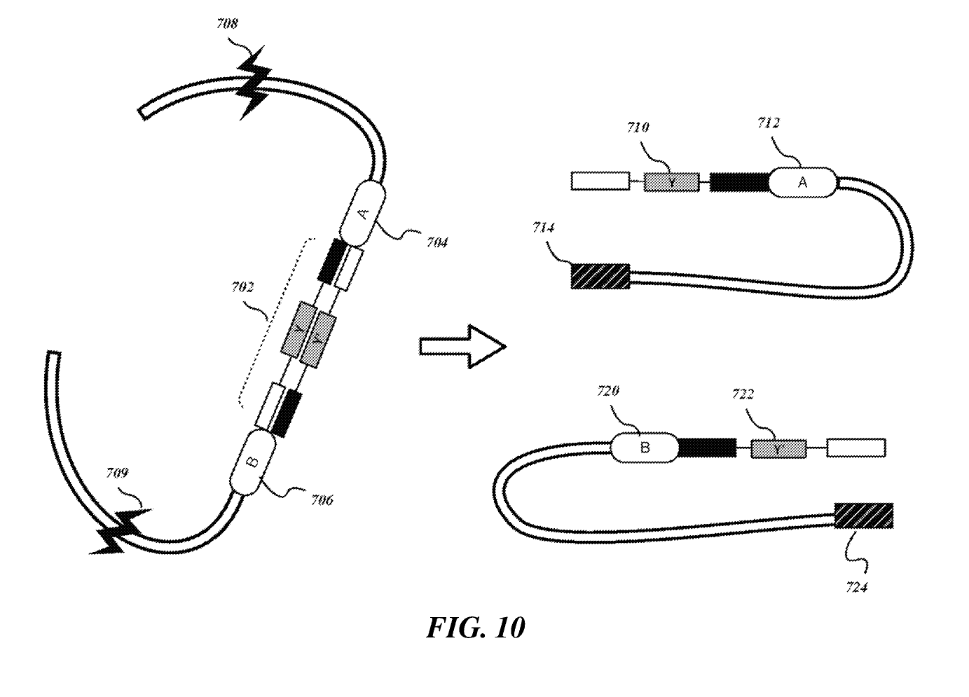

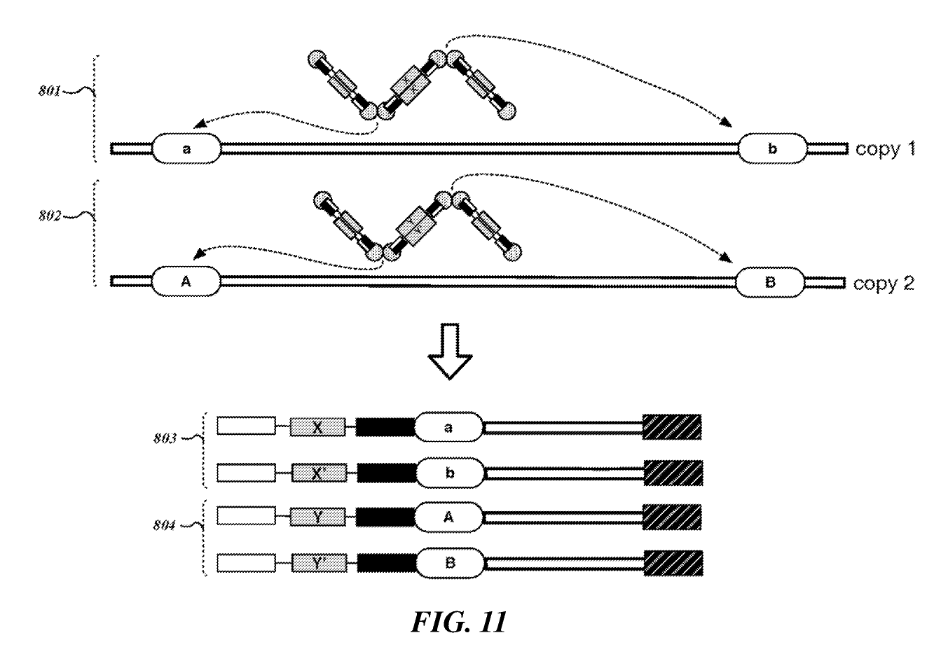

Referring now to FIG. 10, an example of products of the transposition reaction caused by TSCs such as those embodied by FIG. 8 and FIG. 9 is shown. The structure shown in the left panel of FIG. 10 may be generated, for example, by performing optional steps of fill-in (using DNA polymerase) and/or ligation (using DNA ligase) on the products shown in step 603 of FIG. 9, which are able to hybridize due to sequence complementarity. Specifically, the distal sites A and B (704 and 706) are now connected via the nucleic acid 702. Furthermore, in this embodiment, the nucleic acid 702 further contains an identifiable sequence tag that becomes attached to the transposition target DNA sites 704 and 706.

Upon shotgun adaptation, the structure on the left side of FIG. 10 is converted to two distinct fragments by fragmentation at sites 708 and 709, and addition of adapter sequence at the site of fragmentation. As a result, as shown on the right side of FIG. 10, two fragments are generated, one consisting of an identifiable sequence tag Y (710) connected to region A (712) of the original DNA molecule, and the other consisting of the reverse complement of this sequence tag Y′ (722) connected to region B (720) of the original DNA molecule. Both of the two fragments also contain a common shotgun adapter sequence (714 and 724). In this example, the resulting fragments, when sequenced, can be used to link together the two regions A and B as originating from the same DNA molecule by virtue of sharing unique identifiable sequence tags (Y and Y′) that can be computationally matched among a large number of diverse tags.

Referring now to FIG. 11, there is shown an example whereby TSCs can be used resolve the potentially unknown phasing relationship in a diploid sample between alternate alleles at two linked loci.

Specifically, in 801 there is shown a copy of one region where two mutations “a” and “b” are connected via tandem transposition events of TSCs, whereas in the corresponding other copy 802 the mutations “A” and “B” are connected via tandem transposition events of a differently labeled TSC.

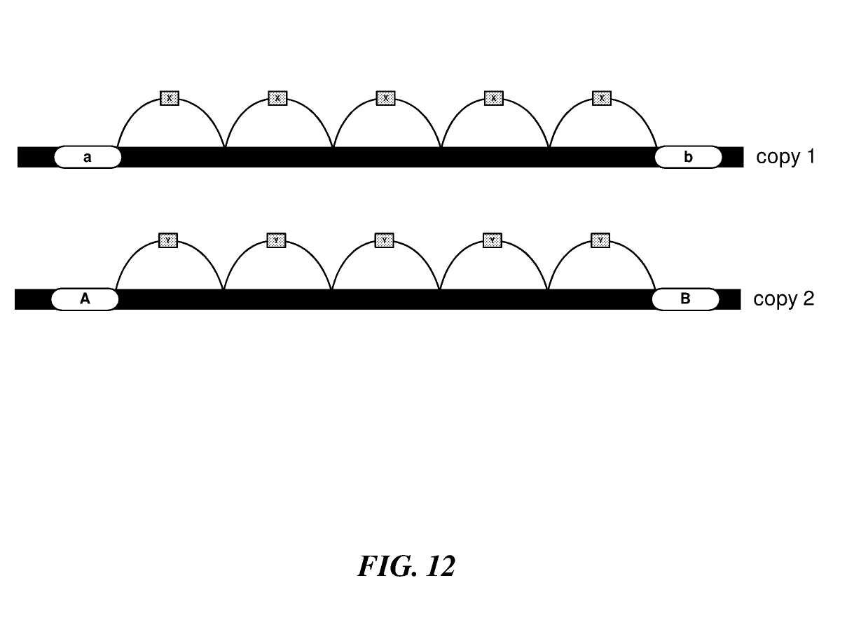

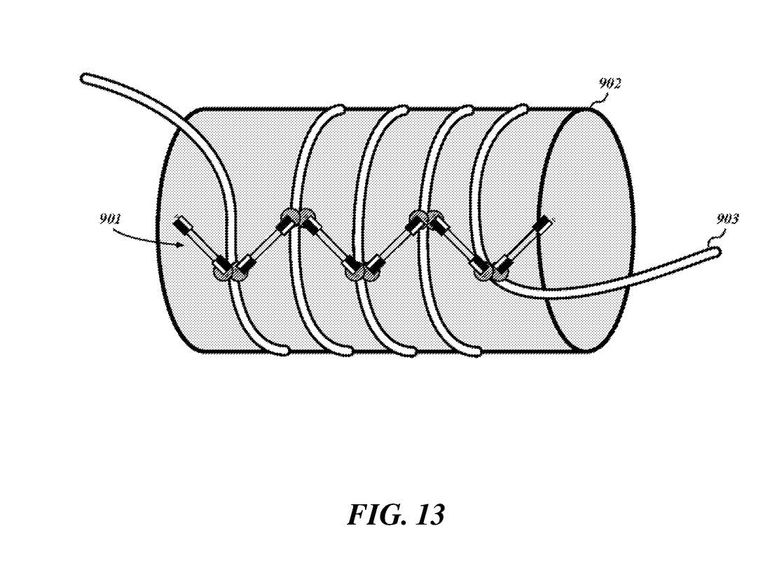

Referring now to FIG. 12, there is shown an example whereby TSCs can be used to phase or link regions via a series of multiple transposition events. The multimeric nature of the TSCs can result in multiple transposition events in the same target nucleic acid molecule, which can be used for phasing of alternate alleles at two linked loci.

### Compositions of the Invention

The invention provides TSCs, as well as intermediates formed during preparation of TSCs. The invention further provides compositions that include artificial nucleic acids, as well as molecular constructs and TSCs that contain them. In general, the artificial nucleic acids of the invention include one or more TBSs. In some embodiments, the artificial nucleic acids include a TBS at each terminus separated by an intervening linker segment. Molecular constructs of the invention include artificial nucleic acids that are bound to transposase proteins. These molecular constructs can be subunits of TSCs, which can be higher-order oligomers and polymers.

The invention provides TSCs that include synaptic complexes tethered by artificial nucleic acids having TBSs at each terminus. For example, in some instances, a TSC includes a first artificial nucleic acid that includes a first end including a first transposase binding site (TBS), a second end including a second TBS, and a linking segment disposed between the first TBS and the second TBS; a second artificial nucleic acid including a first end including a first TBS; a third artificial nucleic acid including a first end including a first TBS; a first synaptic complex including a first pair of oligomerized transposases, the first pair including (or consisting of) a first transposase and a second transposase, wherein the first transposase is bound to the first TBS of the first artificial nucleic acid, and the second transposase is bound to the first TBS of the second artificial nucleic acid; and a second synaptic complex including a second pair of oligomerized transposases, the second pair including (or consisting of) a third transposase and a fourth transposase, wherein the third transposase is bound to the second TBS of the first artificial nucleic acid, and the fourth transposase is bound to the first TBS of the third artificial nucleic acid.

The linking segment can include a nucleic acid (e.g., DNA). The nucleic acid can be at least partially single-stranded. In some instances, the nucleic acid is fully double-stranded. The linking segment may include terminal nucleotides that form phosphodiester bonds with the first TBS and the second TBS. In some instances, the linking segment further may include one or more additional elements such as an identifiable sequence tag (IST), a primer binding site, a cleavage site, or a chemical modification. The IST may be a random IST, a semi-random IST, or a non-random IST. The cleavage site may be a restriction endonuclease recognition site or a nickase site.

Any of the linking segments disclosed herein may include an affinity binding pair. The affinity binding pair may include biotin-biotin binding protein (e.g., biotin-streptavidin or biotin-avidin), ligand-receptor, antigen-antibody or antigen binding fragment, hapten-anti-hapten, or immunoglobulin (Ig) binding protein-lg. In some instances, the affinity binding pair includes biotin-streptavidin or biotin-avidin.

The streptavidin or avidin may bind, for example, only one or two biotin molecules. In some instances, the affinity binding pair may include a first affinity component that binds to two second affinity components, where one second affinity component is linked to the first end of the first artificial nucleic acid, and the other second affinity component is linked to the second end of the first artificial nucleic acid.

In other instances, the affinity binding pair includes a first affinity component that binds a second affinity component, where the first affinity component is linked to the first end of the first artificial nucleic acid, and the second affinity component is linked to the second end of the first artificial nucleic acid.

Any of the linking segments disclosed herein may include a covalent bond resulting from a conjugation reaction. The covalent bond may result from any suitable conjugation reaction, for example, a cycloaddition, amide or thioamide bond formation, a pericyclic reaction, a Diels-Alder reaction, sulfonamide bond formation, alcohol or phenol alkylation, a condensation reaction, disulfide bond formation, and a nucleophilic substitution. In some instances, the cycloaddition is an azide-alkyne Huisgen cycloaddition (e.g., a copper(I)-catalyzed azide-alkyne cycloaddition (CuAAC) or a strain-promoted azide-alkyne cycloaddition (SPAAC)).

The artificial nucleic acid can be any suitable length. For example, in some instances, the artificial nucleic acid is about 20 to about 10,000 base pairs (bp) long. For example, the artificial nucleic acid can be about 20, about 25, about 30, about 35, about 40, about 45, about 50, about 55, about 60, about 65, about 70, about 75, about 80, about 85, about 90, about 100, about 120, about 140, about 160, about 180, about 200, about 225, about 250, about 275, about 300, about 400, about 500, about 1000, about 2000, about 5000, or about 10,000 bp in length. In some instances, the artificial nucleic acid is about 100 to about 250 bp long (e.g., about 100 to about 250 bp long, about 100 to about 200 bp long, about 100 to about 150 bp long, about 100 to about 125 bp long, about 125 to about 250 bp long, about 125 to about 225 bp long, about 125 to about 200 bp long, about 125 to about 175 bp long, about 125 to about 150 bp long, about 150 to about 250 bp long, about 150 to about 225 bp long, about 150 to about 200 bp long, or about 150 to about 175 bp long). In some instances, the artificial nucleic acid is about 150 to about 200 bp long. For example, the artificial nucleic acid may be about 175 bp long.

A linking segment can have a mass of about 15 fg or less, about 14 fg or less, about 13 fg or less, about 12 fg or less, about 11 fg or less, about 10 fg or less, about 9 fg or less, about 8 fg or less, about 7 fg or less, about 6 fg or less, about 5 fg or less, about 4 fg or less, about 3 fg or less, about 2 fg or less, about 1 fg or less, about 1×10⁻¹⁶ g or less, about 1×10⁻¹⁷ grams or less, about 1×10⁻¹⁸ grams or less, about 1×10⁻¹⁹ grams or less, or about 1×10⁻²⁰ grams or less. For example, in some instances, the linking segment has a mass of about 1×10⁻²⁰ grams to about 15 fg (e.g., about 1×10⁻²⁰ grams to about 15 fg, about 1×10⁻²⁰ grams to about 10 fg, about 1×10⁻²⁰ grams to about 5 fg, about 1×10⁻²⁰ grams to about 1 fg, about 1×10⁻²⁰ grams to about 1×10⁻¹⁶ g, about 1×10⁻²⁰ grams to about 1×10⁻¹⁷ g, about 1×10⁻²⁰ grams to about 1×10⁻¹⁸ g, or about 1×10⁻²⁰ grams to about 1×10⁻¹⁹ g).

In some instances, the transposases of the first pair and the second pair may be of different types, for example, Tn5 and Mu. In other instances, the transposases of the first pair and the second pair may be of the same type, for example, Tn5.

In some instances, at least one transposase of the first pair and/or the second pair can be operably linked to a targeting moiety. For example, a transposase in a given synaptic complex can be operably linked to a targeting moiety. The targeting moiety may be, for example, a polypeptide including a DNA-binding domain (DBD) or an RNA-guided endonuclease. In some instances, the DBD may be, for example, a zinc finger motif or a transcription activator-like (TAL) effector. The RNA-guided endonuclease may be, for example, Cas9, Cpf1, C2c2, or a biologically active variant thereof. The biologically active variant may be a nuclease-deficient variant.

In any of the preceding TSCs, the second artificial nucleic acid or the third artificial nucleic acid may further include a second end, wherein the second end is a ligatable end (e.g., a sticky end). For example, in some instances, the second artificial nucleic acid further includes a second end, wherein the second end is a ligatable end (e.g., a sticky end). In some instances, the third artificial nucleic acid further includes a second end, wherein the second end is a ligatable end (e.g., a sticky end). In some instances, the second artificial nucleic acid and the third artificial nucleic acid each further includes a second end, wherein the second end is a ligatable end (e.g., a sticky end).

In other instances, the second artificial nucleic acid or the third artificial nucleic acid may further include a component of a second affinity binding pair or a conjugating moiety. The conjugating moiety may be, for example, a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a sulfhydryl, a thiol, or a thioisocyanate group. Other conjugating groups are known in the art.

In another example, in any of the preceding TSCs, the second artificial nucleic acid may further includes a second end comprising a second TBS, and a linking segment disposed between the first TBS and the second TBS of the second artificial nucleic acid. The linking segment of the second artificial nucleic acid may include a second affinity binding pair or a covalent bond resulting from a conjugation reaction

In yet another example, in any of the preceding TSCs, the third artificial nucleic acid may further includes a second end comprising a second TBS, and a linking segment disposed between the first TBS and the second TBS of the second artificial nucleic acid. The linking segment of the third artificial nucleic acid may include a second affinity binding pair or a covalent bond resulting from a conjugation reaction

Any of the preceding TSCs may further include one or more additional synaptic complexes (e.g., 1-10,000 additional synaptic complexes), each additional synaptic complex including a pair of oligomerized transposases, and one or more additional artificial nucleic acids (e.g., 1-10,000 additional artificial nucleic acids), each additional artificial nucleic acid including a TBS at each end and an intervening linking segment, wherein each of the first synaptic complex, the second synaptic complex, and the one or more additional synaptic complexes is tethered to at least one other synaptic complex of the TSC, and wherein any two synaptic complexes of the TSC are tethered by binding to TBSs at either end of the same artificial nucleic acid. In some instances, each artificial nucleic acid of the TSC includes an IST. The ISTs may be identical, or the TSC may include a plurality of different ISTs.

The invention further provides artificial nucleic acids that include a first end having a first TBS, a second end having a second TBS, and a linking segment disposed between the first TBS and the second TBS. In particular embodiments, upon binding of a first transposase to the first TBS and a second transposase to the second TBS, the first transposase does not oligomerize with the second transposase.

The linking segment may minimize or prevent the first transposase and the second transposase from oligomerizing when bound to the first TBS and the second TBS, respectively. This may be due to the length of the linking segment; for example, the length may be too short (or too long) to allow for oligomerization. In other instances, the linking segment may include a nucleic acid sequence or chemical modification that minimizes or prevents oligomerization. The linking segment may include a nucleic acid, which may be at least partially single-stranded or double-stranded. The linking segment may include terminal nucleotides that form phosphodiester bonds with the first TBS and the second TBS. In other instances, the linking segment may include a non-nucleotide chemical moiety (e.g., a non-nucleotide polymer, e.g., a polyether such as PEG), an amino acid, peptide, or polypeptide. Approaches for conjugating various chemical entities to nucleic acids are known in the art and described herein.

The linking segment may further include one or more additional elements. For example, the linking segment may include an IST, a primer binding site, or a cleavage site. The IST may be, for example, a random IST, a semi-random IST, or a non-random IST. Approaches for generating ISTs, such as barcodes, are known in the art. The cleavage site may be, for example, a restriction endonuclease recognition site or a nickase site. The linking segment may be any suitable length, for example about 20 bp to about 1000 bp or more in length, which may vary depending on the nature of the transposases intended for use with the artificial nucleic acid, as described herein. For example, the linking segment may be about 20, about 25, about 30, about 35, about 40, about 45, about 50, about 55, about 60, about 65, about 70, about 75, about 80, about 85, about 90, about 100, about 120, about 140, about 160, about 180, about 200, about 225, about 250, about 275, about 300, about 400, about 500, about 1000, about 2000, about 5000, or about 10,000 bp in length.

In some instances, the linking segment has a length in the range of between about 20 and about 5,000 bp, about 20 and about 2,000 bp, about 20 and about 1,000 bp, about 20 and about 900 bp, about 20 and about 800 bp, about 20 and about 700 bp, about 20 and about 700 bp, about 20 and about 600 bp, about 20 and about 500 bp, about 20 and about 400 bp, about 20 and about 300 bp, about 20 and about 200 bp, about 20 and 100 bp, about 20 and about 65 bp, about 50 and about 5,000 bp, about 50 and about 2,000 bp, about 50 and about 1,000 bp, about 50 and about 900 bp, about 50 and about 800 bp, about 50 and about 700 bp, about 50 and about 700 bp, about 50 and about 600 bp, about 50 and about 500 bp, about 50 and about 400 bp, about 50 and about 300 bp, about 50 and about 200 bp, about 50 and about 100 bp, about 50 and about 65 bp, about 100 and about 5,000 bp, about 100 and about 2,000 bp, about 100 and about 1,000 bp, about 100 and about 900 bp, about 100 and about 800 bp, about 100 and about 700 bp, about 100 and about 700 bp, about 100 and about 600 bp, about 100 and about 500 bp, about 100 and about 400 bp, about 100 and about 300 bp, about 100 and about 200 bp, about 200 and about 5,000 bp, about 200 and about 2,000 bp, about 200 and about 1,000 bp, about 200 and about 900 bp, about 200 and about 800 bp, about 200 and about 700 bp, about 200 and about 700 bp, about 200 and about 600 bp, about 200 and about 500 bp, about 200 and about 400 bp, about 200 and about 300 bp, about 500 and about 5,000 bp, about 500 and about 2,000 bp, about 500 and about 1,000 bp, about 500 and about 900 bp, about 500 and about 800 bp, about 500 and about 700 bp, about 500 and about 700 bp, or about 500 and about 600 bp.

In any of the preceding artificial nucleic acids, a TBS may be at least partially single-stranded or double-stranded. A transposase protein typically binds to a double-stranded TBS.

The invention also provides molecular constructs that include an artificial nucleic acid that includes a first end having a first TBS, a second end having a second TBS, and a linking segment disposed between the first TBS and the second TBS, such as any of those described above, in which the molecular construct includes a first transposase bound to the first TBS and a second transposase bound to the second TBS. In some instances, the linking segment prevents the first transposase and the second transposase from oligomerizing with each other. In some instances, the first transposase and the second transposase do not oligomerize with each other, for example, because they are of different types (e.g., the first transposase may be Tn5 or a biologically active variant thereof, and the second transposase may be Mu or a biologically active variant thereof). In some instances, more than one transposase binds to the first TBS or the second TBS, which may be due to the binding mode of the transposase. For example, Mu functions as a tetramer, and accordingly two Mu transposase proteins may bind a TBS.

The invention also provides TSCs having at least three of the molecular constructs described above, wherein the constructs are concatenated by oligomerization of a transposase in a first construct with a transposase in a second construct, and wherein at least two synaptic complexes are present in the complex. A TSC may include, for example, between 2 and 1000 or more synaptic complexes. For example, a TSC may include 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, 200, 300, 400, 500, 600, 700, 800, 900, 1000, or more synaptic complexes.

In some instances, each artificial nucleic acid in a TSC includes an IST. The ISTs present in a TSC may be identical. In other instances, the TSC may include a plurality of different ISTs. For example, a TSC may include at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, 200, 300, 400, 500, 600, 700, 800, 900, 1000, or more different ISTs.

The invention provides artificial nucleic acids that include a first end that includes a TBS and a second end that includes a ligatable segment (e.g., a sticky end). Such artificial nucleic acids can be bound by a transposase that binds to the TBS.

The invention further provides ligatable synaptic complexes that include a first transposase; a second transposase; a first artificial nucleic acid including a first end including a TBS and a second end, wherein the second end is ligatable (e.g., a sticky end); and a second artificial nucleic acid including a first end including a TBS and a second end, wherein the second end is ligatable (e.g., a sticky end), wherein the first transposase is bound to the TBS of the first artificial nucleic acid, the second transposase is bound to the TBS of the second artificial nucleic acid, and the first transposase and the second transposase are oligomerized. The sticky end may include a 5′ overhang or a 3′ overhang.

The invention further provides artificial nucleic acids that include a first end that includes a TBS and being linked to an affinity binding pair or a conjugating moiety (e.g., 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a sulfhydryl, a thiol, or a thioisocyanate group). The affinity binding pair or conjugating moiety can be positioned, for example, at the second end of the artificial nucleic acid. It is to be understood that the affinity binding pair or the conjugating moiety can be positioned at any suitable site on the artificial nucleic acid.

The invention also provides synaptic complexes that include a first transposase; a second transposase; a first artificial nucleic acid including a first end including a TBS and being linked to a component of a first affinity binding pair or a first conjugating moiety; and a second artificial nucleic acid including a first end including a TBS and being linked to a component of a second affinity binding pair or a second conjugating moiety, wherein the first transposase is bound to the TBS of the first artificial nucleic acid, the second transposase is bound to the TBS of the second artificial nucleic acid, and the first transposase and the second transposase are oligomerized. Any of the transposases described herein may be used in the compositions of the invention, including those described further below. For example, the transposase may be Tn3, Tn5, Tn9, Tn10, gamma-delta, Mu, piggyBac, Minos, Tc1, or Sleeping Beauty transposase or a biologically active variant thereof. The biologically active variant may be a hyperactive variant. Other transposases are known in the art and may also be used in the invention. Likewise, any of the TBSs described herein may be used in the compositions of the invention. In some instances, a transposase may be operably linked to a targeting moiety. The targeting moiety may be any targeting moiety described herein or known in the art. For example, the targeting moiety may be a polypeptide comprising a DNA binding domain (DBD) or an RNA-guided endonuclease. The DBD may be a zinc finger domain or a transcription activator-like (TAL) effector. The RNA-guided effector may be Cas9, Cpf1, C2c2, or a biologically active variant thereof (e.g., a nuclease-deficient variant).

### Transposases and Transposase Binding Sites

The compositions of the invention may include transposase(s) and transposase binding sites (TBSs) from any suitable transposition system known in the art. The transposition system may be from a virus (e.g., a phage or a retrovirus), a prokaryote (e.g., a bacterium), or a eukaryote (e.g., a fungus (e.g., yeast) or a mammal). In some instances, exemplary transposases that may be used include, but are not limited to, transposases from the transposon systems Tn1, Tn2, Tn3, Tn5, Tn7, Tn9, Tn10, Tn903, Tn1000/Gamma-delta, Minos, Sleeping beauty, piggyBac, Tol2, Mos1, Himar1, Hermes, Tol2, Minos, P-element, Tc1/mariner, Tc3, or biologically active variants thereof. For example, in some instances, Tn3, Tn5, Tn9, Tn10, gamma-delta, or Mu transposase or a biologically active variant thereof may be used. In some instances, the biologically active variant of a transposase, which may be naturally occurring or engineered, may include one or more modifications relative to a reference transposase (e.g., one or more (e.g., 1, 2, 3, 4, 5, 6, 7, 8, 9, 10, 11, 12, 13, 14, 15, 20, or more) amino acid substitutions, insertions, and/or deletions), which may affect the activity (e.g., transposition activity), binding (e.g., binding specificity or affinity), or other properties of the transposase. In particular embodiments, the biologically active variant may be a hyperactive variant, which may have increased transposition activity in vitro or in vivo.

Any suitable TBS may be used in the compositions of the invention. The TBS may be a TBS from the transposition systems Tn1, Tn2, Tn3, Tn5, Tn7, Tn9, Tn10, Tn903, Tn1000/Gamma-delta, Minos, Sleeping beauty, piggyBac, Tol2, Mos1, Himar1, Hermes, Tol2, Minos, P-element, Tc1/mariner, Tc3, or biologically active variants thereof. For example, in some instances, a TBS from the transposition systems Tn3, Tn5, Tn9, Tn10, gamma-delta, or Mu or a biologically active variant thereof may be used.

A TBS may be a naturally occurring TBS or a biologically active variant thereof. A biologically active variant may be naturally occurring or engineered, and may include insertions, deletions, and/or substitutions relative to a reference TBS. The biologically active variant TBS may affect the activity (e.g., transposition activity), binding (e.g., binding specificity or affinity), or other properties of the transposase(s) that bind to the TBS. The TBS may also include all or a minimal subset of a naturally occurring TBS. For example, the Tn7 transposon has 4 overlapping TnsB transposase binding sites on the right terminus and 3 widely spaced TnsB binding sites on the left terminus, but transposition can occur with a minimal subset of two TnsB binding sites on the right terminus (Parks et al., Plasmid 61(1):1-14, 2009). In some instances, the TBS may be or include a sequence that does not exist in nature (see, e.g., Goldhaber-Gordon et al., J. Biol. Chem. 277(10): 7703-7712, 2002), but still permits transposition by a transposase.

Many naturally occurring TBSs include inverted repeat nucleotide sequences at the termini of the transposable DNA fragment. These terminal inverted repeats are found in certain transposition systems, including those derived from Tn1, Tn2, Tn3, Tn5, Tn9, Tn10, and Tn903. In some instances, a TBS used in the invention may include terminal inverted repeats. In other instances, the TBS may lack inverted repeats, such as TBSs derived from the bacteriophage transposon Mu or the bacterial transposon Tn7.

Exemplary transposases and TBSs are described further below.

Tn5

Tn5 is a well-studied transposition system derived from E. coli which can be used in the context of the invention (see, e.g., Reznikoff, Mol. Microbiol. 47(5):1199-206, 2003). NCBI Accession No. U00004 provides the nucleic acid sequence of the E. coli Tn5 transposon. Tn5 encodes the transposase TnpA (UniProt Accession No. Q46731), which is also referred to herein as Tn5 transposase. The amino acid sequence of wild-type Tn5 transposase is shown below:

(SEQ ID NO: 25)
MITSALHRAADWAKSVFSSAALGDPRRTARLVNVAAQLAKYSGKSITIS
SEGSEAMQEGAYRFIRNPNVSAEAIRKAGAMQTVKLAQEFPELLAIEDT
TSLSYRHQVAEELGKLGSIQDKSRGWWVHSVLLLEATTFRTVGLLHQEW
WMRPDDPADADEKESGKWLAAAATSRLRMGSMMSNVIAVCDREADIHAY
LQDKLAHNERFVVRSKHPRKDVESGLYLYDHLKNQPELGGYQISIPQKG
VVDKRGKRKNRPARKASLSLRSGRITLKQGNITLNAVLAEEINPPKGET
PLKWLLLTSEPVESLAQALRVIDIYTHRWRIEEFHKAWKTGAGAERQRM
EEPDNLERMVSILSFVAVRLLQLRESFTLPQALRAQGLLKEAEHVESQS
AETVLTPDECQLLGYLDKGKRKRKEKAGSLQWAYMAIARLGGFMDSKRT
GIASWGALWEGWEALQSKLDGFLAAKDLMAQGIKI.

Biologically active variants of Tn5 transposase, including variants with amino acid substitutions, insertions, and/or deletions, may be used in the compositions of the invention. Biologically active Tn5 transposase variants with amino acid substitutions are known in the art. In some instances, a biologically active variant has an enhanced transposition rate relative to wild-type Tn5, and is thus considered hyperactive (see, e.g., U.S. Pat. Nos. 5,965,443; 5,925,545; and 6,159,736). For example, substitution of a lysine residue at amino acid 54 in place of the glutamic acid found in wild-type Tn5 transposase (E54K) has been shown to improve the avidity of the modified transposase for OE termini and to increase the transposition rate approximately 10-fold. Other mutations that have been associated with Tn5 transposase hyperactivity include a substitution of amino acid 372 (leucine) with proline (L372P) and a substitution of amino acid 56 (methionine) with alanine (M56A). The substitution mutations may be relative to the exemplary wild-type sequence of Tn5 transposase shown in SEQ ID NO: 25. A biologically active variant may include any combination of the preceding substitution mutations. For example, in some instances, the Tn5 transposase includes the substitution mutations E54K, M56A, and L372P. In other instances, the Tn5 transposase includes the substitution mutations E54K and L372P. Hyperactive Tn5 transposase proteins are commercially available, for example, Ez-Tn5™ transposase and Ez-Tn5™ Custom Transposome Construction Kits (Epicentre).

It is generally understood that to carry out transposition, Tn5 transposases bind a pair of inverted repeat nucleotide sequences that flank each side of the transposable DNA element. The inverted repeat sequences of the Tn5 transposase binding sites are referred to as the outside end (OE) (CTGACTCTTATACACAAGT (SEQ ID NO:1)) and inside end (IE) (CTGTCTCTTGATCAGATCT (SEQ ID NO:2)) (see, e.g., U.S. Pat. No. 5,965,443). Biologically active variants of a Tn5 TBS may be used, including end sequence variants that are associated with higher rates of transposition, for example, the hyperactive hybrid of the outside and inside ends (also referred to as “mosaic end” (ME)) CTGTCTCTTATACACATCT (SEQ ID NO:3), which differs from the wild-type OE sequence at positions 4, 17, and 18, as well as CTGTCTCTTATACAGATCT (SEQ ID NO:4), which differs from the wild-type OE sequence at positions 4, 15, 17, and 18 (see, e.g., U.S. Pat. No. 5,925,545). In some instances, a nucleic acid of the invention may include one or more Tn5 TBSs having a nucleic acid sequence selected from SEQ ID NOs:1-4 and/or a biologically active variant thereof.

Although the Tn5 inverted repeat sequences are often referred to as terminal repeat sequences, they need not be at the terminal ends of the donor DNA and need only to flank each side of the donor DNA to enable transposition (see, e.g., Johnson, et al., Nature 304:280, 1983 and U.S. Pat. No. 5,965,443). In some instances, the TBSs used in a nucleic acid of the invention may be any combination of two inverted sequences recognized by the Tn5 transposase, including the OE sequence, IE sequence and/or any other sequence variant (e.g., the ME sequence).

Mu

Another exemplary transposition system that can be harnessed by the present invention is from the Mu bacteriophage (see, e.g., Harshey, Microbiol. Spectr. 2(5), 2014). The complete nucleic acid sequence of the Mu genome is provided in NCBI Accession No. AF083977.1. Mu encodes the transposase MuA (UniProt Accession No. P07636), which is also referred to herein as Mu transposase. The amino acid sequence of wild-type Mu transposase is shown below:

(SEQ ID NO: 26)
MELWVSPKECANLPGLPKTSAGVIYVAKKQGWQNRTRAGVKGGKAIEYNA
NSLPVEAKAALLLRQGEIETSLGYFEIARPTLEAHDYDREALWSKWDNAS
DSQRRLAEKWLPAVQAADEMLNQGISTKTAFATVAGHYQVSASTLRDKYY
QVQKFAKPDWAAALVDGRGASRRNVHKSEFDEDAWQFLIADYLRPEKPAF
RKCYERLELAAREHGWSIPSRATAFRRIQQLDEAMVVACREGEHALMHLI
PAQQRTVEHLDAMQWINGDGYLHNVFVRWFNGDVIRPKTWFWQDVKTRKI
LGWRCDVSENIDSIRLSFMDVVTRYGIPEDFHITIDNTRGAANKWLTGGA
PNRYRFKVKEDDPKGLFLLMGAKMHWTSVVAGKGWGQAKPVERAFGVGGL
EEYVDKHPALAGAYTGPNPQAKPDNYGDRAVDAELFLKTLAEGVAMFNAR
TGRETEMCGGKLSFDDVFEREYARTIVRKPTEEQKRMLLLPAEAVNVSRK
GEFTLKVGGSLKGAKNVYYNMALMNAGVKKVVVRFDPQQLHSTVYCYTLD
GRFICEAECLAPVAFNDAAAGREYRRRQKQLKSATKAAIKAQKQMDALEV
AELLPQIAEPAAPESRIVGIFRPSGNTERVKNQERDDEYETERDEYLNHS
LDILEQNRRKKAI.

Biologically active variants of Mu transposase, including variants with deletions, insertions, or amino acid substitutions, are known in the art and can be used in the invention. For example, truncated Mu transposase variants, such as the truncation mutant Mu(77-663), which contains amino acids 77-663 of wild-type Mu transposase, have been described as hyperactive (see Goldhaber-Gordon et al., J. Biol. Chem. 277(10):7694-702, 2002). Hyperactive Mu variants with amino acid substitution mutations are also known in the art (see, e.g., U.S. Pat. No. 9,234,190). For example, a hyperactive Mu transposase variant may include one or more (e.g., 1, 2, 3, 4, 5, 6, 7, 8, 9, 10, 11, 12, 13, 14, 15, 16, 17, 18, 19, 20, 21, 22, 23, 24, 25, 26, or 26) amino acid substitution mutations selected from the group consisting of A59V, D97G, W160R, E179V, E233K, E233V, Q254R, E258G, G302D, 1335T, G340S, W345C, W345R, M374V, F447S, F464Y, R478H, R478C, E482K, E483G, E483V, M4871, V495A, V507A, Q539H, Q539R, and I617T. The mutations may be relative to the exemplary wild-type sequence of Mu transposase shown in SEQ ID NO:26. For example, the substitution mutation may be E223V. In other instances, the Mu variant may include the substitution mutations W160R, E233K, and W345R.

Each end of the Mu transposon includes three Mu binding sites: L1, L2, and L3 on the left end and R1, R2, and R3 on the right end. The nucleic acids of these Mu TBSs are as follows: L1 (TGTATTGATTCACTGAAGTACGAAAA (SEQ ID NO:5)), L2 (CCTTAATCAATGAAACGCGAAAG, SEQ ID NO:6), L3 (TTGTTTCATTGAAAATACGAAAA, SEQ ID NO:7), R1 (TGAAGCGGCGCACGAAAAATGCGAAAA, SEQ ID NO:8), R2 (GCGTTTCACGATAAATGCGAAAA, SEQ ID NO:9), and R3 (CCGTTTCATTTGAAGCGCGAAAA, SEQ ID NO:10). The Mu binding sites have a 22-nucleotide consensus sequence, YGTTTCAYNNRAARYRCGAAAR (SEQ ID NO:11), wherein Y denotes a pyrimidine (C or T), R denotes a purine (G or A), and N denotes any nucleotide. In some embodiments, a nucleic acid of the invention may include one or more Mu TBSs that include a nucleic acid sequence selected from SEQ ID NO:5, SEQ ID NO: 6, SEQ ID NO:7, SEQ ID NO:8, SEQ ID NO:9, SEQ ID NO:10, SEQ ID NO:11, and/or a biologically active variant thereof.

Previous studies indicate that under certain conditions, only a minimal number of elements are required for Mu transposition, namely a short Mu right-end donor DNA that includes the R1 (SEQ ID NO. 8) and R2 (SEQ ID NO. 9) binding sites, the Mu transposase, and a linear target DNA (see, e.g., Savilahti, EMBO J. 14(19):4893-4903, 1995). Therefore, in some instances, a nucleic acid of the invention may include a TBS that includes the nucleic acid sequences of SEQ ID NO:8 and SEQ ID NO:9. Alternatively, the Mu TBS may include a sequence that does not occur in nature, but nonetheless permits transposition by the Mu transposase. For example, FIG. 2 of Goldhaber-Gordon et al. J. Biol. Chem. 277(10): 7703-7712, 2002 shows the nucleic acid sequences of 18 non-Mu sequences that function analogously to Mu TBSs.

Tn10

The transposase and TBSs of the Tn10 transposition system, or biologically active variants thereof, may be used in the context of the invention. NCBI Accession No. AY319289.1 provides the nucleic acid sequence of the E. coli Tn10 transposon. Tn10 encodes the transposase TnpA (UniProt Accession No. Q70BL4), also referred to herein as Tn10 transposase. The amino acid sequence of wild-type Tn10 transposase is shown below:

(SEQ ID NO: 27)
MCELDILHDSLYQFCPELHLKRLNSLTLACHALLDCKTLTLTELGRNLPT
KARTKHNIKRIDRLLGNRHLHKERLAVYRWHASFICSGNTMPIVLVDWSD
IREQKRLMVLRASVALHGRSVTLYEKAFPLSEQCSKKAHDQFLADLASIL
PSNTTPLIVSDAGFKVPWYKSVEKLGWYWLSRVRGKVQYADLGAENWKPI
SNLHDMSSSHSKTLGYKRLTKSNPISCQILLYKSRSKGRKNQRSTRTHCH
HPSPKIYSASAKEPWVLATNLPVEIRTPKQLVNIYSKRMQIEETFRDLKS
PAYGLGLRHSRTSSSERFDIMLLIALMLQLTCWLAGVHAQKQGWDKHFQA
NTVRNRNVLSTVRLGMEVLRHSGYTITREDLLVAATLLAQNLFTHGYALG
KL.

Hyperactive Tn10 transposase variants have been described (see, e.g., Way, Gene 32(3):369-79, 1984) and may be used in the invention.

Like Tn5 transposase, the Tn10 transposase typically binds a pair of inverted repeat nucleotide sequences that flank each side of the transposable DNA element. A Tn10 TBS may include Tn10 inverted repeat sequences, generally referred to as the outside ends (OE) and inside ends (IE), which have a consensus sequence of CTGAKRRATCCCCTMATRATTTY (SEQ ID NO:12), wherein Y denotes a pyrimidine (C or T), R denotes a purine (G or A), M denotes A or G, and K denotes G or T (Mizuuchi, Annu. Rev. Biochem. 61:1011-51, 1992). In some instances, a nucleic acid of the invention may include one or more Tn10 TBSs having the nucleic acid sequence of SEQ ID NO:12 and/or a biologically active variant thereof.

Tn7

The Tn7 transposition system is another transposition system known in the art (see, e.g., Parks et al., Plasmid. 61(1):1-14, 2009). The Tn7 transposon encodes the transposases TnsA (Uniprot Accession No. P13988; also referred to as TnpA) and TnsB (Uniprot Accession No. P13989; also referred to as TnpB).

The amino acid sequence of wild-type Tn7 TnsA is shown below:

(SEQ ID NO: 28)
MAKANSSFSEVQIARRIKEGRGQGHGKDYIPWLTVQEVPSSGRSHRIYSH
KTGRVHHLLSDLELAVFLSLEWESSVLDIREQFPLLPSDTRQIAIDSGIK
HPVIRGVDQVMSTDFLVDCKDGPFEQFAIQVKPAAALQDERTLEKLELER
RYWQQKQIPWFIFTDKEINPVVKENIEWLYSVKTEEVSAELLAQLSPLAH
ILQEKGDENIINVCKQVDIAYDLELGKTLSEIRALTANGFIKFNIYKSFR
ANKCADLCISQVVNMEELRYVAN.

The amino acid sequence of wild-type Tn7 TnsB is shown below:

(SEQ ID NO: 29)
MWQINEVVLFDNDPYRILAIEDGQVVWMQISADKGVPQARAELLLMQYLD
EGRLVRTDDPYVHLDLEEPSVDSVSFQKREEDYRKILPIINSKDRFDPKV
RSELVEHVVQEHKVTKATVYKLLRRYWQRGQTPNALIPDYKNSGAPGERR
SATGTAKIGRAREYGKGEGTKVTPEIERLFRLTIEKHLLNQKGTKTTVAY
RRFVDLFAQYFPRIPQEDYPTLRQFRYFYDREYPKAQRLKSRVKAGVYKK
DVRPLSSTATSQALGPGSRYEIDATIADIYLVDHHDRQKIIGRPTLYIVI
DVFSRMITGFYIGFENPSYVVAMQAFVNACSDKTAICAQHDIEISSSDWP
CVGLPDVLLADRGELMSHQVEALVSSFNVRVESAPPRRGDAKGIVESTFR
TLQAEFKSFAPGIVEGSRIKSHGETDYRLDASLSVFEFTQIILRTILFRN
NHLVMDKYDRDADFPTDLPSIPVQLWQWGMQHRTGSLRAVEQEQLRVALL
PRRKVSISSFGVNLWGLYYSGSEILREGWLQRSTDIARPQHLEAAYDPVL
VDTIYLFPQVGSRVFWRCNLTERSRQFKGLSFWEVWDIQAQEKHNKANAK
QDELTKRRELEAFIQQTIQKANKLTPSTTEPKSTRIKQIKTNKKEAVTSE
RKKRAEHLKPSSSGDEAKVIPFNAVEADDQEDYSLPTYVPELFQDPPEKD
ES.

TnsA and TnsB are thought to form a heteromeric transposase. TnsB is a DDE-type transposase that catalyzes concerted breakage and rejoining reactions, joining the 3′-hydroxyl of the donor ends to the 5′-phosphate groups at the insertion site of the target DNA. TnsA structurally resembles a restriction endonuclease, and carries out the nicking reaction on the opposite strand of the donor DNA molecule. Accessory protein TnsC is thought to modulate the activity of the heteromeric TnsAB transposase, and activates transposition when complexed with target DNA and a target selection protein, TnsD or TnsE. TnsC variants have been isolated that can promote transposition in the absence of TnsD or TnsE. In some instances, biologically active variants of TnsA, TnsB, TnsC, TnsD, and/or TnsE may be used in the context of the invention, including variants with deletions, insertions, or amino acid substitutions. Hyperactive Tn7 transposase variants have previously been described. For example, Table 1 of Lu et al., (EMBO J. 19(13):3446-57, 2000) describes several TnsA and TnsB substitution mutants, including TnsA S69N, E73K, A65V, E185K, Q261Z, G239S, G239D, E185K, and Q261Z, as well as TnsB M3661, A325T, and A325V. In some instances, a biologically active Tn7 variant may include one or more of any of the preceding substitution mutations.

Seven Tn7 transposase binding sites are located on each end of the transposon, including four overlapping TnsB binding sites on the right end and three widely spaced TnsB binding sites on the left end. The consensus sequence of the seven Tn7 transposase binding sites is TGAYAATAAAGTTGATTATACT (SEQ ID NO:13), wherein Y denotes a pyrimidine (C or T) (see, e.g., Parks et al., Plasmid. 61(1):1-14, 2009). In some instances, a nucleic acid of the invention may include one or more Tn7 TBSs that include the nucleic acid sequence of SEQ ID NO:13 and/or a biologically active variant thereof.

Tn3

The Tn3 transposon is another transposition system known in the art (see, e.g., Ichikawa et al., Proc. Natl. Acad. Sci. USA 84(23):8220-4, 1987). NCBI Accession No. V00613.1 provides the nucleic acid sequence of the E. coli Tn3 transposon. The Tn3 transposon encodes the transposase TnpA (UniProt Accession No. P03008), also referred to herein as Tn3 transposase, and the resolvase TnpR (Uniprot Accession No. POADI2). Tn3 utilizes a replicative transposition mechanism, with a first stage of replicative integration catalyzed by the Tn3 transposase that results in a “cointegrate” DNA molecule containing two copies of the transposon, followed by a resolution stage catalyzed by the resolvase that separates the donor and target DNA molecules.

The Tn3 transposase binds to terminal inverted repeat sequences comprising a left terminal inverted repeat, GGGGTCTGACGCTCAGTGGAACGAAAACTCACGTTAAG (SEQ ID NO: 14), and a right terminal inverted repeat, CTTAACGTGAGTTTTCGTTCCACTGAGCGTCAGACCCC (SEQ ID NO: 15). In some instances, a nucleic acid of the invention may include one or more Tn3 TBSs that includes the nucleic acid sequence of SEQ ID NO: 14, SEQ ID NO:15, and/or a biologically active variant thereof

Gamma-Delta

Some embodiments of the present invention may use the transposase and TBSs from the gamma-delta transposon, also referred to as Tn1000 (see, e.g., Broom, DNA Seq. 5(3):185-9, 1995). Gamma-delta is related to Tn3. NCBI Accession No. D16449.1 provides the nucleic acid sequence of the E. coli gamma delta transposon. The gamma delta transposon encodes the transposase TnpA (UniProt Accession No. Q00037), also referred to herein as gamma-delta transposase, and a resolvase TnpR (UniProt Accession No. P03012).

The gamma-delta transposase binds to terminal inverted repeat sequences that include a “delta end” terminal inverted repeat, GGGGTTTGAGGGCCAATGGAACGAAAACGTACGTTAAG (SEQ ID NO:16), and a “gamma end” terminal inverted repeat, ATAAACGTACGTTTTCGTTCCATTGGCCCTCAAACCCC (SEQ ID NO:17). See, e.g., Maekawa et al., Jpn. J. Genet. 69(3):269-85, 1994. In some instances, a nucleic acid of the invention may include one or more gamma-delta TBSs that include the nucleic acid sequence of SEQ ID NO:16, SEQ ID NO:17, and/or a biologically active variant thereof.

piggyBac™

The piggyBac™ (pB) transposase, TBSs, and biologically active variants thereof may be used in some embodiments of the invention (see, e.g., Yusa, Microbiol. Spectr. 3(2), 2015). The pB transposon was isolated from the cabbage looper moth Trichoplusia ni genome. A number of pB-like transposons have also been identified in a variety of species. NCBI Accession No. J04364.2 provides the nucleic acid sequence of the T. ni pB transposon, which encodes the pB transposase (UniProt Accession No. Q27026). pB transposase typically integrates at TTAA sites in a target DNA. Biologically active variants of the pB transposase, including variants with deletions, insertions, or amino acid substitutions, may be used in the invention. Hyperactive pB variants with amino acid substitutions have previously been described (see, e.g., Yusa et al., Proc. Natl. Acad. Sci. USA 108(4):1531-6, 2011 and U.S. Pat. No. 8,399,643). pB transposon systems are commercially available (Transposagen).

pB TBSs are known in the art (see, e.g., Cary et al. Virology 172(1):156-169, 1989). The pB transposon includes 13-bp terminal inverted repeats and has additional inverted repeats of 19 bp in length located asymmetrically with respect to the element.

Minos

Minos transposase, TBSs, and biologically active variants thereof can be used in some embodiments of the invention. The Minos transposon was identified in the genome of the fruit fly Drosophila hydei(see, e.g., Pavlopoulos et al., Genome Biol. 8 (Suppl 1), 2007). NCBI Accession No. X61695.1 provides the nucleic acid sequence of the Minos transposon, which encodes the Minos transposase (Uniprot Accession No. Q9U986).

The Minos transposase binds to a 5′ inverted terminal repeat (ITR) that includes the following sequence:

(SEQ ID NO: 23)
CGCTTAACTTAATACGAGCCCCAACCACTATTAATTCGAACAGCATGTT
TTTTTTGCAGTGCGCAATGTTTAACACACTATATTATCAATACTACTAA
AGATAACACATACCAATGCATTTCGTCTCAAAGAGAATTTTATTCTCTT
CACGACGAAAAAAAAAGTTTTGCTCTATTTCCAACAACAACAAAAATAT
GAGTAATTTATTCAAACGGTTTGCTTAAGAGATAAGAAAAAAGTGACCA
CTATTAATTC

and a 3′ ITR having the following sequence:

(SEQ. ID NO: 24)
ATAGTAAATCACATTACGCCGCGTTCGAATTAATAGTGGTCACTTTTTTC
TTATCTCTTAAGCAAACCGTTTGAATAAATTACTCATATTTTTGTTGTTG
TTGGAAATAGAGCAAAACTTTTTTTTTCGTCGTGAAGAGAATAAAATTCT
CTTTGAGACGAAATGCATTGGTATGTGTTATCTTTAGTAGTATTGATAAT
ATAGTGTGTTAAACATTGCGCACTGCAAAAAAAACATGCTGTTCGAATTA
ATAGT.

In some embodiments, a nucleic acid of the invention may include one or more Minos TBSs selected from SEQ ID NO:23, SEQ ID NO:24, and/or a biologically active variant thereof.

Sleeping Beauty

Sleeping Beauty (SB) transposase, TBSs, and biologically active variants thereof may be used in some embodiments of the invention. SB is a synthetic transposase Tc1/mariner-type transposase that was re-constructed from the genomes of salmonid fish (Ivics et al. Cell 91(4):501-510, 1997). SB transposases are known in the art (see, e.g., International Patent Application Publication No. WO1999/025817 and U.S. Pat. No. 6,613,752). The amino acid sequence of a reference SB transposase is shown below:

(SEQ ID NO: 30)
MGKSKEISQDLRKKIVDLHKSGSSLGAISKRLKVPRSSVQTIVRKYKHHG
TTQPSYRSGRRRVLSPRDERTLVRKVQINPRTTAKDLVKMLEETGTKVSI
STVKRVLYRHNLKGRSARKKPLLQNRHKKARLRFATAHGDKDRTFWRNVL
WSDETKIELFGHNDHRYVWRKKGEACKPKNTIPTVKHGGGSIMLWCGFAA
GGTGALHKIDGIMRKENYVDILKQHLKTSVRKLKLGRKWVFQMDNDPKHT
SKVVAKWLKDNKVKVLEWPSQSPDLNPIENLWAELKKRVRARRPTNLTQL
HQLCQEEWAKIHPTYCGKLVEGYPKRLTQVKQFKGNATKY.

Hyperactive SB variants that include amino acid substitutions are known in the art (see, e.g., U.S. Pat. Nos. 7,985,739 and 9,228,180). For example, a hyperactive SB variant may include one or more substitution mutations selected from the following: K13A, K14R, K13D, K30R, K33A, T83A, I100L, R115H, R143L, R147E, A205K/H207V/K208R/D210E; H207V/K208R/D210E; R214D/K215A/E216V/N217Q; M243H; M243Q; E267D; T314N; and G317E (see, e.g., U.S. Pat. No. 9,228,180). In some instances, the hyperactive SB variant may include a K14R substitution mutation. The substitution mutations may be relative to the reference sequence of SB transposase shown in SEQ ID NO:30.

SB TBSs are also known in the art (see, e.g., International Patent Application Publication No. WO 1998/040510 and U.S. Pat. No. 6,613,752). These TBSs and/or biologically active variants thereof may be used in the nucleic acids of the invention.

### Targeting Moieties

To promote transposition to specific regions of a target nucleic acid (e.g., DNA), a transposase present in a composition of the invention (e.g., a TSC) may be targeted to particular nucleotide sequences using a targeting moiety, which can result in biased or targeted transposition of transposable nucleic acids present in a TSC. Any suitable targeting moiety known in the art or described herein may be used, so long as it can be operably linked to the transposase. The targeting moiety may be a fusion partner in a fusion protein that includes a transposase. For example, a fusion protein can include a transposase and a targeting moiety and may optionally include an intervening linker. The targeting moiety may be located N-terminally or C-terminally relative to the transposase. In other examples, the targeting moiety may be covalently or non-covalently conjugated to the transposase. The targeting moiety may be naturally occurring or engineered.

The targeting moiety may be a polypeptide that includes a DNA binding domain (DBD) that confers binding preference or specificity to a defined nucleotide sequence. For example, DBDs may include zinc finger motifs, which are well-known in the art, including but not limited to the zinc finger DBDs Sp1, ZNF202, Gal4, E2C, Zif268, and TetR. The zinc finger motif may be derived, for example, from a Cys2-His2 type zinc finger. Fusion proteins that include transposases and zinc finger motifs are known in the art. For example, fusion proteins that include Sleeping Beauty (SB) transposase and a zinc finger DBD have been constructed using the DBD of Sp1, ZNF202, E2C, Gal4, or TetR (see, e.g., Wilson et al., FEBS Letters 579:6205-9, 2005, Ivics et al., Mol. Ther. 5(6):1137-44, 2007; and Yant et al., Nucleic Acids Res. 35(7):e50, 2007). The piggyBac and Mos1 transposases have each been fused to the DBD of Gal4 (see, e.g., Maragathavally et al., FASEB J. 20(11):1880-2, 2006 and Wu et al., Proc. Natl. Acad. Sci. USA 103(41):15008-13, 2006). In another example, the ISY100 transposase has been fused to the DBD of Zif268 (see, e.g., Feng et al., Nucleic Acids Res. 38(4):1204-1216).

Zinc finger motifs can be engineered to bind to a desired DNA sequence. A known “recognition code” that relates the amino acids of a single zinc finger motif to its associated DNA target can be utilized as a guide for the design of zinc finger motif DBDs that bind to particular DNA sequences, for example, using modular assembly (see, e.g., Bhakta et al., Methods Mol. Biol. 649:3-30, 2010). Alternatively, selection-based approaches (e.g., phage display or bacterial two-hybrid systems) can be used to obtain zinc finger motifs that bind to particular DNA sequences (see, e.g., Maeder et al., Mol. Cell. 31:294-301, 2008). A DBD may include, for example, 2, 3, 4, 5, 6, 7, 8, 9, 10, or more zinc finger motifs.

Other DBDs that may be used include DBDs belonging to transcriptional regulators (see, e.g., Szabó et al., FEBS Letters 550(1-3):46-50, 2003 and Imre et al., FEMS Microbiology Letters 317(1):52-9, 2011) and transcription activator-like effectors (TAL effectors), which are type III effector proteins that are secreted by Xanthomonas species and can bind to promoter sequences in the host plant. Like zinc finger motifs, TAL effectors can be engineered to bind to specific DNA sequences (see, e.g., Boch et al., Science 326(5959):1509-1512, 2009). Other types of DBDs are known in the art and can be used as targeting moieties, including, for example, helix-turn-helix motifs, leucine zipper domains, winged helix domains, winged helix turn helix domains, helix-loop-helix domains, and HMG box domains.

In some embodiments, the targeting moiety may include an RNA- or DNA-guided endonuclease, including but not limited to Cas9, Cpf1, C2c2, and Argonaute. In preferred embodiments, the RNA- or DNA-guided endonuclease is nuclease-deficient or nuclease-null. For example, the transposase may be fused to a RNA- or DNA-guided endonuclease in a fusion protein.

The Cas9 protein (CRISPR-associated protein 9), which is derived from type II CRISPR (clustered regularly interspaced short palindromic repeats) systems, is an RNA-guided DNA endonuclease that can be programmed to target new sites by modifying its guide RNA sequence (see, e.g., Wang et al., Annu Rev Biochem 85:227-64, 2016; and U.S. Pat. No. 8,795,965). In some instances, a nuclease-deficient or nuclease-null Cas9 (e.g., dCas9, which includes point mutations in two catalytic residues (D10A and H840A) of Cas9) may be utilized in the context of the invention as a targeting moiety that can be utilized in vitro. Previous work has established that Cas9 fusion proteins can be utilized for a variety of applications, including transcriptional activation, targetable DNA methylation, and enhanced specificity of DNA cleavage (see, e.g., Mali et al., Nat Biotechnol. 31(9):833-8, 2013; Vojta et al., Nucleic Acids Res. 44(12):5615-28, 2016; Guilinger et al., Nat. Biotechnol. 32(6):577-82, 2014; U.S. Pat. No. 9,388,430; and U.S. Patent Application Publication Nos. 2015/0291965 and 2016/0177304).

Cpf1 or C2c2 can also be used instead of Cas9 in the context of the invention. Cpf1 is distinct from Cas9 in that it is a single RNA-guided endonuclease lacking trans-activating crRNA (tracrRNA), but with comparable targeting specificity to Cas9 (see, e.g., Zetsche et al., Cell 163(3):759-71, 2015; Kleinstiver et al., Nat. Biotechnol. 34(8):869-74, 2016; Kim et al., Nat. Biotechnol. 34(8):863-8, 2016; and U.S. Patent Application Publication No. 2016/0208243). C2c2 is a programmable RNA-guided RNA endonuclease that targets single-stranded RNA, with nuclease activity that, like Cas9 and Cpf1, can be made nuclease-deficient (see, e.g., Abudayyeh et al., Science 353(6299):aaf5573, 2016). In some instances, Argonaute can be utilized. Prokaryotic Argonaute variants have been described that act as DNA-guided DNA endonucleases, with inactivating mutations also described (see, e.g., Swarts et al., Nature 507(7491):258-61, 2014; Miyoshi et al., Nat. Commun. 7:11846, 2016; and Gao et al., Nat. Biotechnol. 34(7):768-73, 2016).

In some embodiments, the transposase may be targeted to defined nucleotide sequences by non-covalent binding to a polypeptide that includes a sequence-specific DBD. Some DNA-modifying enzymes naturally utilize such protein interactions for targeted transposition. For example, in the Ty5 retrotransposon system, the yeast Ty5 integrase is targeted to specific regions of genomic DNA by the DNA binding protein Sir4p. The specificity of Ty5 integration can be altered by fusing alternate DBDs to Sir4p (see, e.g., Zhu et al., Proc. Natl. Acad. Sci. USA 100(10):5891-5, 2003). In situations where a transposase does not naturally interact with a DNA binding partner, additional components or domains may be fused or conjugated to the transposase and/or DNA binding protein to promote protein-protein interactions. Further, the DBD of the interacting protein may be modified to confer the desired target sequence specificity.

In another embodiment, the targeting moiety may include a DNA or RNA or PNA oligonucleotide with a nucleotide sequence that is at least partially complementary to a sequence present in the target nucleic acid (e.g., DNA). Hybridization of the oligonucleotide to the target nucleic acid could target the transposase to the target sequence. An oligonucleotide targeting moiety may be covalently or non-covalently conjugated to the transposase, for example, by modifying both the oligonucleotide and transposase with complementary coupling moieties. Oligonucleotides and proteins can be conjugated using a variety of coupling approaches, including any of the approaches outlined in Mao et al., Chem. Soc. Rev. 40:5730-44, 2011. For example, methods of covalent conjugation may include site-specific coupling of thiol-modified oligonucleotides by disulfide bond formation to a transposase engineered with either an accessible cysteine residue (see, e.g., Corey et al., J. Am. Chem. Soc. 111(22):8523-5, 1989) or an alpha-thioester (see, e.g., Takeda et al., Bioorg. Med. Chem. Lett. 14(10):2407-10, 2004). Examples of non-covalent oligo-protein conjugation methods include, but are not limited to, streptavidin-biotin, Ni-NTA-hexahistidine, and antibody-hapten based coupling methods.

### Affinity Binding Pairs

The compositions of the invention may include affinity binding pairs. Affinity binding pairs may be used to link two or more moieties non-covalently. For example, a linking segment may include one or more affinity binding pairs that link the linking segment to two nucleic acids containing TBSs. Any suitable affinity binding pair known in the art or described herein may be used. Exemplary, non-limiting affinity binding pairs include biotin-biotin binding protein (e.g., biotin-streptavidin, biotin-avidin, and biotin-NeutrAvidin™), ligand-receptor, antigen-antibody or antigen binding fragment, hapten-anti-hapten, and Ig binding protein-lg. Components of affinity binding pairs can be conjugated to compositions of the invention (e.g., artificial nucleic acids (e.g., artificial nucleic acids containing TBSs)), linking segments (e.g., a nucleic acid, a protein, or a non-nucleotide chemical moieties (e.g., a polymer, e.g., a polyether such as polyethylene glycol (PEG))) using approaches described herein or others known in the art.

Biotin-biotin binding proteins are well-characterized affinity binding pairs. Biotin or biologically active variants and analogues thereof may be used. Avidin and other biotin binding proteins bind with considerable affinity to biotin. Exemplary biotin binding proteins include avidin, streptavidin, NeutrAvidin™ (a deglycosylated version of avidin), CaptAvidin™, and the like. The biotin binding protein may be, for example, tetrameric, dimeric, or monomeric. Biotin and biotin binding proteins can be conjugated using routine approaches to nucleic acids, proteins, or non-nucleotide chemical moieties (e.g., a polymer, e.g., a polyether such as polyethylene glycol (PEG)). For example, a variety of amine-reactive, sulfhydryl-reactive, carboxyl-reactive, carbohydrate/aldehyde-reactive, photo-reactive, and other biotinylation reagents are commercially available. Biotin binding proteins, including avidin, streptavidin, and NeutrAvidin™, are commercially available and can be conjugated using routine approaches to nucleic acids, proteins, or non-nucleotide chemical moieties (e.g., a polymer, e.g., a polyether such as polyethylene glycol (PEG)).

The binding pair may be a ligand-receptor binding pair. A wide variety of receptors and their corresponding ligands are known in the art. The binding pair may include a fragment of a receptor that binds to a ligand. The receptor can be, for example, a cytokine receptors (e.g., vascular endothelial growth factor (VEGF) receptors (e.g., VEGFR-1 and VEGFR-2), tumor necrosis factor (TNF) receptors (e.g., TNF receptor 2), and the like). Soluble receptors, including engineered soluble receptors that include extracellular binding portions of receptors fused to Fc regions, are known in the art (e.g., etanercept, a soluble TNF receptor 2 protein that binds to TNF, and aflibercept, a soluble VEGF receptor that binds to VEGF).

A wide variety of antibodies and the antigens to which they bind are known in the art, and any suitable antigen-antibody or antigen binding fragment thereof may be used in the invention. Exemplary antigen-antibody (or antigen binding fragment) binding pairs include digoxigenin/anti-digoxigenin; 2,4-dinitrophenyl (DNP)-triethylene glycol (TEG)/anti-DNP antibodies; fluorescein/anti-fluorescein antibodies; and the like.

A number of Ig binding proteins are known in the art and can be used in the invention, for example, protein A, protein G, protein L, protein M, binding immunoglobulin protein (BiP), and immunoglobulin-binding protein 1 (IGBP1), or biologically active variants thereof. An Ig binding protein may bind to the Fc region of an immunoglobulin, or a fragment thereof.

### Conjugation Approaches

Any suitable conjugation approach may be used to covalently link compositions of the invention.

For example, a linking segment may be conjugated to two nucleic acids containing TBSs. A variety of conjugation reactions are known in the art and can be used in the context of the invention, for example, a cycloaddition (e.g., an azide-alkyne Huisgen cycloaddition (e.g., a copper(I)-catalyzed azide-alkyne cycloaddition (CuAAC) or a strain-promoted azide-alkyne cycloaddition (SPAAC))), amide or thioamide bond formation, a pericyclic reaction, a Diels-Alder reaction, sulfonamide bond formation, alcohol or phenol alkylation, a condensation reaction, disulfide bond formation, or a nucleophilic substitution.

In some instances, a composition of the invention may include a conjugating moiety. A conjugating moiety includes at least one functional group that is capable of undergoing a conjugation reaction, for example, any conjugation reaction described in the preceding paragraph. The conjugation moiety can include, without limitation, a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a sulfhydryl, a thiol, or a thioisocyanate group.

### Tethered Synaptic Complexes and Methods of Making the Same

The invention provides TSCs as well as methods of making TSCs. In general, the methods involve contacting a nucleic acid of the invention that includes one or more TBSs with transposases that are able to bind one or more of the TBSs. The nucleic acids and transposases themselves may be prepared using any suitable method known in the art. FIGS. 2A, 2B, 3, 4A, and 4B above illustrate exemplary methods for making TSCs. These and other additional methods of making TSCs are described further below.

A transposase typically binds to a double-stranded TBS. An exemplary method of making a TSC involves providing a double-stranded nucleic acid molecule that includes a TBS at each terminus, contacting the double-stranded nucleic acid with transposases that bind to the TBSs for sufficient time and under suitable conditions to form a subunit that includes the nucleic acid and one or more transposases bound to each TBS, and subsequently forming TSCs by allowing the subunits to oligomerize or polymerize under suitable conditions.

An alternate method for making TSCs is as follows. A transposable nucleic acid can be made that includes a single-stranded TBS which is inactive for transposase binding. At a suitable time, the single-stranded TBS can be converted to a double-stranded TBS that is active for transposase binding. The single-stranded TBS can be converted to a double-stranded TBS by a number of approaches, including, for example, hybridizing a complementary nucleic acid to the single-stranded TBS; extending a nucleic acid with DNA polymerase to form a double-stranded TBS; or ligating a hybridized nucleic acid to form a double-stranded transposase binding site. The resulting double-stranded TBSs can be contacted with transposases, for example, as described above. The resulting subunits can be used to form TSCs by oligomerizing or polymerizing the subunits under suitable conditions. In one embodiment, a first nucleic acid with a binding site for transposase is annealed to a partially complementary second nucleic acid so that a double-stranded nucleic acid in which two TBSs are inverted relative to one another can be prepared in vitro and used in a method of making a TSC. The annealed nucleic acids can be extended by DNA polymerase to convert single-stranded portions of the nucleic acid to fully double-stranded nucleic acid, for example, as illustrated in FIG. 2A.

In some instances, TSC subunits may be prepared by linking two or more nucleic acids, at least two of which include a double-stranded TBS that is bound to a transposase. See, e.g., FIGS. 3, 4A, 4B, 4C, and 4D. The bound transposases may be identical or biologically active variants that are able to oligomerize, or they may be different types of transposases (e.g., Tn5 and Mu) that do not oligomerize.

Two or more nucleic acids can be linked using any suitable approach, including non-covalent linkage (e.g., via an affinity binding pair) or covalent linkage (e.g., via a conjugation reaction, including ligation reactions).

In some instances, two or more nucleic acids may be non-covalently linked using affinity binding pairs. For example, the method may include (a) providing at least a first synaptic complex and a second synaptic complex, where (i) the first synaptic complex includes a first pair of oligomerized transposases, wherein one member of the pair is bound to a first nucleic acid including a TBS and being linked to a first component of an affinity binding pair; and (ii) the second synaptic complex includes a second pair of oligomerized transposases, wherein one member of the pair is bound to a second nucleic acid including a TBS and being linked to a second component of the affinity binding pair or a second conjugating moiety; and (b) linking the first synaptic complex to the second synaptic complex, thereby preparing a TSC. The linking of (b) may include incubating the first synaptic complex and the second synaptic complex under conditions suitable for binding of the first component and the second component of the affinity binding pair.

In other instances, two or more nucleic acids may be covalently linked using a conjugation reaction. For example, the method may include (a) providing at least a first synaptic complex and a second synaptic complex, wherein (i) the first synaptic complex includes a first pair of oligomerized transposases, wherein one member of the pair is bound to a first nucleic acid comprising a TBS and being linked to a first conjugating moiety; and (ii) the second synaptic complex includes a second pair of oligomerized transposases, wherein one member of the pair is bound to a second nucleic acid including a TBS and being linked to second conjugating moiety; and (b) linking the first synaptic complex to the second synaptic complex, thereby preparing a TSC. The linking of (b) includes reacting the first conjugating moiety with the second conjugating moiety under conditions suitable to form a covalent bond. Any suitable conjugation reaction may be used, for example, a cycloaddition (e.g., an azide-alkyne Huisgen cycloaddition (e.g., a copper(I)-catalyzed azide-alkyne cycloaddition (CuAAC) or a strain-promoted azide-alkyne cycloaddition (SPAAC)), amide or thioamide bond formation, a pericyclic reaction, a Diels-Alder reaction, sulfonamide bond formation, alcohol or phenol alkylation, a condensation reaction, disulfide bond formation, or a nucleophilic substitution.

In some instances, two or more nucleic acids may be covalently linked by ligation. For example, the method may include: (a) providing at least a first synaptic complex and a second synaptic complex, wherein (i) the first synaptic complex includes a first pair of oligomerized transposases, wherein one member of the pair is bound to a first nucleic acid including a TBS and a ligatable end (e.g., a sticky end); and (ii) the second synaptic complex includes a second pair of oligomerized transposases, wherein one member of the pair is bound to a second nucleic acid including a TBS and a ligatable end (e.g., a sticky end); and (b) ligating the first synaptic complex to the second synaptic complex by ligating the ligatable ends (e.g., sticky ends) of the first nucleic acid and the second nucleic acid in a ligation reaction, thereby preparing a TSC. The first nucleic acid and the second nucleic acid can have the same nucleic acid sequence or can have different nucleic acid sequences. In some instances, (a) further includes providing a linking segment nucleic acid including a first ligatable end (e.g., a sticky end) and a second ligatable end (e.g., a sticky end). In some instances, the first ligatable end and/or the second ligatable end is a sticky end. The first sticky end may be compatible with the sticky end of the first nucleic acid, and the second sticky end may compatible with the sticky end of the second nucleic acid. In such instances, (b) may include ligating the linking segment nucleic acid to the first nucleic acid and the second nucleic acid. In some instances, the linking segment includes one or more additional elements selected from the group consisting of IST, a primer binding site, a cleavage site, or a chemical modification (e.g., biotinylation).

Another exemplary method for making a TSC can include one or more (e.g., 1, 2, 3, or all 4) of the following steps: (a) making a transposable nucleic acid that minimally includes a fully active double-stranded TBS at one terminus and an inactive single-stranded TBS at the other terminus; (b) adding transposase protein to the transposable nucleic acids to form dimers bound together through the ends with fully active double-stranded TBSs (the free ends on the dimers are inactive single-stranded TBSs); (c) converting the single-stranded TBSs on the dimer to double-stranded TBSs that are fully active for transposase binding by: (i) hybridizing a complementary nucleic acid to each single-stranded TBS; (ii) ligating a hybridized nucleic acid to form a double-stranded TBS; or (iii) extending a nucleic acid with DNA polymerase to form a double-stranded TBS; and (d) adding sufficient transposase protein to bind all of the dimers with fully active double-stranded TBSs to form a higher order TSC.

As described herein, TSCs in the current invention can be used to form physical bridges between distal locations on the same target DNA molecule, which can be exploited, for example, to determine linkage and phasing information. TSCs can be designed so that the DNA termini in any given TSC subunit will attach at the same target DNA sequence, but the nearest synaptic complex to which the first synaptic complex is tethered ligates DNA at a distal location usually in the same target DNA molecule. In nature, the distance between TBSs on a nucleic acid molecule needs to be large enough to permit successful transposition of a protein-encoding transposon (e.g., encoding proteins for antibiotic resistance and transposase) because it confers properties necessary for survival of the host. If two terminal TBSs present on a nucleic acid molecule are too close together, constraints on nucleic acid (e.g., DNA) bending will prevent the transposases bound to termini on the same molecule from forming a synaptic complex. However there is no such steric constraint on synaptic complex formation between terminal TBSs present on different nucleic molecules, which is a property that can be exploited to make TSCs.

If identical transposase binding sites are positioned sufficiently close to one another, the precise geometry and DNA bending associated with dimerization and synaptic complex formation is sterically favored between neighboring transposable nucleic acid molecules in a TSC as illustrated in FIG. 1. The length (e.g., in bp) between terminal TBSs on a nucleic acid molecule can be varied in order to promote oligomerization and synaptic complex between neighboring nucleic acid molecules in a TSC. A skilled artisan will appreciate that, in some cases, the length may vary between different types of transposases, but routine approaches can be used to determine whether a given length is suitable for use in making TSCs. For example, <64 bp of DNA separating two Tn5 TBSs on a plasmid DNA dramatically inhibited IS50 transposition activity in vivo in E. coli (Goryshin et al., Proc. Natl. Acad. Sci. USA 91:10834-10838, 1994). Similarly, in a preferred embodiment, we have discovered that distal transposition is promoted when TSCs are formed in vitro from transposase protein preparations and synthetic transposable nucleic acid molecules carrying two closely spaced TBSs.

Many transposases have been shown to distort nearby DNA conformation upon binding to the TBS. With respect to Tn5 transposase, for example, the bending angle on DNA is approximately 119⁰ and centers near the first and third nucleotide of the 19 bp transposase binding site (Jilk et al., J. Bacteriol. 178:1671-1679, 1996). To one of ordinary skill in the art, it would be understood that the relative three-dimensional (3-D) orientation of the reactive ends of a transposable nucleic acid can be modified by changing the distance between the transposase binding sites because the pitch and length of the DNA helix influences the orientation of the reactive ends in 3-D space. One would predict that as the distance between TBSs is reduced to less than 100 bp, the rigidity of double-stranded DNA will eventually prevent the interaction of the TBSs present on both ends of the same DNA molecule. This model is consistent with observations in vivo made by Goryshin (ibid) who described a striking periodic relationship between the DNA length separating the TBSs on plasmid DNA and the IS50 transposition frequency for lengths between 66 and 174 bp, with the transposition activity maxima corresponding to 10.5 bp intervals, which is identical to the helical repeat length of various linear DNAs in solution. This suggests that in the context of TSCs, the average distance linking distal transposition events in target DNA can be modulated by changing the relative 3-D orientation of the reactive ends on the face of the tethered synaptic complexes (e.g., by modifying the distance between transposase binding sites).

Methods by which the average distance between distal transposition events can be controlled can also broadly include methods known to increase the rigidity or diffusion of nucleic acids, such as by adding a molecule that increases the rigidity of the spacer region (linking segment) separating TBSs on transposable nucleic acid, including, but not limited to the following classes of molecules with known DNA binding properties: nucleic acid stains and nucleic acid intercalators (e.g., acridine dyes (e.g., acridine orange) and ethidium bromide), certain antibiotics, or DNA binding proteins; by modifying the nucleic acid content between TBSs on a transposable nucleic acid with biotin, and then adding streptavidin protein to bind the biotin-modified spacer region, thereby decreasing the flexibility of the spacer region separating transposase binding sites; by adding molecules known to bind, precipitate, and/or condense DNA into toroidal structures, such as histones or histone-like proteins, protamine, spermidine, hexamine cobalt chloride, polyethylene glycol, and the like; by immobilization of extended tethered synaptic complexes on a solid substrate; or by synthesizing a transposable nucleic acid on a solid or semi-solid surface.

When the use of longer nucleic acids for separating transposase binding sites is desired, a TSC could show unwanted transposase activity toward itself rather than toward target DNA. It also will be understood to one skilled in the art that there are means by which the TSC can be modified to make it resistant to unwanted transposase activity. Exemplary, non-limiting ways that a TSC can be rendered more resistant to transposase include the following: the TSC could contain a nucleotide analog resistant to transposase; the TSC could be designed having an overall G+C composition of less than 30%, which, in the case of Tn5 transposase, is known to be transposase-resistant; the TSC could be designed to be rich in sequences known to be a poor substrate for one or more transposases; the TSC could be made partly single stranded; the TSC could be coated with a DNA binding protein; if biotinylated, the nucleic acid between transposase binding sites could be coated with streptavidin or avidin protein; or the TSC could be immobilized to a solid substrate, or synthesized in situ to prevent unwanted transposition into TSC DNA.

Another method for making TSCs involves using nucleic acids having TBSs for transposases that do not oligomerize with each other on the same nucleic acid molecule to promote the formation of heterofunctional TSCs (see, for example, FIG. 2B). For example, one can make a nucleic acid having the TBS for Tn5 transposase at one end and the TBS for Mu transposase at the other end. Although this list is not comprehensive, many different combinations of transposase proteins and TBSs could be used to create heterofunctional TSCs, including known TBSs, such as those for Tn3, Tn5, Tn9, Tn10, gamma-delta and Mu transposases. Heterofunctional TSCs can be formed using mixtures of transposases, but they are joined exclusively through the termini of neighboring transposable nucleic acid molecules because by design, no single nucleic molecule carries TBSs for the same transposase. Heterofunctional TSCs can also be formed by linking synaptic complexes containing two different types of transposases (see, e.g., FIGS. 3, 4A, 4B, 4C, and 4D). The linkage can be non-covalent (e.g., via an affinity binding pair) or covalent (e.g., via a conjugation reaction, including ligation reactions). It will also be evident to one of ordinary skill in the art that active transposase mutants can be isolated or engineered and that these biologically active variants may exhibit different binding specificities than the parent or reference transposase from which they were derived (similar to those mutants described by Naumann et al., Proc. Natl. Acad. Sci. USA 97:8944-8949, 2000).

As one of ordinary skill in the art will appreciate, synthetic analogs of nucleic acids can substitute for naturally-occurring nucleic acids in many molecular biology procedures, including all the procedures and compositions described herein. Incorporation of modified bases and/or nuclease recognition sites can allow for optional separation of the TBSs later in any of the procedures. Any of the methods of making TSCs described herein may involve use of nucleic acids that include nucleic acid analogs, modified bases, and/or nuclease recognition sites.

TSCs can be used immediately after they are made, or stored for later use (e.g., for days, weeks, months, or years). The TSCs can be stored at any suitable temperature (e.g., about −80° C., about −20° C., about 0° C., about 15° C., about 20° C., about 25° C., about 37° C., or higher). The TSCs may be stored in any suitable storage buffer, which may exclude magnesium and manganese, and include one or more additional components, such as stabilizing agents, monovalent cations, cryoprotectants (e.g., glycerol or sucrose), anti-microbial agents, nuclease inhibitors, chelators, non-ionic surfactants, and the like. Storage buffers for nucleic acids and proteins (e.g., transposases) are known in the art.

TSCs can be prepared using transposable nucleic acid of different lengths for different levels of spatial resolution; or the ordering of the TSCs can be influenced by the order of addition of TSC subunits to transposase (or transposase to subunits). The length of TSCs can be adjusted, for example, by adding transposable nucleic acids each carrying a TBS at only one terminus. Terminating the TSCs in this manner also can serve to minimize or prevent undesired polymerization of distinct subpools of TSCs. TSCs of a particular length can be separated from lower weight nucleic acids that fail to form high molecular weight TSCs using a variety of separation methods known to those skilled in molecular biology, including but not limited to gel filtration, ultrafiltration, preparative gel electrophoresis, chromatography, density gradient ultracentrifugation, or by selectively precipitating or by binding polymers of the desired length to a solid substrate using polyethylene glycol or similar compounds.

Any desired number of subunits may be incorporated into a TSC oligomer or polymer. For example, an oligomer or polymer may include at least about 3, about 4, about 5, about 6, about 7, about 8, about 9, about 10, about 15, about 20, about 25, about 30, about 35, about 40, about 45, about 50, about 60, about 70, about 80, about 90, about 100, about 200, about 300, about 400, about 500, about 600, about 700, about 800, about 900, about 1000, or more subunits.

The ease with which transposase activity is reconstituted in vitro from a few components is why simpler transposases such as Tn5 transposase are often preferred over transposases requiring substantially longer DNA binding sites and/or several accessory proteins to reconstitute transposase activity. However, a skilled artisan will appreciate that the disclosure of the present invention allows a skilled artisan to use any suitable transposase, TBS, and, if relevant, accessory protein(s) to make TSCs falling within the scope of the invention.

### Methods of Using Tethered Synaptic Complexes

The compositions and methods of the invention are useful in a wide variety of applications, such as applications in which it is desirable to introduce nucleic acid sequences (for example, containing identifiable sequence tags and/or primer binding sites) into a target nucleic acid (e.g., DNA, such as genomic DNA), including, for example, preparation of libraries for nucleic acid sequencing. In general, the TSCs of the invention may be used in methods that can involve combining a target nucleic acid (e.g., DNA, such as genomic DNA) with one or more compositions of the invention under conditions suitable for transposition of transposable nucleic acid molecules at distal sites in the target nucleic acid. A primary mode by which the compositions and methods of the present invention differ from others known in the art is that after combining a target nucleic acid such as DNA with a TSC, each transposable nucleic acid molecule that tethers two synaptic complexes in the TSC is covalently attached at distal locations in the target DNA in two distinct molecular transposition events. In contrast, current practices typically attach two adapter molecules at the same location in the target DNA in a single molecular transposition event. An advantage of attaching one transposable nucleic acid molecule to two distal locations is that the probability of attachment can, under suitable conditions, be constrained by or related to the distance between the attachment sites in the target DNA. Establishing direct linkages between local and distal sites on the same DNA molecule reveals the organization of DNA on a scale that far exceeds the read length limitations of current DNA sequencing technologies.

The broad utility of the present invention extends to many areas of nucleic acid (e.g., DNA) sequencing. One example of the utility of the invention is in allowing for information regarding the phasing of mutations as having arisen either in cis or in trans with respect to a target DNA or reference sequence of interest. Now referring to FIG. 11, there is shown an example where the linking of identifiable sequence tags derived from treating a target DNA mixture with a plurality of TSCs can be used to phase mutations. In FIG. 11, two instances of TSCs with identifiable sequence tags are shown being used to treat two copies of a target DNA that have variants with respect to each other at two locations (i.e. one copy 802 has “A” and “B”, the other copy 801 has “a” and “b”). Because the transposition of the identifiable sequence tags for each pair of synaptic complexes in a TSC occurs in cis, the resulting pairs of sequenceable fragments 803 and 804 (derived, for example, by the process of FIG. 7) will contain the identifiable sequence tags that allow for proper phasing of the mutations observed on each fragment.

Any of the methods of the invention in which TSCs are brought into contact with target DNA can include a step of modifying the target DNA to bring normally distant sites into an orientation where TSCs can more readily covalently bridge one distal site in the target DNA and another. One clear challenge addressed by the present invention is overcoming the natural propensity of transposases to form a synaptic complex with the nearest available transposase binding site to ligate transposable DNA to opposing strands at precisely the same location in the target DNA molecule. The nearest available transposase binding site is normally present on the same DNA molecule. By generating TSCs in which the TBSs are present at regular intervals, the range of potential molecular interactions is reduced, thereby increasing the reliability with which certain behaviors can be predicted. If one simultaneously restrains the range of movement of the target DNA (e.g., by binding it to a substrate or scaffold or exposing it to an agent that causes DNA supercoiling, condensation, or precipitation), it is expected that the combined effects to be a more highly ordered system with properties that can be modified to suit the needs of the application. For example, if the target DNA was less than 10 kilobases in length, one could add target DNA to TSCs in a fully extended, native state, because the target DNA compaction would be unnecessary to detect linked, long-range transposition events over such a relatively short span. Regardless, any of the TSCs described herein can include a plurality of synaptic complexes that are about equidistant from one another, and these TSCs or any others can be used in methods that include a step of restraining the range of movement of the target DNA.

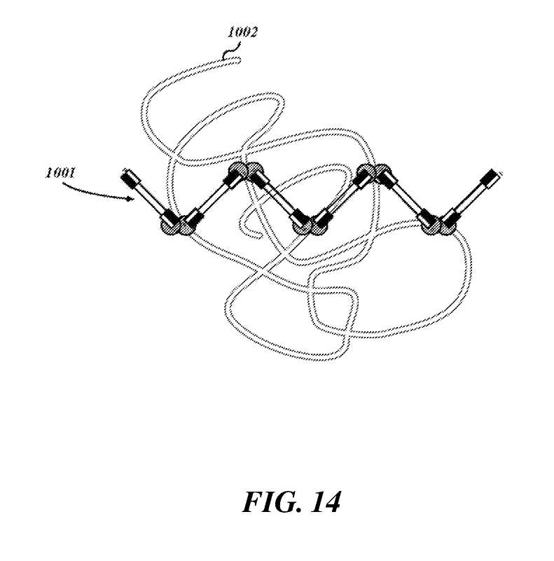

In some preferred embodiments, the action of a TSC on target DNA that has altered topological properties due to the presence of binding, precipitating or condensing agents, will have enhanced utility due to the fact that such agents may cause sites that are ordinarily more distal in a target DNA molecule to come within closer physical co-proximity. FIG. 13 shows, for example, an embodiment where a TSC (901) is used to treat DNA (903) that is bound or wrapped around a histone-like protein (902). An alternate embodiment, shown in FIG. 14, shows a TSC (1001) being used to treat DNA (1002) that is in a relatively condensed state.

The compositions of the invention (e.g., nucleic acids and TSCs) can be used in a number of transposition methods, for example, for use in preparing libraries for sequencing. Exemplary methods are described further below.

An example of a one-step transposition method may include one or more (e.g., 1, 2, 3, 4, or all 5) of the following steps: (a) adding a TSC to a target DNA; (b) adding DNA polymerase to fill in gaps in DNA; (c) enriching for library fragments carrying long distance linkage information (e.g., by amplifying by polymerase chain reaction (PCR) or any suitable method); (d) sequencing library fragments in parallel (e.g., using NGS); and (e) identifying linkages between library fragments conveyed by transposed nucleic acid sequences (e.g., identifiable sequence tags).

An example of a two-step transposition method may include one or more (e.g., 1, 2, 3, 4, 5, or all 6) of the following steps: (a) adding a TSC to a target DNA; (b) adding a conventional transposase reagent to add priming sites for amplification-based (e.g., PCR) enrichment of products of linked, but separate transposition events; (c) adding DNA polymerase to fill-in gaps in DNA; (d) enrich for library fragments carrying long distance linkage information; (e) sequencing library fragments in parallel (e.g., using NGS); and (f) identifying linkages between library fragments conveyed by transposed nucleic acid sequences (e.g., identifiable sequence tags).

An example of an alternate two-step transposition method that involves use of a first transposase and a second transposase may include one or more (e.g., 1, 2, 3, 4, 5, 6, or all 7) of the following steps: (a) adding a first transposase to a nucleic acid that includes a TBS at each terminus to form synaptic complexes (leaving out the second transposase); (b) adding synaptic complexes prepared in the previous step to target DNA to initiate a first transposition reaction and allow to proceed to completion, wherein the majority of the first transposase and synaptic complexes are consumed; (c) adding the second transposase to the products of the first transposition reaction to initiate a second transposition reaction; (d) adding DNA polymerase to fill-in gaps in DNA; (e) enriching for DNA fragments carrying long distance linkage information, for example, using amplification by PCR; (f) sequencing library fragments in parallel; and (g) identifying linkages between library fragments conveyed by transposed nucleic acid sequences (e.g., identifiable sequence tags).

An exemplary rationale for the alternate two-step transposition method described in the preceding paragraph is that the average distance between transposed nucleic acids (e.g., identifiable sequence tags) inserted into target DNA can be controlled by adjusting the concentration of the first synaptic complex reagent relative to the concentration of the target DNA (where higher relative concentration of the first synaptic complex reagent or lower concentration of target DNA will result in closer spacing of the inserted transposable nucleic acid molecules). The synaptic complex will insert at a single site in target DNA in the first step because the TBS at one end remains free until the second transposase is added. After completion of the first transposition reaction, the second transposase is added, and the free ends on the transposed nucleic acids form active synaptic complexes with the second transposase and a second transposition reaction proceeds, attaching the other end of the transposed nucleic acid in target DNA locations proximal to the insertions catalyzed by the first transposition step.

An example of a three-step transposition method that involves use of a first transposase and a second transposase may include one or more (e.g., 1, 2, 3, 4, 5, 6, 7, 8, or all 9) of the following steps: (a) adding the first transposase protein to bind two nucleic acid molecules together through TBSs to form synaptic complexes (the second transposase protein is temporarily withheld); (b) adding the synaptic complexes prepared in the previous step to target DNA to initiate a first transposition reaction and allowing the reaction to proceed to completion where the first transposase and synaptic complexes are consumed; (c) adding the second transposase to the products of the first transposition reaction to initiate a second transposition reaction; (d) optionally adding a nuclease to cleave the transposed nucleic acid at specific locations (e.g., a cleavage site); (e) adding a conventional transposase reagent (e.g., a tagmentation reagent such as Illumina NEXTERA™) to add priming sites for PCR enrichment of products of linked, but separate transposition events in a third transposition reaction; (f) adding DNA polymerase to fill-in gaps in DNA; (g) enriching for library fragments carrying long distance linkage information, for example, using amplification by PCR; (h) sequencing library fragments in parallel; and (i) identifying linkages between library fragments conveyed by transposed nucleic acid sequences (e.g., identifiable sequence tags).

The present invention is broadly useful for the purpose of determining the distance separating linked DNA molecules. A single nucleic acid molecule can be made (e.g., synthesized) carrying at least one fully-formed TBS for one transposase and a partially- or fully-formed TBS for the same or a different transposase, as described above. In this particular embodiment of a two-step transposition reaction, the transposable nucleic acid preparation is incubated with a first transposase protein to form a first mixture of synaptic complexes, and then added to a target DNA sample to initiate a first round of transposition events. Adding more synaptic complex to a fixed amount of target DNA will cause the average distance separating transposition events to be smaller. After the first transposition reaction, a DNA polymerase and deoxynucleotide triphosphates (dNTPs) are added, causing DNA extension to complete the formation of a second transposase binding site on the same adaptor. If a different transposase protein is to be used for the second transposition step (described below), then a nucleic acid with a fully formed transposase binding site for the second transposase can be used from the beginning of the procedure.

To prepare additional synaptic complexes from nucleic acids that are already inserted into target DNA, more transposase is added after completion of the first transposition reaction and after the transposase binding sites for the second transposition step are made active. The second transposition reaction is initiated by adding a second DNA sample to the second active synaptic complexes under conditions that are suitable for the activity of the second transposase. The second transposition reaction links the first DNA sample to a second DNA sample.

In another example of a two-step transposition method, the first and second DNA samples are target and reference samples, respectively. The target DNA sample can be synthetic or natural DNA from any source, whether from plant, animal, microbe, virus, the environment, or, of unknown provenance. The reference DNA sample can also be from a synthetic or natural source where all or some of the reference DNA sequence is known. The reference DNA can serve several purposes in molecular biology techniques; for example, as an easily accessible reservoir of highly diverse index sequences for DNA labeling and DNA sequencing; for identifying remotely linked and immediately adjacent DNA library fragments generated from the same target DNA molecule via covalent linkage to reference DNA of known DNA sequence and length; for quantifying the diversity of a population of DNA fragments; for appending DNA with uniquely indexed sequences priming sites for amplification; and for approximating the distance separating two or more transposition events on the same target molecule by using the known distance between insertion sites on the reference DNA as a “measuring stick.”

One of ordinary skill in the art appreciates that either target DNA or a reference DNA can serve as substrate for the first transposition. In another embodiment, the reference DNA sample is supplied as a ready-to-use formulation in a kit, where the reference DNA reagent has already undergone the first transposition and has already been complexed with the second transposase, so that a kit end-user could mix a target DNA sample with the reference DNA reagent provided in the kit to initiate the next transposition reaction. This form of reference DNA, complexed with fully functional transposase is known as “activated reference DNA.”

In another aspect, the reference DNA is designed and produced to suit the needs of a particular DNA sequencing application. When the target DNA sample is large and complex, as is the case for human genomic DNA, the reference DNA can be selected or designed to offer a very large number of unique insertion sites so that with sufficient sequencing depth adjacent library fragments can be confidently identified by transposition of a synaptic complex into a unique site on the reference DNA. For any given target DNA sample, the length of the unique reference DNA (in bp) offered should typically exceed the number of target molecules that one intends to sequence by two or more orders of magnitude.

Inserting mixed bases at certain points or interspersed at regular intervals in known reference DNA is a means by which one can generate a large diversity of reference DNA quickly and inexpensively for DNA sequencing. Although known DNA from a natural source could serve as a suitable DNA substrate for preparing reference DNA, synthetic reference DNA has clear advantages because the desirable properties for DNA sequencing can be altered at will.

In yet another embodiment, a reference DNA sample is immobilized to constrain its movement while reacting with target DNA sample. For example, biotinylated reference DNA can be immobilized on streptavidin paramagnetic beads through specific sites to orient the reference DNA for productive interaction with solution phase target DNA. Target DNA can also be immobilized or condensed before reacting with activated reference DNA.

In another aspect, a collection of reference DNA samples can be arrayed in a dense format on a solid substrate in some recognizable pattern. The pattern of immobilized reference DNA can be created by one of, or a combination of, the many methods widely known to practitioners of molecular biology and to manufacturers of laboratory products, and especially known to manufacturers of microarrays and DNA sequencing platforms, such as methods for depositing beads or small droplets onto a solid surface or into microwells; for applying DNA or beads carrying DNA onto a surface for immobilization by pipetting, spotting, spraying, acoustic dispensing, or piezoelectric dispensing; or for synthesis of DNA directly on a surface. Next, a solution of target DNA molecules is applied to the surface of immobilized reference DNA by pipetting, spotting, flooding, or by flowing a solution through a microfluidic path (or by any of the other methods mentioned previously). In this embodiment, the addresses of the reference DNA samples are either known before the target DNA is applied to the immobilized reference; determined through DNA sequencing before or after the target DNA is applied to the immobilized reference; or determined by some other method or combination of methods known to molecular biologists for interrogating the relative position of DNA content, such as by hybridization of labeled oligonucleotides to the reference DNA or target DNA, or by polymerase extension of oligonucleotides from nucleic acids bound to the surface. In some instances, the immobilized target DNA sample can substitute for the immobilized reference DNA in these examples, while in other instances solution phase reference DNA could be applied to immobilized target DNA. When a reference DNA molecule (for example, an E. coligenomic DNA) is prepared as a tethered synaptic complex preserving its natural order and inserted via transposition at multiple sites along a target DNA molecule, the reference DNA sequence can serve as an identifiable sequence tag at known positions in the reference with known distances between the identifiable sequence tags and thereby conveys useful information about the ordering of the target DNA sequence.

In another example, activated reference DNA can be mixed with target DNA under conditions where the transposition reaction does not proceed (e.g., by withholding magnesium ions). It has been demonstrated that active transposases complexed with DNA (e.g., TSCs) are stable, but reference DNA could also be stored in an inactive form to which a transposase is added at some later point before use.

The mixture of target DNA and activated DNA mixture can be co-condensed by the addition of agents (e.g., polyethylene glycol, spermine, protamine, manganese, hexamine cobalt chloride, and the like) known to form DNA toroids or to precipitate DNA. By co-spooling two or more molecules into toroids, or by co-precipitating the DNA mixture, the activated reference and target DNA would be brought into close proximity for transposition. The DNA toroids or precipitates can be collected by centrifugation, filtration, binding to solid surface, or by another method for immobilization and removal of excess condensing/precipitation agents.

In a preferred embodiment, the reference DNA is relatively free of undesirable repeat sequences, regions of extreme base composition (e.g., low or high GC bias), insertional hotspots for transposases, homopolymer sequences, or any other DNA sequence that could interfere with the reliable production of reference DNA or of DNA sequencing.

In any of the embodiments described herein, the identifiable sequence tags on the two strands of each transposed nucleic acid can be, for example, continuous or discontinuous complementary randomers, which, after the so-called index read step in DNA sequencing, can be used to detect linkages between distal sites in target DNA bridged by a single transposed nucleic acid (FIG. 8), wherein detection of a repeated sequence in target DNA immediately downstream of the insertion site in different library fragments provides evidence that a neighboring pair of subunits in a TSC were attached to opposite strands at that location in target DNA in the same transposition event. The positions of the index and duplicated sequences correspond to known locations within the transposed DNA and target sequences, and as such, these positions can be queried automatically. If there is reference sequence information available for the expected target DNA sequence, then sequence data extending well beyond the duplicated sequence can support higher confidence long virtual sequencing reads.

The TSCs of the invention have a number of unanticipated advantages. For example, TSCs exhibit a strong transposition proximity bias that is likely due to the rafting behavior of TSCs combined with the tendency of transposase protein to remain tightly bound to the transposed nucleic acid after transposition, which greatly increases the likelihood that transposable nucleic acid molecules from the same TSC will attach to the same DNA molecule multiple times. In this context, “rafting” refers to the effect whereby polymeric or multimeric chains of more than two TSCs will associate in coordinated fashion with a target DNA, upon occurrence of a first binding and/or transposition event by one of the synaptic complexes that is part of a given TSC polymer. Also, the reverse complement of an identifiable sequence tag linking distal transposition events can be copied during a fill in step with DNA polymerase. Further, TSCs can be assembled in separate subpools with unique identifiers (e.g., identifiable sequence tags), allowing easier identification of target DNA islands within DNA sequencing datasets based on the rafting behavior of distinct TSC subpools.

### Library Preparation and Sequencing Methods

The compositions (e.g., TSCs) and methods described herein can be used, for example, to prepare target nucleic acids (e.g., DNA) for sequencing, for example, for library preparation. The invention also provides methods of sequencing target nucleic acids (e.g., DNA). Any suitable sequencing technique described herein or known in the art can be used in the context of the invention. The methods to determine the nucleotide sequence of a target nucleic acid can be automated (e.g., in a fully automated device). The methods preferably employ NGS approaches. These methods and their applications are described in additional detail below.

Methods of preparing a target nucleic acid (e.g., DNA) for sequencing may include combining a TSC of the invention with a target nucleic acid under conditions and for a time sufficient for the TSC to carry out a transposition event. The method may further include fragmenting the target nucleic acid and optionally adding a polynucleotide to the resulting ends of the nucleic acid fragments. Typically the reaction will occur in buffered solution compatible with transposition, of which many are known in the art (e.g., N-Tris(hydroxymethyl)methyl-3-aminopropanesulfonic acid (TAPS)-based buffers, see Picelli et al., Genome Res. 24:2033, 2014). The buffered solution will typically include any necessary cofactors, such as a divalent metal cation (e.g., magnesium cations). A skilled artisan appreciates that the exact conditions and time of the reaction may vary depending, for example, on the TSC (e.g., the transposase(s) that are used), the target nucleic acid, and the sequencing approach used. These conditions can be readily determined based on the present disclosure and routine approaches known in the art.

Any suitable method for fragmenting nucleic acids may be used, for example, physical fragmentation (e.g., sonification, acoustic shearing, nebulization, needle shearing, and hydrodynamic shearing), enzymatic fragmentation (e.g., using a nuclease (e.g., an endonuclease, such as DNasel, a restriction endonuclease (e.g., EcoRI, BamHI, EcoRV, and Clal), RNAselll, a transposase (e.g., Tn5), and the like), chemical fragmentation (e.g., using heat and a divalent metal cation such as magnesium or zinc, which may be used for fragmentation of long RNA fragments). The fragmentation may be random or non-random. For example, restriction endonucleases typically cleave DNA at specific sequences, while other enzymes, such as DNAsel, typically fragment DNA with relatively low sequence specificity. Fragmentation can result in fragments having a desired length (e.g., an average length for a population of fragments), for example, of about 10 bp, about 50 bp, about 100 bp, about 200 bp, about 300 bp, about 400 bp, about 500 bp, about 600 bp, about 700 bp, about 800 bp, about 900 bp, about 1000 bp, about 2000 bp, about 3000 bp, about 4000 bp, about 5000 bp, or higher.

In most transposase-based fragmenting methods, target DNA is treated with a purified transposase enzyme (e.g., Tn5) complexed with short synthetic oligonucleotides (e.g., containing transposase binding sites and other sequences of interest such as primer binding sites and/or identifiable sequence tags) to promote molecular transposition events producing a plurality of DNA fragments, instead of integrating a transposon into a target DNA. The methods in which purified transposases are used as reagents in artificial transpositions to prepare libraries for NGS are sometimes referred to as “tagmentation.” Tagmentation reagents are commercially available (e.g., Illumina NEXTERA™) or can be produced using standard approaches (see, e.g., Picelli et al., Genome Res. 24:2033, 2014).

Any suitable method for adding a polynucleotide to the resulting ends of the nucleic acid fragments may be used. For example, the method may include enzymatically “polishing” the ends of DNA fragments (e.g., using a DNA polymerase such as DNA polymerase I Klenow fragment, T7 DNA polymerase, Bst DNA polymerase, Taq, Pfu, and the like) to permit subsequent ligation of different adapter sequences onto the polished DNAs (for example, using DNA ligase) that allow random fragments of the original source DNA to be subsequently amplified efficiently and without bias. In another example, tagmentation approaches may result in addition of an adapter or barcode onto the ends of each fragment.

Methods of sequencing a target nucleic acid may include one or more (e.g., 1, 2, 3, 4, or all 5) of the following steps: combining a TSC with a target nucleic acid under conditions and for a time sufficient for the TSC to carry out a transposition event; (b) fragmenting the target nucleic acid and adding a polynucleotide to the resulting ends of the nucleic acid fragments; (c) selecting DNA fragments comprising a nucleic acid sequence resulting from the transposition event; (d) amplifying the selected fragments; and (e) sequencing the amplified fragments. In particular embodiments, (b) may include random sharing and adapter ligation (also known as “shotgun adaptation”) or tagmentation. The selecting of (c) may include selecting nucleic acid fragments that include an identifiable sequence tag. Any suitable method may be used for amplifying selected fragments, including, for example, polymerase chain reaction (PCR), multiple displacement amplification (MDA), ligase chain reaction (LCR), loop mediated isothermal amplification (LAMP), rolling circle amplification (RCA), or strand displacement amplification (SDA). Other amplification methods are known in the art and may be used in the invention. The sequencing of (e) may include any suitable sequencing approach, preferably an NGS sequencing approach such as sequencing-by-synthesis (SBS), sequencing-by-ligation (SBL), and nanopore sequencing. Exemplary sequencing approaches are described in more detail below. Any of the methods may further include (f) analyzing the sequenced fragments to identify fragments of the target nucleic acid that can be linked due to the presence of a nucleic acid sequence resulting from the transposition event.

In some instances, SBS may be utilized in the context of the invention. SBS techniques generally involve the enzymatic extension of a nascent nucleic acid strand through the iterative addition of nucleotides against a template strand. SBS techniques can utilize nucleotide monomers that have a terminator moiety or those that lack any terminator moieties. Some exemplary types of SBS that do not utilize a terminator moiety include ion semiconducting sequencing and pyrosequencing (see, e.g., Margulies et al., Nature 437(7057):376-80, 2005; Rothberg et al., Nat. Biotechnol. 10(26):1117-24, 2005; Merriman et al., Electrophoresis 23(33):3397-417, 2012; and U.S. Pat. Nos. 7,323,305; 8,546,128; 8,574,835; 8,673,627; 8,748,102; and 8,765,380). In pyrosequencing approaches, a desired DNA sequence is able to be determined by light emitted upon incorporation of the next complementary nucleotide, relying on the detection of pyrophosphate release on nucleotide incorporation. In ion semiconducting sequencing approaches, detection is based on the release of hydrogen ions during the polymerization of DNA.

For SBS techniques that utilize nucleotide monomers having a terminator moiety, the terminator can be irreversible under the sequencing conditions used as in traditional Sanger sequencing, which utilizes dideoxynucleotides, or the terminator can be reversible, as is the case for sequencing methods developed by Solexa (now Illumina, Inc.) (see, e.g., U.S. Pat. Nos. 5,750,341; 6,255,475; and 6,355,431).

In such an embodiment, the DNA to be sequenced is modified to enable attachment to a flow cell via complementary sequences. As the DNA is amplified, fluorescently tagged nucleotides are added to the DNA strand, with one base added per amplification round as a result of a reversible terminator on every nucleotide, and light emission is detected by a camera.

SBS techniques that involve real-time monitoring of DNA polymerase activity can also be used.

For example, in SMRT™ sequencing, a zero-mode waveguide (ZMW) is utilized, wherein the ZMW is a structure that creates an observation volume small enough to observe a fluorescent signal emitted when a single nucleotide of DNA is incorporated into the nascent strand (see, e.g., Levene et al., Science 299(5607):682-6, 2003; Eid et al., Science 323(5910):133-8, 2009; Chin et al., Nat. Methods 6(10):563-9, 2013; and U.S. Pat. Nos. 7,181,122; 7,302,146; and 7,313,308). In such embodiments, the illumination can be restricted to a zeptoliter-scale volume around a surface-tethered polymerase such that incorporation of fluorescently labeled nucleotides can be observed with low background.

SBL techniques can also be used in the context of the invention. Examples of SBL include, without limitation, polony sequencing and sequencing by oligonucleotide ligation and detection (SOLiD™) (see, e.g., Mitra et al., Anal., Biochem. 320(1):55-65, 2003; Shendure et al., Science 309(5741):1728-32, 2005; Cloonan et al., Nat. Methods 5(7):613-9, 2008; and U.S. Pat. No. 9,243,290). SBL uses the DNA ligase enzyme to identify the nucleotide present at a given location in a DNA sequence, relying on DNA ligase's mismatch sensitivity instead of second strand synthesis. Detection of fluorescently-labeled probe oligonucleotides is typically performed with each cycle of ligation.

Nanopore sequencing can also be used. Nanopore sequencing is a real-time DNA sequencing technique in which target nucleic acids pass through a nanopore (see, e.g., Cockroft et al., J. Am. Chem. Soc. 3(130):818-20, 2008; Feng et al., Genomics Proteomics Bioinformatics 1(13):4-16, 2015; Fuller et al., Proc. Nat. Acad. Sci. USA 19(113):5233-8, 2016; U.S. Pat. No. 7,001,792; and U.S. Patent Application Publication Nos. 2011/0177493 and 2016/0076092). The nanopore can be a synthetic pore or biological membrane protein. As the target nucleic acid passes through the nanopore, each base-pair can be identified by measuring fluctuations in the electrical conductance of the pore.

The compositions (e.g., nucleic acids and TSCs) and methods described herein can be used in any sequencing application, particularly those in which incorporation of defined nucleic acid sequences (e.g., identifiable sequence tag(s)) into a target nucleic acid (e.g., DNA) is desired. The compositions and methods can be used to obtain fully phased, resolved sequence information and can overcome the length limitation imposed by most NGS instruments. Exemplary, non-limiting applications of the present invention include whole-genome sequencing, single-cell genome sequencing, exome sequencing, RNA sequencing (RNA-seq), genome-wide haplotype sequencing, epigenomics, and transcriptomics.

Additional applications of next-generation sequencing are also known in the art, and the compositions and methods of the invention may be used in any suitable application.

In some instances, the compositions (e.g., nucleic acids and TSCs) and methods described herein may be utilized in whole-genome or whole-exome sequencing, for example, for identifying disease-causing genetic variations, including indels, non-synonymous variants, or splice-site variants (see, e.g., Cirulli et al., Nat. Rev. Genet. 11(6):415-25, 2010). In some embodiments, the invention can be utilized in high-throughput RNA sequencing (RNA-seq), with specific applications including gene expression profiling and splice junction analysis (see, e.g., Li et al., Nat. Biotechnol. 32(9):915-25, 2014). In still other embodiments, the compositions (e.g., nucleic acids and TSCs) and methods may be utilized in genome-wide haplotype sequencing, with specific applications including mutation phase assessment (see, e.g., Snyder et al., Nat. Rev. Genet. 16(6):344-58, 2015). As an example, the compositions and methods of the invention can be used to obtain phase-resolved human leukocyte antigen (HLA) typing.

In other instances, the compositions (e.g., nucleic acids and TSCs) and methods described herein may be utilized in epigenomic applications, including chromatin immunoprecipitation followed by high-throughput sequencing (ChlP-seq), DNA methylation analysis through bisulfite sequencing, and chromatin footprinting (see, e.g., Zentner et al., Nat. Rev. Genet. 15(12):814-27, 2014; Park, Nat. Rev. Genet. 10(10):669-80, 2009; Brunner et al., Genome Res. 19(6):1044-56, 2009; and Buenrostro et al., Nat. Methods 10(12):1213-8, 2013). In other embodiments, the compositions (e.g., nucleic acids and TSCs) and methods described herein may be utilized in single-cell genome sequencing, with specific applications including de novo assembly of genomes, copy number variant detection, and single nucleotide variant detection (see, e.g., Gawad et al., Nat. Rev. Genet. 17(3):175-88, 2016).

### Target Nucleic Acids

Any target nucleic acid may be combined with a composition of the invention (e.g., a TSC), for example, for library preparation and sequencing. For example, the target nucleic acid may be DNA, RNA, peptide nucleic acid, morpholino nucleic acid, locked nucleic acid, glycerol nucleic acid, hybrids thereof, and mixtures thereof. The target nucleic acid can be of any suitable length, e.g., about 10 bp, about 20 bp, about 50 bp, about 100 bp, about 200 bp, about 500 bp, about 1000 bp, about 5000 bp, about 10,000 bp, about 20,000 bp, about 50,000 bp, about 100,000 bp, about 250,000 bp, about 500,000 bp, about 750,000 bp, about 1 million bp, about 5 million bp, about 10 million bp, about 15 million bp, about 20 million bp, or more. The target nucleic acid may include any sequence, and may include homopolymer sequences or repeat sequences. The repeat sequences can be of any of a number of lengths, e.g., about 2, about 5, about 6, about 7, about 8, about 9, about 10, about 12, about 15, about 20, about 25, about 30, about 35, about 40, about 45, about 50, about 100, about 250, about 500, about 1000 nucleotides, or more. Repeat sequences may be repeated contiguously or non-contiguously, for example, 2, 3, 4, 5, 6, 7, 8, 9, 10, 20, 30, or more times.

The target nucleic acid may be a single target nucleic acid, or there may be a plurality of target nucleic acid (e.g., tens, hundreds, thousands, millions, or more) target nucleic acids. Each member of the plurality of target nucleic acids may be the same, or each member may be different. The target nucleic acid can be synthetic or natural DNA from any source, whether from a plant, an animal (particularly a mammal such as a human), a microbe (e.g., from prokaryotes such as a bacterium (e.g., Escherichia coli, Staphylococcus aureus) or an archaeon, or from a eukaryote such as a fungus (e.g., budding yeast)), a virus, the environment, or, of unknown provenance. The target nucleic acid(s) may represent at least a portion of an organism's genome (e.g., at least about 1%, 5%, 10%, 20%, 25%, 30%, 40%, 50%, 75%, 80%, 90%, 95%, 99%, or 100% of the organism's genome). The target nucleic acid may be a chromosome. The target nucleic acid may include genomic DNA or cDNAs from a single cell. The target nucleic acid may include nucleic acids from a plurality of haplotypes.

### Kits

The invention provides kits that include one or more compositions of the invention (e.g., nucleic acids, molecular constructs, and TSCs). The kits may include one or more additional reagents that are useful, for example, for carrying out the methods of the invention. The kit may include one or more containers for holding the components of the kit (e.g., tubes (e.g., microcentrifuge tubes), plates (e.g., microtiter plates), trays, packaging materials, and the like. The kit may also include instructions (e.g., printed instructions for using the kit).

For example, a kit may include a TSC. Any of the TSCs described herein may be included in a kit. The TSC may include, for example, between two and ten thousand synaptic complexes. In some instances, each artificial nucleic acid in the TSC includes an identifiable sequence tag. Each identifiable sequence tag in the TSC may be identical, or the TSC may include a plurality of different identifiable sequence tags. In some embodiments, each identifiable sequence tag in the TSC is different.

A kit may include any of the nucleic acids described herein. For example, an exemplary kit may include an artificial nucleic acid that includes a first end comprising a first TBS, a second end comprising a second TBS, and a linking segment disposed between the first TBS and the second TBS. In some embodiments, upon binding of a first transposase to the first TBS and a second transposase to the second TBS, the first transposase does not oligomerize with the second transposase. The kit may also include a purified transposase that binds to the first TBS or the second TBS. The nucleic acid and purified transposase(s) can be present in the same container or in different containers. In some instances, the linking segment includes an identifiable sequence tag. The kit may include artificial nucleic acids each having the same identifiable sequence tag. In other examples, the kit may include a plurality of artificial nucleic acids, in which each member has a different identifiable sequence tag. A kit may also include any of the preceding artificial nucleic acids, a first transposase, and a second transposase, wherein the first transposase binds to the first TBS and the second transposase binds to the second TBS. A kit may include one or more components of an affinity binding pair (e.g., biotin-biotin binding protein (e.g., avidin, streptavidin, or NeutrAvidin™)). A kit may include a reagent used in a conjugation reaction, including catalysts (e.g., copper), ligands, and other reagents.

Any of the preceding kits may include one or more additional reagents. For example, the one or more additional reagents may include a cofactor, a buffered solution, and/or a reference nucleic acid. The cofactor may be a divalent metal cation (e.g., a magnesium cation). Any of the kits may also include a reagent for nucleic acid sequencing, which may include, for example, oligonucleotide primer(s), a substrate, an enzyme (e.g., a DNA polymerase), a mixture of nucleotides, and/or a reference nucleic acid.

### EXAMPLES

The following are examples of methods and compositions of the invention. It is understood that various other embodiments may be practiced, given the general description provided above.

### Example 1. Preparation of Heterofunctional Tethered Synaptic Complexes (TSCs)

Heterofunctional TSCs containing synaptic complexes made of two different types of transposase proteins tethered by transposable nucleic acid molecules were prepared as follows. To generate donor DNA that was a suitable substrate for formation of TSCs, E. coli DNA was used as a template in a series of PCR reactions using modified oligonucleotide primers designed to produce terminal TBS regions at opposing ends of the PCR products. The PCR amplification was performed using a series of outward nested PCR reactions as follows.

In the first PCR (PCR-1), specific regions of E. coli DNA were amplified with tailed primers (100 bp.mu, 100 bp.tn5) and Q5® DNA polymerase (New England BioLabs). The following primer sequences were used:

PCR-1 primer sequences:
100 bp.mu:
(SEQ ID NO: 31)
5′-GTT TCA CGA TAA ATG CGA AAA CAA AAC CAT CGC CGA
GAT TTG CC-3′
100 bpn5:
(SEQ ID NO: 32)
5′-CTG TCT CTT ATA CAC ATC TAA TTT GCT GCC TTC CTG
AAT GC-3′.

In the second PCR (PCR-2), the product of PCR-1 was amplified using the primers Mu145 and Tn5.MEDS.UNIV using Q5® DNA polymerase (New England BioLabs). The following primer sequences were used:

PCR-2 primer sequences:
MuL45:
(SEQ ID NO: 33)
5′-CGG CGC ACG AAA AAC GCG AAA GCG TTT CAC GAT AAA
TGC GAA AAC-3′
TN5.MEDS.UNIV:
(SEQ ID NO: 34)
5′-/PHOS/-CTG TCT CTT ATA CAC ATC T-3′.

In the third PCR (PCR-3), the product of PCR-2 was amplified using the primers NuMu and TN5.MEDS.UNIV using Q5® DNA polymerase (New England BioLabs). The following primer sequences were used:

PCR-3 primer sequences:
NuMu-
(SEQ ID NO: 35)
5′-GAA TGC AGA TCT GAA GCG CAC GAA AAA CGC GA-3′
TN5.MEDS.UNIV-
(SEQ ID NO: 34)
5′-/PHOS/-CTG TCT CTT ATA CAC ATC T-3′.

The 180 bp PCR amplicon resulting from PCR-3 is referred to as the “donor DNA” and has the following sequence:

(SEQ ID NO: 36)
5′-GAA TGC AGA TCT GAA GCG GCG CAC GAA AAA CGC GAA
AGC GTT TCA CGA TAA ATG CGA AAA CAA AAC CAT CGC
CGA GAT TTG CCC GTA GAT TTC AGT GCC GGT TAA CTC
CTC GAG CAA TTC CGC GCG TTC TTT GGG TTT GGC ATT
CAG GAA GGC AGC AAA TTA GAT GTG TAT AAG AGA CAG-
3′.

The donor DNA was purified with MAGwise™ paramagnetic beads (seqWell Inc.) following the manufacturer's operating instructions. The purified donor DNA was digested with BglII restriction enzyme (New England BioLabs) to form a suitable pre-cleaved end for MuA transposase activity. The BglII-digested 173 bp donor DNA has a MuA transposase binding site at one end, and a Tn5 transposase binding site at the opposite end, with the following sequence:

(SEQ ID: 37)
5′-GAT CTG AAG CGG CGC ACG AAA AAC GCG AAA GCG TTT
CAC GAT AAA TGC GAA AAC AAA ACC ATC GCC GAG ATT
TGC CCG TAG ATT TCA GTG CCG GTT AAC TCC TCG AGC
AAT TCC GCG CGT TCT TTG GGT TTG GCA TTC AGG AAG
GCA GCA AAT TAG ATG TGT ATA AGA GAC AG-3′.

The BglII-digested donor DNA was purified with MAGwise™ magnetic beads (seqWell Inc.), and quantified using PicoGreen® dsDNA assay (Thermo).

The purified, BglII-digested, donor DNA was incubated with MuA transposase to form MuA transposomes at molar binding ratios (MBR) (MuA MBR=MuA transposase:donor DNA) ranging from 0.5:1 to 3:1. To determine the MuA MBR with maximal MuA transposase activity, the level of MuA transposition was assessed by treating a pUC19 plasmid DNA standard with the MuA transposomes formed at different MBRs. The MuA MBR reactions were performed in 1× transposase reaction buffer (25 mM Tris-HCl pH 8.0 at 20° C.; 10 mM MgCl₂; 110 mM NaCl; 0.05% TRITON® X-100; 10% glycerol). The level of DNA fragmentation resulting from MuA transposition was observed by agarose gel electrophoresis.

Separately, Tn5 transposomes were formed by incubating Tn5 transposase with purified, BglII-digested, donor DNA at molar binding ratios (Tn5 MBR=Tn5 transposase:donor DNA) ranging from approximately 10:1 to 20:1. To determine the Tn5 MBR that corresponded to the maximal Tn5 transposase activity, the Tn5 transposition activity was assessed by treating a pUC19 plasmid DNA standard with the Tn5 transposomes formed at different MBRs. The Tn5 MBR reactions were performed in 1× transposase reaction buffer (see above). The level of DNA fragmentation resulting from Tn5 transposition was observed by agarose gel electrophoresis.

To produce heterofunctional TSCs containing both MuA and Tn5 transposases, MuA and Tn5 MBRs corresponding to the maximum respective transposase activities from the experiments described above were selected and used to prepare heterofunctional TSCs as follows. MuA transposase was incubated with BglII-digested donor DNA for 2 hours at 30° C. using the maximal MuA MBR to form MuA transposomes, as described above. Tn5 transposase was then added to the MuA transposomes using the maximal Tn5 MBR as observed above, and incubated for an additional hour at room temperature to form heterofunctional TSCs. The resulting MuA-Tn5 TSC reagent was stored on ice, or at 4° C. for longer periods.

### Example 2: Use of TSCs to Prepare Libraries for DNA Sequencing

Libraries were prepared for DNA sequencing using the MuA-Tn5 TSC reagent described in Example 1. In these experiments, the plasmid pUC19 served as target DNA. 200 ng of pUC19 plasmid DNA was treated with an amount of MuA-Tn5 TSC reagent sufficient to cleave 50% of the pUC19 DNA approximately one time, as observed by conversion of closed circular DNA to linear DNA as assessed by agarose gel electrophoresis. The MuA-Tn5 TSC was mixed with pUC19 DNA and incubated at 30° C. for 1 hour, followed by incubation at 55° C. for 15 min. This reaction was performed in 1× transposase reaction buffer (see Example 1). A volume of 0.2% sodium dodecyl sulfate (SDS) equal to₁/10^{th} the volume of the initial reaction was then added and the reaction was heat-inactivated at 72° C. for 10 min. The heat-inactivated reaction was diluted to 200 μl with ultrapure water, and DNA was recovered from the reaction by purification with MAGwise™ paramagnetic beads.

Insertion of donor DNA by the MuA and Tn5 transposases of the TSC introduces random gaps and nicks in the target pUC19 DNA. To repair the transposase-modified pUC19 DNA, the purified DNA was treated with PreCR® Repair Mix (New England BioLabs). The PreCR® repair reaction was incubated at 37° C. for 30 minutes and then diluted by addition of 200 μl of ultrapure water. The repaired pUC19 DNA containing donor DNA from the MuA-Tn5 TSC reagent inserted at random sites was purified with MAGwise™ paramagnetic beads.

The purified and repaired pUC19 DNA containing random donor DNA insertions was subsequently tagmented with T1T and T2T reaction mixtures (plexWell™ Library Preparation Kit (seqWell Inc.)) according to the manufacturer's instructions. The reaction mixtures contained the following tagging reagent sequences:

T1T tagging reagent sequence:
TAG1.A01:
(SEQ ID NO: 38)
5′-CAA GCA GAA GAC GGC ATA CGA GAT AAC ACC TAG TCT
CGT GGG CTC GGA GAT GTG TAT AAG AGA CAG-3′
T2T tagging reagent sequence:
TAG2.D02:
(SEQ ID NO: 39)
5′-AAT GAT ACG GCG ACC ACC GAG ATC TAC ACC TGC TTC
GTC GTC GGC AGC GTC AGA TGT GTA TAA GAG ACA G-3′.

The tagmentation reaction was incubated at 55° C. for 15 min, followed by incubation at 72° C. for 5 min. After completion of the tagging reaction, 5 μl of 0.2% SDS was added to the reaction mixture. The reaction was heat-inactivated by incubating first at 50° C. for 15 min and then at 68° C. for 5 min. The heat-inactivated reaction was diluted to 200 μl with 10 mM Tris-HCl, pH8 and purified by adding 140 μl of room temperature MAGwise™ magnetic beads (seqWell Inc.) according to the manufacturer's instructions. The tagmented and purified DNA library was eluted in 30 μl of 10 mM Tris-HCl, pH 8.

The DNA library fragments were prepared for PCR amplification by adding 25 μl of KAPA HiFi™ HotStart ReadyMix (2×) (Kapa Biosystems) to 25 μl of the purified tagged DNA, and incubated at 72° C. for 10 min. 4.8 μl of a primer mixture containing 5 μM each of oligo P5 and oligo P7 were added to the reaction. The P5 and P7 library amplification primer sequences are as follows:

P5:
(SEQ ID NO: 40)
5′-AAT GAT ACG GCG ACC ACC GAG-3′
P7:
(SEQ ID NO: 41)
5′-CAA GCA GAA GAC GGC ATA CGA G-3′.

The library fragments were amplified by PCR using the following thermocycle parameters:

1. Denature the DNA at 95° C. for 3 min

2. Incubate at 98° C. for 20 sec

3. Incubate at 62° C. for 30 sec

4. Incubate at 72° C. for 30 sec

5. Return to step 2, 21 times

6. Incubate at 72° C. for 3 min

7. Hold at 10° C.

The PCR amplified library fragments were diluted to 200 μl with ultrapure water and purified by addition of 130 μl of room temperature MAGwise™ paramagnetic beads (seqWell Inc.). The DNA was purified according to the manufacturer's instructions and eluted in 30 μl of 10 mM Tris-HCl, pH 8. The purified library was then quantified using qPCR (KAPA Library Quantification Kit, Kapa Biosystems), and prepared for DNA sequencing on the Illumina MiSeq™ system according to the manufacturer's instructions.

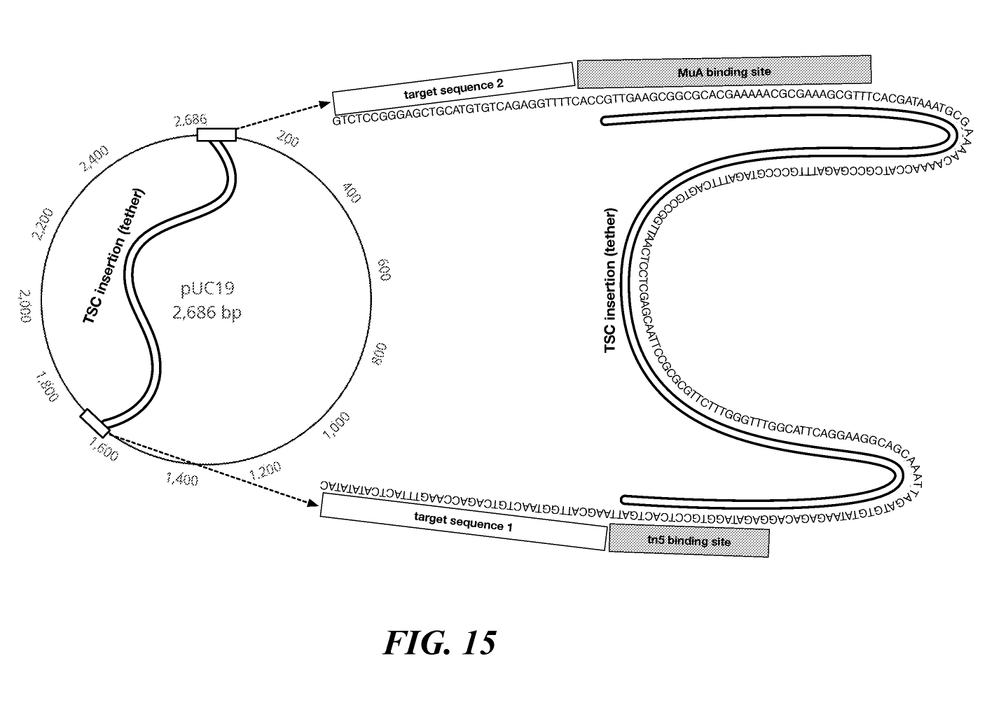

The results of the sequencing confirmed that the MuA-Tn5 TSC reagent had transposed donor DNA into the pUC19 plasmid, thereby linking two distal sites. FIG. 15 shows one representative sequencing read that contained the sequence of the transposed donor DNA, including the MuA and Tn5 binding sites, as well as sequences from the pUC19 plasmid that were separated by approximately 1-2 kb (note that the pUC19 plasmid is circular). Accordingly, these results show that TSCs can be used to insert known nucleic acid cargo molecules into distal sites in a target nucleic acid molecule, for example, during library preparation for next-generation sequencing.

### Example 3: Use of Affinity Binding Pairs in Preparation of TSCs

Two or more nucleic acids containing TBSs can be linked using affinity binding pairs to generate TSCs for use in the methods of the invention. As one example, a nucleic acid having the sequence of the 5′ half of SEQ ID NO:37 and a nucleic acid having the sequence of the 3′ half of SEQ ID NO:37 can each be biotinylated using standard approaches in the art (e.g., using a Pierce Biotin 3′ End DNA Labeling Kit or by biotinylation during synthesis). In other examples, two nucleic acids each having a Tn5 TBS can be used. The biotinylated nucleic acids are mixed with streptavidin in a suitable buffer (e.g., 1× transposase reaction buffer (25 mM Tris-HCl pH 8.0 at 20° C.; 10 mM MgCl2; 110 mM NaCl; 0.05% TRITON® X-100; 10% glycerol)). Transposases (e.g., Mu and/or Tn5) are added to the resulting constructs, for example, as described in Example 1, to form TSCs. The TSCs can be used in any of the methods described herein (see, e.g., Example 2).

### Example 4: Use of Conjugation Reactions in Preparation of TSCs

Two or more nucleic acids containing TBSs can be linked using one or more conjugation reactions to generate TSCs for use in the methods of the invention. As one example, a nucleic acid having the sequence of the 5′ half of SEQ ID NO:37 and a nucleic acid having the sequence of the 3′ half of SEQ ID NO:37 can be conjugated using an azide-alkyne Huisgen cycloaddition (e.g., a copper(I)-catalyzed azide-alkyne cycloaddition (CuAAC) or a strain-promoted azide-alkyne cycloaddition (SPAAC)). In other examples, two nucleic acids each having a Tn5 TBS can be used. The nucleic acids can be conjugated directly to each other, or can be conjugated to a linking segment (e.g., DNA, protein, or a non-nucleotide chemical moiety (e.g., a polymer (e.g., a polyether such as PEG))). Transposases (e.g., Mu and/or Tn5) are added to the resulting constructs, for example, as described in Example 1, to form TSCs. The TSCs can be used in any of the methods described herein (see, e.g., Example 2).

What is claimed is:

1. A tethered synaptic complex (TSC) comprising:
a first artificial nucleic acid comprising a first end comprising a first transposase binding site (TBS), a second end comprising a second TBS, and a linking segment disposed between the first TBS and the second TBS;
a second artificial nucleic acid comprising a first end comprising a first TBS;
a third artificial nucleic acid comprising a first end comprising a first TBS;
a first synaptic complex comprising a first pair of oligomerized transposases, the first pair comprising a first transposase and a second transposase, wherein the first transposase is bound to the first TBS of the first artificial nucleic acid, and the second transposase is bound to the first TBS of the second artificial nucleic acid; and
a second synaptic complex comprising a second pair of oligomerized transposases, the second pair comprising a third transposase and a fourth transposase, wherein the third transposase is bound to the second TBS of the first artificial nucleic acid, and the fourth transposase is bound to the first TBS of the third artificial nucleic acid.

2. The TSC of claim 1, wherein the linking segment comprises a nucleic acid.

3. The TSC of claim 2, wherein the nucleic acid is at least partially single-stranded.

4. The TSC of claim 2, wherein the nucleic acid is double-stranded.

5. The TSC of any one of claims 1-4, wherein the linking segment comprises terminal nucleotides that form phosphodiester bonds with the first TBS and the second TBS.

6. The TSC of any one of claims 1-4, wherein the linking segment comprises an affinity binding pair or a covalent bond resulting from a conjugation reaction that does not form a phosphodiester bond.

7. The TSC of claim 6, wherein the affinity binding pair comprises biotin-streptavidin, biotin-avidin, ligand-receptor, antigen-antibody or antigen binding fragment, hapten-anti-hapten, or immunoglobulin (Ig) binding protein-Ig.

8. The TSC of claim 7, wherein the affinity binding pair comprises biotin-streptavidin or biotin-avidin.

9. The TSC of claim 8, wherein the streptavidin or avidin binds only one or two biotin molecules.

10. The TSC of any one of claims 6-9, wherein the affinity binding pair comprises a first affinity component that binds to two second affinity components, where one second affinity component is linked to the first end of the first artificial nucleic acid, and the other second affinity component is linked to the second end of the first artificial nucleic acid, and wherein the two second affinity reagents do not interfere with binding of transposases to the first and second TBSs of the first artificial nucleic acid.

11. The TSC of any one of claims 6-9, wherein the affinity binding pair comprises a first affinity component that binds a second affinity component, where the first affinity component is linked to the first end of the first artificial nucleic acid, and the second affinity component is linked to the second end of the first artificial nucleic acid.

12. The TSC of claim 6, wherein the conjugation reaction is selected from the group consisting of a cycloaddition, amide or thioamide bond formation, a pericyclic reaction, a Diels-Alder reaction, sulfonamide bond formation, alcohol or phenol alkylation, a condensation reaction, disulfide bond formation, and a nucleophilic substitution.

13. The TSC of claim 12, wherein the cycloaddition is an azide-alkyne Huisgen cycloaddition.

14. The TSC of claim 13, wherein the azide-alkyne Huisgen cycloaddition is a copper(I)-catalyzed azide-alkyne cycloaddition (CuAAC) or a strain-promoted azide-alkyne cycloaddition (SPAAC).

15. The TSC of any one of claims 1-14, wherein the linking segment further comprises one or more additional elements selected from the group consisting of an identifiable sequence tag (IST), a primer binding site, a cleavage site, and a chemical modification.

16. The TSC of claim 15, wherein the one or more additional elements is an IST.

17. The TSC of claim 15 or 16, wherein the IST is a random IST, a semi-random IST, or a non-random IST.

18. The TSC of claim 15, wherein the cleavage site is a restriction endonuclease recognition site or a nickase site.

19. The TSC of any one of claims 2-18, wherein the first artificial nucleic acid is about 50 to about 500 base pairs (bp) long.

20. The TSC of claim 19, wherein the first artificial nucleic acid is about 100 to about 250 bp long.

21. The TSC of claim 20, wherein the first artificial nucleic acid is about 150 to about 200 bp long.

22. The TSC of claim 21, wherein the first artificial nucleic acid is about 175 bp long.

23. The TSC of any one of claims 1-22, wherein the transposases of the first pair are a different type than the transposases of the second pair.

24. The TSC of any one of claims 1-23, wherein the transposases of the first pair and/or the second pair are Tn3, Tn5, Tn9, Tn10, gamma-delta, Mu, piggyBac, Minos, Tc1, or Sleeping Beauty transposases or biologically active variants thereof.

25. The TSC of claim 24, wherein the transposases of the first pair and/or the second pair are Tn3, Tn5, Tn9, Tn10, gamma-delta, or Mu transposases or biologically active variants thereof.

26. The TSC of claim 24 or 25, wherein the transposases of the first pair and/or the second pair are Tn5 or Mu transposases or biologically active variants thereof.

27. The TSC of any one of claims 24-26, wherein the transposases of the first pair are Tn5 transposases, and the transposases of the second pair are Mu transposases.

28. The TSC of any one of claims 1-27, wherein at least one transposase of the first pair and/or the second pair is operably linked to a targeting moiety.

29. The TSC of claim 28, wherein the targeting moiety is a polypeptide comprising a DNA-binding domain (DBD) or an RNA-guided endonuclease.

30. The TSC of claim 29, wherein the DBD is a zinc finger motif or a transcription activator-like (TAL) effector.

31. The TSC of claim 29, wherein the RNA-guided endonuclease is Cas9, Cpf1, C2c2, or a biologically active variant thereof.

32. The TSC of claim 31, wherein the biologically active variant is a nuclease-deficient variant.

33. The TSC of any one of claims 1-32, wherein the second artificial nucleic acid or the third artificial nucleic acid further comprises a second end, wherein the second end is a ligatable end.

34. The TSC of claim 33, wherein the ligatable end is a sticky end.

35. The TSC of any one of claims 1-32, wherein the second artificial nucleic acid or the third artificial nucleic acid further comprises a component of a second affinity binding pair or a conjugating moiety.

36. The TSC of claim 35, wherein the conjugating moiety is selected from the group consisting of a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a sulfhydryl, a thiol, and a thioisocyanate group.

37. The TSC of any one of claims 1-32, wherein the second artificial nucleic acid further comprises a second end comprising a second TBS, and a linking segment disposed between the first TBS and the second TBS of the second artificial nucleic acid.

38. The TSC of claim 37, wherein the linking segment of the second artificial nucleic acid comprises a second affinity binding pair or a covalent bond resulting from a conjugation reaction.

39. The TSC of any one of claims 1-32, 37, or 38, wherein the third artificial nucleic acid further comprises a second end comprising a second TBS, and a linking segment disposed between the first TBS and the second TBS of the third artificial nucleic acid.

40. The TSC of claim 39, wherein the linking segment of the third artificial nucleic acid comprises a third affinity binding pair or a covalent bond resulting from a conjugation reaction.

41. The TSC of any one of claims 1-32, or 37-40, further comprising:
one or more additional synaptic complexes, each additional synaptic complex comprising a pair of oligomerized transposases, and/or
one or more additional artificial nucleic acids, each additional artificial nucleic acid comprising a TBS at each end and an intervening linking segment,
wherein each of the first synaptic complex, the second synaptic complex, and the one or more additional synaptic complexes is tethered to at least one other synaptic complex of the TSC by binding to TBSs at either end of the same artificial nucleic acid.

42. The TSC of claim 41, wherein the TSC comprises between one and ten thousand additional synaptic complexes.

43. The TSC of claim 41 or 42, wherein each artificial nucleic acid of the TSC comprises an IST.

44. The TSC of claim 43, wherein each IST is identical.

45. The TSC of claim 43, wherein each IST is not identical.

46. The TSC of any one of claims 1-45, wherein the linking segment of the first artificial nucleic acid is soluble in an aqueous solution.

47. The TSC of any one of claims 1-46, wherein the linking segment of the first artificial nucleic acid has a mass of less than 1 femtogram.

48. An artificial nucleic acid comprising a first end comprising a first transposase binding site (TBS), a second end comprising a second TBS, and a linking segment disposed between the first TBS and the second TBS, wherein upon binding of a first transposase to the first TBS and a second transposase to the second TBS, the first transposase does not oligomerize with the second transposase.

49. The nucleic acid of claim 48, wherein the linking segment comprises a nucleic acid.

50. The nucleic acid of claim 49, wherein the nucleic acid is at least partially single-stranded.

51. The nucleic acid of claim 49, wherein the nucleic acid is double-stranded.

52. The nucleic acid of any one of claims 48-51, wherein the linking segment comprises terminal nucleotides that form phosphodiester bonds with the first TBS and the second TBS.

53. The nucleic acid of any one of claims 48-51, wherein the linking segment comprises an affinity binding pair or a covalent bond resulting from a conjugation reaction.

54. The nucleic acid of claim 53, wherein the affinity binding pair comprises biotin-streptavidin, biotin-avidin, ligand-receptor, antigen-antibody or antigen binding fragment, hapten-anti-hapten, or Ig binding protein-lg.

55. The nucleic acid of claim 54, wherein the affinity binding pair comprises biotin-streptavidin or biotin-avidin.

56. The nucleic acid of claim 55, wherein the streptavidin or avidin binds only one or two biotin molecules.

57. The nucleic acid of any one of claims 53-56, wherein the affinity binding pair comprises a first affinity component that binds to two second affinity components, where one second affinity component is linked to the first end of the first artificial nucleic acid, and the other second affinity component is linked to the second end of the first artificial nucleic acid.

58. The nucleic acid of any one of claims 53-56, wherein the affinity binding pair comprises a first affinity component that binds a second affinity component, where the first affinity component is linked to the first end of the first artificial nucleic acid, and the second affinity component is linked to the second end of the first artificial nucleic acid.

59. The nucleic acid of claim 53, wherein the conjugation reaction is selected from the group consisting of a cycloaddition, amide or thioamide bond formation, a pericyclic reaction, a Diels-Alder reaction, sulfonamide bond formation, alcohol or phenol alkylation, a condensation reaction, disulfide bond formation, and a nucleophilic substitution.

60. The nucleic acid of claim 59, wherein the cycloaddition is an azide-alkyne Huisgen cycloaddition.

61. The nucleic acid of claim 60, wherein the azide-alkyne Huisgen cycloaddition is a copper(I)-catalyzed azide-alkyne cycloaddition (CuAAC) or a strain-promoted azide-alkyne cycloaddition (SPAAC).

62. The nucleic acid of any one of claims 48-61, wherein the linking segment further comprises one or more additional elements selected from the group consisting of an IST, a primer binding site, a cleavage site, and a chemical modification.

63. The nucleic acid of claim 62, wherein the one or more additional elements comprises an IST.

64. The nucleic acid of claim 62 or 63, wherein the IST is a random IST, a semi-random IST, or a non-random IST.

65. The nucleic acid of claim 62, wherein the cleavage site is a restriction endonuclease recognition site or a nickase site.

66. The nucleic acid of any one of claims 49-65, wherein the nucleic acid is about 50 bp to about 500 bp in length.

67. The nucleic acid of claim 66, wherein the artificial nucleic acid is about 100 to about 250 bp long.

68. The nucleic acid of claim 67, wherein the artificial nucleic acid is about 150 to about 200 bp long.

69. The nucleic acid of claim 68, wherein the artificial nucleic acid is about 175 bp long.

70. The nucleic acid of any one of claims 48-69, wherein the linking segment prevents the first transposase and the second transposase from oligomerizing when bound to the first TBS and the second TBS.

71. The nucleic acid of any one of claims 48-70, wherein the first transposase and the second transposase do not oligomerize with each other.

72. The nucleic acid of any one of claims 48-71, wherein the first TBS or the second TBS is double-stranded.

73. The nucleic acid of any one of claims 48-72, wherein more than one transposase binds to the first TBS or the second TBS.

74. A molecular construct comprising a first transposase, a second transposase, and the artificial nucleic acid of any one of claims 48-73, wherein the first transposase is bound to the first TBS and the second transposase is bound to the second TBS.

75. The molecular construct of claim 74, wherein the linking segment prevents the first transposase and the second transposase from oligomerizing with each other.

76. The molecular construct of claim 75, wherein the first transposase and the second transposase do not oligomerize with each other.

77. The molecular construct of any one of claims 74-76, wherein the first transposase or the second transposase is a Tn3, Tn5, Tn9, Tn10, gamma-delta, Mu, piggyBac, Minos, Tc1, or Sleeping Beauty transposase or a biologically active variant thereof.

78. The molecular construct of claim 77, wherein the first transposase or the second transposase is a Tn3, Tn5, Tn9, Tn10, gamma-delta, or Mu transposase or a biologically active variant thereof.

79. The molecular construct of claim 77 or 78, wherein the first transposase or the second transposase is a Tn5 transposase, a Mu transposase, or a biologically active variant thereof.

80. The molecular construct of any one of claims 74-79, wherein the first transposase is a Tn5 transposase and the second transposase is a Mu transposase.

81. The molecular construct of any one of claims 74-80, wherein more than one transposase binds to the first TBS or the second TBS.

82. The molecular construct of any one of claims 74-81, wherein the first transposase or the second transposase is operably linked to a targeting moiety.

83. The molecular construct of claim 82, wherein the targeting moiety is a polypeptide comprising a DNA-binding domain (DBD) or an RNA-guided endonuclease.

84. The molecular construct of claim 83, wherein the DBD is a zinc finger motif or a transcription activator-like (TAL) effector.

85. The molecular construct of claim 83, wherein the RNA-guided endonuclease is Cas9, Cpf1, C2c2, or a biologically active variant thereof.

86. The molecular construct of claim 85, wherein the biologically active variant is a nuclease-deficient variant.

87. A TSC comprising at least three of the molecular constructs of any one of claims 74-86, wherein the constructs are concatenated by oligomerization of a transposase in each construct with a transposase in another construct, and wherein at least two synaptic complexes are present in the TSC.

88. The TSC of claim 87, wherein the TSC comprises between two and ten thousand synaptic complexes.

89. The TSC of claim 87 or 88, wherein each artificial nucleic acid in the TSC comprises an IST.

90. The TSC of claim 89, wherein each IST is identical.

91. The TSC of claim 89, wherein each IST is not identical.

92. A method of preparing a TSC, the method comprising:
(a) providing at least a first synaptic complex and a second synaptic complex, wherein
(i) the first synaptic complex comprises a first pair of oligomerized transposases, wherein one member of the pair is bound to a first nucleic acid comprising a TBS and a sticky end; and
(ii) the second synaptic complex comprises a second pair of oligomerized transposases, wherein one member of the pair is bound to a second nucleic acid comprising a TBS and a sticky end; and
(b) ligating the first synaptic complex to the second synaptic complex by ligating the sticky ends of the first nucleic acid and the second nucleic acid in a ligation reaction,
thereby preparing a TSC.

93. The method of claim 92, wherein the first nucleic acid and the second nucleic acid have the same nucleic acid sequence.

94. The method of claim 92, wherein the first nucleic acid and the second nucleic acid have different nucleic acid sequences.

95. The method of any one of claims 92-94, wherein (a) further comprises providing a linking segment nucleic acid comprising a first sticky end and a second sticky end, wherein the first sticky end is compatible with the sticky end of the first nucleic acid and the second sticky end is compatible with the sticky end of the second nucleic acid.

96. The method of claim 95, wherein (b) further comprises ligating the linking segment nucleic acid to the first nucleic acid and the second nucleic acid.

97. The method of claim 95 or 96, wherein the linking segment comprises one or more additional elements selected from the group consisting of IST, a primer binding site, a cleavage site, or a chemical modification.

98. The method of claim 97, wherein the chemical modification is a biotinylation.

99. A TSC prepared by the method of any one of claims 92-98.

100. A synaptic complex comprising:
(a) a first transposase;
(b) a second transposase;
(c) a first artificial nucleic acid comprising a first end comprising a TBS and being linked to a first conjugating moiety other than a 3′ hydroxyl; and
(d) a second artificial nucleic acid comprising a first end comprising a TBS and being linked to a second conjugating moiety other than a 3′ hydroxyl,
wherein the first transposase is bound to the TBS of the first artificial nucleic acid, the second transposase is bound to the TBS of the second artificial nucleic acid, and the first transposase and the second transposase are oligomerized.

101. The synaptic complex of claim 100, wherein the first and second conjugating moieties are the same.

102. The synaptic complex of claim 100, wherein the first and second conjugating moieties are the different.

103. The synaptic complex of claim 100, wherein the first and second conjugating moieties are independently selected from the group consisting of a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a sulfhydryl, a thiol, and a thioisocyanate group.

104. The synaptic complex of any one of claims 100-103, wherein the first or second artificial nucleic acid further includes an IST.

105. A method of preparing a TSC, the method comprising:
(a) providing at least a first synaptic complex and a second synaptic complex, wherein
(i) the first synaptic complex comprises a first pair of oligomerized transposases, wherein one member of the pair is bound to a first nucleic acid comprising a TBS and being linked to a first component of an affinity binding pair or a first conjugating moiety; and
(ii) the second synaptic complex comprises a second pair of oligomerized transposases, wherein one member of the pair is bound to a second nucleic acid comprising a TBS and being linked to a second component of the affinity binding pair or a second conjugating moiety; and
(b) linking the first synaptic complex to the second synaptic complex, wherein linking of the first conjugating moiety to the second conjugating moiety does not result in a phosphodiester bond,
thereby preparing a TSC.

106. The method of claim 105, wherein the linking of (b) comprises combining the first synaptic complex and the second synaptic complex under conditions suitable for binding of the first component and the second component of the affinity binding pair.

107. The method of claim 105, wherein the linking of (b) comprises conjugating the first conjugating moiety to the second conjugating moiety in a conjugation reaction that does not produce a phosphodiester bond.

108. A TSC prepared by the method of any one of claims 105-107.

109. A method of preparing a target nucleic acid for sequencing, the method comprising:
(a) combining the TSC of any one of claims 1-47, 87-91, 99, or 108 with a target nucleic acid under conditions and for a time sufficient for the TSC to carry out a transposition event.

110. The method of claim 109, further comprising:
(b) fragmenting the target nucleic acid and adding a polynucleotide to the resulting ends of the nucleic acid fragments.

111. A method of sequencing a target nucleic acid, the method comprising:
(a) combining the TSC of any one of claims 1-47, 87-91, 99, or 108 with a target nucleic acid under conditions and for a time sufficient for the TSC to carry out a transposition event;
(b) fragmenting the target nucleic acid and adding a polynucleotide to the resulting ends of the nucleic acid fragments;
(c) selecting DNA fragments comprising a nucleic acid sequence resulting from the transposition event;
(d) amplifying the selected fragments; and
(e) sequencing the amplified fragments.

112. The method of claim 110 or 111, wherein (b) comprises random shearing and adapter ligation or tagmentation.

113. The method of claim 111 or 112, wherein the selecting of (c) comprises selecting nucleic acid fragments comprising an IST.

114. The method of any one of claims 111-113, wherein the amplifying of (d) comprises polymerase chain reaction (PCR), multiple displacement amplification (MDA), ligase chain reaction (LCR), loop mediated isothermal amplification (LAMP), rolling circle amplification (RCA), or strand displacement amplification (SDA).

115. The method of any one of claims 111-114, wherein the sequencing of (e) comprises sequencing by synthesis, sequencing by ligation, or nanopore sequencing.

116. The method of claim 115, wherein the sequencing by synthesis comprises Illumina™ dye sequencing, single-molecule real-time (SMRT™) sequencing, or pyrosequencing.

117. The method of claim 115, wherein the sequencing by ligation comprises polony-based sequencing or SOLiD™ sequencing.

118. The method of any one of claims 111-117, further comprising:
(f) analyzing the sequenced fragments to identify fragments of the target nucleic acid that can be linked due to the presence of a nucleic acid sequence resulting from the transposition event.

119. The method of any one of claims 109-118, wherein the target nucleic acid comprises genomic DNA or cDNAs from a single cell.

120. The method of any one of claims 109-119, wherein the target nucleic acid comprises nucleic acids from a plurality of haplotypes.

121. The method of any one of claims 111-120, wherein the sequence of the amplified fragments is used to perform de novo sequence assembly.

122. A kit comprising the TSC of any one of claims 1-47, 87-91, 99, or 108.

123. A kit comprising the nucleic acid of any one of claims 48-73 and a purified transposase that binds to the first TBS or the second TBS.

124. A kit comprising the molecular construct of any one of claims 74-86.

125. The kit of any one of claims 122-124, further comprising one or more additional reagents selected from the group consisting of a cofactor, a buffered solution, and a reference nucleic acid.

126. The kit of claim 125, wherein the cofactor is a divalent metal cation.

127. The kit of claim 126, wherein the divalent metal cation is a magnesium cation.

128. The kit of any one of claims 122-127, further comprising a reagent for nucleic acid sequencing.

129. The kit of claim 128, wherein the reagent is selected from the group consisting of an oligonucleotide primer, a substrate, an enzyme, and a mixture of nucleotides.


SEQUENCE LISTING (42 sequences)

SEQ ID NO: 1 - DNA, 19 bp, Artificial Sequence
  Synthetic Construct
ctgactcttatacacaagt

SEQ ID NO: 2 - DNA, 19 bp, Artificial Sequence
  Synthetic Construct
ctgtctcttgatcagatct

SEQ ID NO: 3 - DNA, 19 bp, Artificial Sequence
  Synthetic Construct
ctgtctcttatacacatct

SEQ ID NO: 4 - DNA, 19 bp, Artificial Sequence
  Synthetic Construct
ctgtctcttatacagatct

SEQ ID NO: 5 - DNA, 26 bp, Artificial Sequence
  Synthetic Construct
tgtattgattcactgaagtacgaaaa

SEQ ID NO: 6 - DNA, 23 bp, Artificial Sequence
  Synthetic Construct
ccttaatcaatgaaacgcgaaag

SEQ ID NO: 7 - DNA, 23 bp, Artificial Sequence
  Synthetic Construct
ttgtttcattgaaaatacgaaaa

SEQ ID NO: 8 - DNA, 27 bp, Artificial Sequence
  Synthetic Construct
tgaagcggcgcacgaaaaatgcgaaaa

SEQ ID NO: 9 - DNA, 23 bp, Artificial Sequence
  Synthetic Construct
gcgtttcacgataaatgcgaaaa

SEQ ID NO: 10 - DNA, 23 bp, Artificial Sequence
  Synthetic Construct
ccgtttcatttgaagcgcgaaaa

SEQ ID NO: 11 - DNA, 22 bp, Artificial Sequence
  Synthetic Construct
  misc_feature (9)..(10): n is a, c, g, or t
ygtttcaynnraaryrcgaaar

SEQ ID NO: 12 - DNA, 23 bp, Artificial Sequence
  Synthetic Construct
ctgakrratcccctmatrattty

SEQ ID NO: 13 - DNA, 22 bp, Artificial Sequence
  Synthetic Construct
tgayaataaagttgattatact

SEQ ID NO: 14 - DNA, 38 bp, Artificial Sequence
  Synthetic Construct
ggggtctgacgctcagtggaacgaaaactcacgttaag

SEQ ID NO: 15 - DNA, 38 bp, Artificial Sequence
  Synthetic Construct
cttaacgtgagttttcgttccactgagcgtcagacccc

SEQ ID NO: 16 - DNA, 38 bp, Artificial Sequence
  Synthetic Construct
ggggtttgagggccaatggaacgaaaacgtacgttaag

SEQ ID NO: 17 - DNA, 38 bp, Artificial Sequence
  Synthetic Construct
ataaacgtacgttttcgttccattggccctcaaacccc

SEQ ID NO: 18-22 - skipped (5 entries)

SEQ ID NO: 23 - DNA, 255 bp, Artificial Sequence
  Synthetic Construct
cgcttaacttaatacgagccccaaccactattaattcgaacagcatgttttttttgcagtgcgcaatgtttaacacactatattatcaatactactaaagataacacataccaatgcatttcgtctcaaagagaattttattctcttcacgacgaaaaaaaaagttttgctctatttccaacaacaacaaaaatatgagtaatttattcaaacggtttgcttaagagataagaaaaaagtgaccactattaattc

SEQ ID NO: 24 - DNA, 255 bp, Artificial Sequence
  Synthetic Construct
atagtaaatcacattacgccgcgttcgaattaatagtggtcacttttttcttatctcttaagcaaaccgtttgaataaattactcatatttttgttgttgttggaaatagagcaaaactttttttttcgtcgtgaagagaataaaattctctttgagacgaaatgcattggtatgtgttatctttagtagtattgataatatagtgtgttaaacattgcgcactgcaaaaaaaacatgctgttcgaattaatagt

SEQ ID NO: 25 - protein, 476 aa, Artificial Sequence
  Synthetic Construct
MITSALHRAADWAKSVFSSAALGDPRRTARLVNVAAQLAKYSGKSITISSEGSEAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTLPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 26 - protein, 663 aa, Artificial Sequence
  Synthetic Construct
MELWVSPKECANLPGLPKTSAGVIYVAKKQGWQNRTRAGVKGGKAIEYNANSLPVEAKAALLLRQGEIETSLGYFEIARPTLEAHDYDREALWSKWDNASDSQRRLAEKWLPAVQAADEMLNQGISTKTAFATVAGHYQVSASTLRDKYYQVQKFAKPDWAAALVDGRGASRRNVHKSEFDEDAWQFLIADYLRPEKPAFRKCYERLELAAREHGWSIPSRATAFRRIQQLDEAMVVACREGEHALMHLIPAQQRTVEHLDAMQWINGDGYLHNVFVRWFNGDVIRPKTWFWQDVKTRKILGWRCDVSENIDSIRLSFMDVVTRYGIPEDFHITIDNTRGAANKWLTGGAPNRYRFKVKEDDPKGLFLLMGAKMHWTSVVAGKGWGQAKPVERAFGVGGLEEYVDKHPALAGAYTGPNPQAKPDNYGDRAVDAELFLKTLAEGVAMFNARTGRETEMCGGKLSFDDVFEREYARTIVRKPTEEQKRMLLLPAEAVNVSRKGEFTLKVGGSLKGAKNVYYNMALMNAGVKKVVVRFDPQQLHSTVYCYTLDGRFICEAECLAPVAFNDAAAGREYRRRQKQLKSATKAAIKAQKQMDALEVAELLPQIAEPAAPESRIVGIFRPSGNTERVKNQERDDEYETERDEYLNHSLDILEQNRRKKAI

SEQ ID NO: 27 - protein, 402 aa, Artificial Sequence
  Synthetic Construct
MCELDILHDSLYQFCPELHLKRLNSLTLACHALLDCKTLTLTELGRNLPTKARTKHNIKRIDRLLGNRHLHKERLAVYRWHASFICSGNTMPIVLVDWSDIREQKRLMVLRASVALHGRSVTLYEKAFPLSEQCSKKAHDQFLADLASILPSNTTPLIVSDAGFKVPWYKSVEKLGWYWLSRVRGKVQYADLGAENWKPISNLHDMSSSHSKTLGYKRLTKSNPISCQILLYKSRSKGRKNQRSTRTHCHHPSPKIYSASAKEPWVLATNLPVEIRTPKQLVNIYSKRMQIEETFRDLKSPAYGLGLRHSRTSSSERFDIMLLIALMLQLTCWLAGVHAQKQGWDKHFQANTVRNRNVLSTVRLGMEVLRHSGYTITREDLLVAATLLAQNLFTHGYALGKL

SEQ ID NO: 28 - protein, 273 aa, Artificial Sequence
  Synthetic Construct
MAKANSSFSEVQIARRIKEGRGQGHGKDYIPWLTVQEVPSSGRSHRIYSHKTGRVHHLLSDLELAVFLSLEWESSVLDIREQFPLLPSDTRQIAIDSGIKHPVIRGVDQVMSTDFLVDCKDGPFEQFAIQVKPAAALQDERTLEKLELERRYWQQKQIPWFIFTDKEINPVVKENIEWLYSVKTEEVSAELLAQLSPLAHILQEKGDENIINVCKQVDIAYDLELGKTLSEIRALTANGFIKFNIYKSFRANKCADLCISQVVNMEELRYVAN

SEQ ID NO: 29 - protein, 702 aa, Artificial Sequence
  Synthetic Construct
MWQINEVVLFDNDPYRILAIEDGQVVWMQISADKGVPQARAELLLMQYLDEGRLVRTDDPYVHLDLEEPSVDSVSFQKREEDYRKILPIINSKDRFDPKVRSELVEHVVQEHKVTKATVYKLLRRYWQRGQTPNALIPDYKNSGAPGERRSATGTAKIGRAREYGKGEGTKVTPEIERLFRLTIEKHLLNQKGTKTTVAYRRFVDLFAQYFPRIPQEDYPTLRQFRYFYDREYPKAQRLKSRVKAGVYKKDVRPLSSTATSQALGPGSRYEIDATIADIYLVDHHDRQKIIGRPTLYIVIDVFSRMITGFYIGFENPSYVVAMQAFVNACSDKTAICAQHDIEISSSDWPCVGLPDVLLADRGELMSHQVEALVSSFNVRVESAPPRRGDAKGIVESTFRTLQAEFKSFAPGIVEGSRIKSHGETDYRLDASLSVFEFTQIILRTILFRNNHLVMDKYDRDADFPTDLPSIPVQLWQWGMQHRTGSLRAVEQEQLRVALLPRRKVSISSFGVNLWGLYYSGSEILREGWLQRSTDIARPQHLEAAYDPVLVDTIYLFPQVGSRVFWRCNLTERSRQFKGLSFWEVWDIQAQEKHNKANAKQDELTKRRELEAFIQQTIQKANKLTPSTTEPKSTRIKQIKTNKKEAVTSERKKRAEHLKPSSSGDEAKVIPFNAVEADDQEDYSLPTYVPELFQDPPEKDES

SEQ ID NO: 30 - protein, 339 aa, Artificial Sequence
  Synthetic Construct
MGKSKEISQDLRKKIVDLHKSGSSLGAISKRLKPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWCGFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 31 - DNA, 44 bp, Artificial Sequence
  Synthetic Construct
gtttcacgataaatgcgaaaacaaaaccatcgccgagatttgcc

SEQ ID NO: 32 - DNA, 41 bp, Artificial Sequence
  Synthetic Construct
ctgtctcttatacacatctaatttgctgccttcctgaatgc

SEQ ID NO: 33 - DNA, 45 bp, Artificial Sequence
  Synthetic Construct
cggcgcacgaaaaacgcgaaagcgtttcacgataaatgcgaaaac

SEQ ID NO: 34 - DNA, 19 bp, Artificial Sequence
  Synthetic Construct
ctgtctcttatacacatct

SEQ ID NO: 35 - DNA, 32 bp, Artificial Sequence
  Synthetic Construct
gaatgcagatctgaagcgcacgaaaaacgcga

SEQ ID NO: 36 - DNA, 180 bp, Artificial Sequence
  Synthetic Construct
gaatgcagatctgaagcggcgcacgaaaaacgcgaaagcgtttcacgataaatgcgaaaacaaaaccatcgccgagatttgcccgtagatttcagtgccggttaactcctcgagcaattccgcgcgttctttgggtttggcattcaggaaggcagcaaattagatgtgtataagagacag

SEQ ID NO: 37 - DNA, 173 bp, Artificial Sequence
  Synthetic Construct
gatctgaagcggcgcacgaaaaacgcgaaagcgtttcacgataaatgcgaaaacaaaaccatcgccgagatttgcccgtagatttcagtgccggttaactcctcgagcaattccgcgcgttctttgggtttggcattcaggaaggcagcaaattagatgtgtataagagacag

SEQ ID NO: 38 - DNA, 66 bp, Artificial Sequence
  Synthetic Construct
caagcagaagacggcatacgagataacacctagtctcgtgggctcggagatgtgtataagagacag

SEQ ID NO: 39 - DNA, 70 bp, Artificial Sequence
  Synthetic Construct
aatgatacggcgaccaccgagatctacacctgcttcgtcgtcggcagcgtcagatgtgtataagagacag

SEQ ID NO: 40 - DNA, 21 bp, Artificial Sequence
  Synthetic Construct
aatgatacggcgaccaccgag

SEQ ID NO: 41 - DNA, 22 bp, Artificial Sequence
  Synthetic Construct
caagcagaagacggcatacgag

SEQ ID NO: 42 - DNA, 267 bp, Artificial Sequence
  Synthetic Construct
gtctccgggagctgcatgtgtcagaggttttcaccgttgaagcggcgcacgaaaaacgcgaaagcgtttcacgataaatgcgaaaacaaaaccatcgccgagatttgcccgtagatttcagtgccggttaactcctcgagcaattccgcgcgttctttgggtttggcattcaggaaggcagcaaattagatgtgtataagagacaggagataggtgcctcactgattaagcattggtaactgtcagaccaagtttactcatatatac